(19) United States
(12) Patent Application Publication
(10) Pub. No.: US 2005/0119305 A1
(43) Pub. Date: Jun. 2, 2005

(54) Il-6 production inhibitors

(76) Inventors: Masao Naka, Mishima-gun (JP); Kanji Takahashi, Mishima-gun (JP)

Correspondence Address: SUGHRUE MION, PLLC, 2100 PENNSYLVANIA AVENUE, N.W., SUITE 800, WASHINGTON, DC, 20037, US

(21) Appl. No.: 10/472,160

(22) PCT Filed: Mar. 20, 2002
(86) PCT No.: PCT/JP02/02681

(30) Foreign Application Priority Data
Mar. 21, 2001 (JP) 2001-081302

(51) Int. Cl. A61K 31/445

(52) U.S. Cl. 514/317

(57) ABSTRACT

An IL-6 production inhibitor which comprises a hydroxamic acid derivative of formula (I) 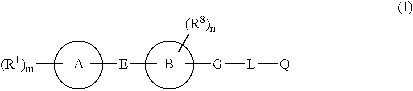 (wherein all the symbols have the same meanings as defined in the specification), an equivalent thereof, a non-toxic salt thereof or a prodrug thereof as an active ingredient. Because of having an IL-6 production inhibitory activity, the compound of formula (I) may be useful for the prevention and/or treatment of various inflammatory diseases, sepsis, multiple myeloma, plasma cell leukemia, osteoporosis, cachexia, psoriasis, nephritis, renal cell carcinoma, Kaposi's sarcoma, rheumatoid arthritis, hypergammaglobulinemia (gammophathy), Castleman's disease, intra-atrial myxoma, diabetes, autoimmune disease, hepatitis, colitis, graft-versus-host disease, infectious diseases, endometriosis and solid cancer.

### TECHNICAL FIELD

The present invention relates to
1) an IL-6 (Interleukin-6) production inhibitor which comprises a hydroxamic acid derivative of formula (I) 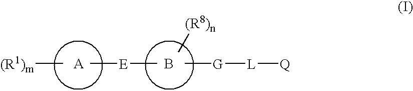
   (wherein all the symbols have the same meanings as defined hereinafter), an equivalent thereof, a non-toxic salt thereof or a prodrug thereof as an active ingredient,
2) a novel hydroxamic acid derivative or an equivalent thereof, and
3) a process for the preparation thereof.

### BACKGROUND ART

Cytokine is a multifunctional factor which plays an important role in the host defense system of living body and it relates to various life phenomena. However, there are many diseases which may be caused by overproduction thereof or overresponse thereto.

IL-6 is a cytekine produced from various cells, e.g. T cells, B cells, macrophages, kidney mesangial cells, fibroblasts, etc., and its various physiological effects are known e.g. induction of B cells differentiation to antibody-producing cells, activation of T cells, increase of platelets, production of acute phase protein from liver cells, etc. But an abnormal production of IL-6 has been observed in various inflammations, autoimmune diseases and neoplastic diseases and it is suggested that IL-6 plays a certain role in the causes of such pathophysiological situations. In the experiment using an animal model in which IL-6 was forcibly expressed, various types of diseases could be observed and such results strongly suggest the existence of relationship between the abnormal production of IL-6 and the cause of certain diseases (see Biochem. J., 265, 621 (1990); Immunol. Today, 11, 443 (1990); J. Autoimmun., 5 Suppl A, 123 (1992); Clin. Immunol. Immunopathol., 62, S60 (1992)).

Therefore inhibition of IL-6 production is expected to improve various kinds of diseases such as inflammatory diseases as a representative. The present invention targets these cytokines and provides medicines through inhibiting the production thereof.

Clinical application of the compound of the present invention involves those diseases which may be caused and be changed to worse by abnormal production of IL-6 or by overresponse thereto. An IL-6 production inhibitor may be used for the prevention and/or treatment of various inflammatory diseases, sepsis, multiple myeloma, plasma cell leukemia, osteoporosis, cachexia, psoriasis, nephritis, renal cell carinoma, Kaposi's sarcoma, rheumatoid arthritis, hypergammaglobulinemia (gammophathy), Castleman's disease, intra-atrial myxoma, diabetes, autoimmune disease, hepatitis, colitis, graft-versus-host disease, infectious diseases, endometriosis and solid cancer (e.g. brain tumor, head and neck tumor, thyroid cancer, esophageal cancer, gastric cancer, colorectal cancer (colon cancer and rectal cancer), liver cancer, gallbladder cancer, bileduct cancer, pancreatic cancer, breast cancer, cervical cancer, endometrial carcinoma, ovarian cancer, carcinoma of the prostate, orchioncus, bladder carinoma, renal pelvic tumor, ureteral tumor, adrenal tumor, neuroma, glioma, osteoncus, rhabdomyosarcoma, osteosarcoma, soft tissue tumors, oxyphilic granuloma, malignant melanoma, cutaneous carcinoma, glioblastoma, Wilms tumor, etc.) (see J. Immunol., 145, 4185 (1990); J. Exp. Med., 172, 1505 (1990); J. Clin. Invest., 87, 739 (1991); J. Clin. Invest., 89, 1681 (1992); EMBO J., 13, 1189 (1994); Hematol. Oncol. Clin. North Am., 11, 159 (1997); Cytokines Cell Mol. Ther., 4(3), 161 (1998); Folia Med. (Plovdiv), 41(1), 78 (1999); JPEN J. Parenteral Enteral Nutr., 23(5), S20 (1999); J. Infect. Dis., 180(1), 10 (1999); Am. J. Obstet. Gynecol., 176(3), 593 (1997)).

For example, in the specification of Japanese Patent Application Kokai S59-46244, it is disclosed that a hydroxamic acid derivative of formula (X)
A^{X}-B^{X}-(CH₂)_{nX}—CONHOH—(X)
wherein A^{X} is R^{X}X^{X}_{mX} (wherein R^{X} is phenyl, pyrrolyl, thienyl, imidazolyl or thiazolyl; X^{X} is halogen, lower alkyl, lower alkoxy or nitro; m^{X} is 0, 1 or 2 and m^{X} groups of X^{X} are the same or different optionally); B^{X} is —CHOH—, —CH—, —O— or —CO—; n^{X} is an integer of 2 to 10) is useful as an anti-protozoan agent.

And in the specification of U.S. Pat. No. 4,769,461, it is disclosed that a hydroxamic acid derivative of formula (Y) 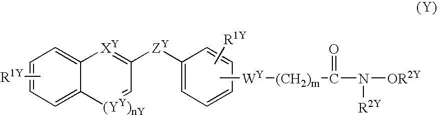
(wherein W^{Y} is bond, —O—, —S—, —NR^{2Y}—, —CH(OH)— or —NR^{2Y}—CO—; X^{Y} is N or CR^{2Y}; when n^{Y}=0, Y^{Y} is O, S, NR^{2Y} or C(R^{2Y})ₓ; when n^{Y}=1, Y^{Y} is N or CR^{2Y}; Z^{Y} is —CH₂O—, —CH₂S_{—, —CH}₂NR^{2Y}—, —O—, —S—, —NR^{2Y}—, —CO—, —CONR^{2Y}—, —CHR^{2Y}CHR^{2Y}—, —C(R^{2Y})═C(R^{2Y})— or —C≡C—; R^{1Y} is hydrogen, lower alkyl, trifluoromethyl, nitro, hydroxy, lower alkoxy, mercapto, lower alkylthio or halogen; R^{2Y} is hydrogen or lower alkyl; n^{Y} is 0 or 1; m^{Y} is 1 to 6; wherein W^{Y} is bond, then m^{Y} is 0 to 5) is useful as an anti-inflammatory or an anti-allergy agent by inhibition of cylooxygenase and lipoxygenase.

And in the specification of WO97/18188, it is disclosed that the compound of formula (Z) 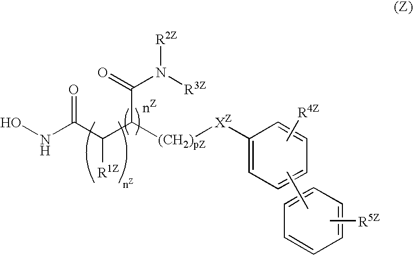
(wherein m^{Z} and n^{Z} are each independently 0 or 1; p^{Z} is 0 to 6; proviso, m^{Z}, n^{Z} and p^{Z} are not zero at same time, R^{1Z} is hydrogen, C1-6 alkyl, C2-6 alkenyl, hydroxy, etc.; R^{2Z} and R^{3Z} are each independently hydrogen, C1-6 alkyl, phenyl, pyridyl, etc; X^{Z} is bond, —O—, —NH—, —S—, etc.; R^{4Z} and R^{5Z} are each independently hydrogen, C1-6 alkyl, halogen, cyano, C1-6 cyanoalkyl, C1-6 haloalkyl, hydroxy or C1-6 alkoxy) is useful as an inhibitor of matrix metalloproteinase and TNF alpha secretion.

And in the specification of U.S. Pat. No. 4,731,382, it is disclosed that the compound of formula (W) 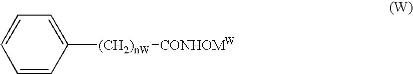
(wherein n is 6 to 11, M is hydrogen or cation) is useful as an inhibitor of 5-lipoxygenase.

### DISCLOSURE OF THE INVENTION

The present inventors have investigated to find a new compound possessing an inhibitory activity of IL-6 production, so that the present inventors have found that the purpose has been achieved by a hydroxamic acid derivative of formula (I), an equivalent thereof, a non-toxic salt thereof and a prodrug thereof.

An amide derivative of formula (I) of the present invention and an equivalent thereof, a non-toxic salt thereof and a prodrug thereof have not been known as a IL-6 production inhibitor at all. Furthermore, almost hydroxamic acid derivatives of formula (I-1) and equivalents of hydroxamic acid of formula (I-2), non-toxic salts thereof and prodrugs thereof are novel compounds which are not known at all.

The present invention relates to

1) an IL-6 (Interleukin-6) production inhibitor which comprises a hydroxamic acid derivative of formula (I)

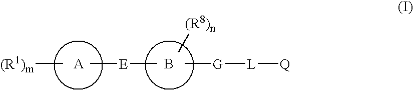

wherein, R¹ is
(a) C1-8 alkyl,
(b) C2-8 alkenyl,
(c) C2-8 alkynyl,
(d) halogen,
(e) nitro,
(f) cyano,
(g) trifluoromethyl,
(h) trifluoromethoxy,
(i) —OR²,
(j) —SR²,
(k) —NR³R⁴,
(l) —COR⁵,
(m) keto,
(n) Cycl,
(o) C1-8 alkyl substituted with —OR², —SR², —NR³R⁴, —COR⁵ or Cycl,
(p) —SO₂R¹⁰,
(q) —SOR¹⁰,
(r) —O—(C1-8 alkylene)—OR¹¹,
(s) C1-8 alkyl substituted with cyano, —CO₂R¹⁰, —SOR¹⁰ or —O—(C1-8 alkylene)—OR¹¹,
(t) —O—(C1-8 alkylene)—NR¹²R¹³,
(u) —S—(C1-8 alkylene)—NR¹³R¹³,
(v) C1-8 alkyl substituted with —O—(C1-8 alkylene)—NR¹²R¹³— or —S—(C1-8 alkylene)—NR¹²R¹³,
(w) C2-8 alkenyl substituted with —OR², —SR², —NR³R⁴, —COR⁵, Cycl, cyano, —SO₂R¹⁰, —SOR¹⁰, —O—(C1-8 alkylene)—OR¹¹, —O—(C1-8 alkylene)—NR¹²R¹³ or —S—(C1-8 alkylene)—NR¹²R¹³, or
(x) C2-8 alkynyl substituted with —OR², —SR², —NR³R⁴, —COR⁵, Cycl, cyano, —SO₂R¹⁰, —SOR¹⁰, —O—(C1-8 alkylene)—OR¹¹, —O—(C1-8 alkylene)—NR¹²R¹³ or —S—(C1-8 alkylene)—NR¹²R¹³;

wherein R² is hydrogen, C1-8 alkyl, C2-9 acyl or Cycl;

R³ and R⁴ are each independently hydrogen, C1-8 alkyl, C2-9 acyl or Cycl;

R⁵ is hydroxy, C1-8 alkyl, C1-8 alkoxy, —NR⁶R⁷ or Cycl;

R⁶ and R⁷ are each independently hydrogen, C1-8 alkyl or Cycl;

R¹⁰ is C1-8 alkyl or Cycl;

Cycl is
(a) C3-7 mono-carbocyclic ring or
(b) 5 to 7 membered mono-heterocyclic ring containing 1 to 4 nitrogen atom(s), one oxygen atom and/or one sulfur atom;

R¹¹ is hydrogen, C1-8 alkyl, C2-9 acyl or Cycl;

R¹² and R¹³ are each independently hydrogen, C1-C8 alkyl, C2-9 or Cycl;

m is 0 or an integer or 1 to 5;

A is
(a) bond,
(b) C3-15 mono-, bi- or tri-carbocyclic ring or
(c) 5 to 18 membered mono-, bi- or tri-heterocyclic ring containing 1 to 4 nitrogen atom(s), 1 to 2 oxygen atom(s) and/or 1 to 2 sulfur atom(s);

E is
(a) bond,
(b) C1-8 alkylene,
(c) C2-8 alkylene,
(d) C2-8 alkynylene,
(e) —O—,
(f) —SO₂NH—,
(g) —NHSO₂—,
(h) —CONH— or
(i) —NHCO—,
   when E is (b) to (d), one saturated carbon atom in the alkylene, alkenylene or alkynylene is optionally displaced by one oxygen atom;

B is
(a) bond,
(b) C5-15 mono-, bi- or tri-carbocyclic ring or
(c) 5 to 18 membered mono-, bi- or tri-heterocyclic ring containing 1 to 4 nitrogen atom(s), 1 to 2 oxygen atom(s) and/or 1 to 2 sulfur atom(s);

R⁸ is
(a) C1-8 alkyl,
(b) C1-8 alkoxy,
(c) halogen,
(d) nitro,
(e) cyano,
(f) trifluoromethyl,
(g) trifluoromethoxy,
(h) hydroxy or
(i) C1-8 alkyl substituted with hydroxy,
   when E is bond, R¹ and R⁸ are taken together to be C1-4 alkylene optionally,

n is 0 or an integer of 1 to 5;

G is
(a) bond,
(b) —NR²⁰CO—, wherein R²⁰ is hydrogen or C1-4 alkyl,
(c) —CONR²⁰—, wherein R²⁰ has the same meaning as defined hereinbefore,
(d) —O—,
(e) —S—,
(f) —SO—,
(g) —SO₂—,
(h) —SO₂NR²⁰—, wherein R²⁰ has the same meaning as defined hereinbefore,
(i) —CO—,
(j) —(C1-4 alkylene)—NR²³—, wherein R²³ is hydrogen, C1-8 alkyl or C1-4 alkoxycarbonyl,
(k) —(C1-4 alkylene)—OC(O)NH—,

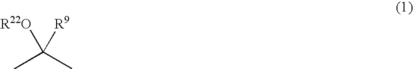

wherein R⁹ is hydrogen, hydroxy, C1-8 alkyl, C2-8 alkenyl, C2-8 alkynyl or C1-8 alkoxy, wherein the C1-8 alkyl, C2-8 alkenyl, C2-8 alkynyl or C1-8 alkoxy is optionally substituted with Cycl or C1-8 alkoxy; and R²² is hydrogen, C1-8 alkyl, C2-8 alkenyl, C2-8 alkynl, C1-8 alkyl substituted with C1-8 alkoxy, C2-8 alkenyl substituted with C1-8 alkoxy, C2-8 alkynyl substituted with C1-8 alkoxy or C2-8 alkoxyalkyl substituted with Cycl,

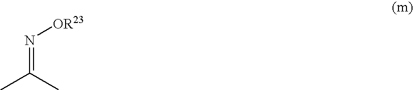

wherein R²³ is hydrogen, C1-8 alkyl, C2-8 alkenyl, C2-8 alkynyl or C2-8 alkoxyalkyl,

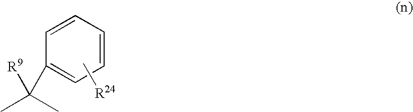

wherein R²⁴ has the same meaning as R¹, R⁹ has the same meaning as defined hereinbefore,

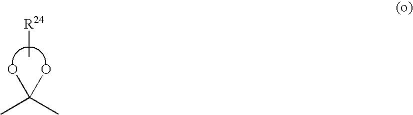

wherein 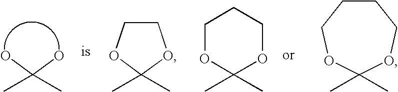
R²⁴ has the same meaning as defined hereinbefore, or

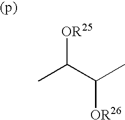

wherein R²⁵ and R²⁶ are each independently hydrogen, C1-8 alkyl, C2-8 alkenyl, C2-8 alkynyl or C2-8 alkoxyalkyl;

L is
(a) C1-8 alkylene,
(b) C2-8 alkenylene,
(c) C2-8 alkynylene,
(d) -(C2-8 alkenylene)-(C2-8 alkynylene)- or
(e) -(C2-8 alkynylene)-(C2-8 alkenylene)-
   when L is (a) to (e), 1 or 2 saturated carbon atom(s) in the alkylene, alkenylene or alkynylene is optionally displaced by 1 or 2 —CONH—, —NHCO—, —CO—, —S—, —SO—, —SO₂—, —O—, —SO₂NH—, —NHSO₂—, phenylene, C3-8 cycloalkylene or thienylene, wherein the alkylene, alkenylene or alkynylene is optionally substituted with following substituents:
(a) C1-8 alkoxy,
(b) hydroxy,
(c) C1-4 alkoxy substituted with C1-4 alkoxy,
(d) Cycl, or
(e) Cycl substituted with C1-4 alkyl or C1-4 alkoxy;

Q is Q¹ or Q².

Q¹ is

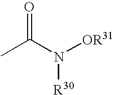

wherein R³⁰ is hydrogen or C1-8 alkyl, R³¹ is hydrogen, C1-8 or C2-8 alkoxyalkyl;

Q² is 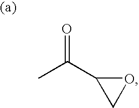
(b) —SR³²

wherein R³² is hydrogen, C1-8 alkyl or —C(O)—C1-8 alkyl,

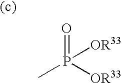

wherein R³³ is hydrogen or C1-4 alkyl, or
(d) —C(C)NR³⁴R³⁵

wherein R³⁴ is hydrogen or C1-8 alkyl, R³⁵ is hydrogen, C1-8 alkyl or NR³⁶R³⁷, wherein R³⁶ and R³⁷ are each independently hydrogen or C1-8 alkyl; and

wherein
(1) when G is

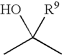

wherein R⁹ has the same meaning as defined hereinbefore, L is non-substituted tetramethylene and E is bond, —CH═CH— or —CH≡CH—, then Q is not Q¹, and
(2) A, E and B are not bond at same time,

an equivalent thereof, a non-toxic salt thereof or a prodrug thereof, as an active ingredient,
2) a novel hydroxamic acid derivative of formula (I-1) 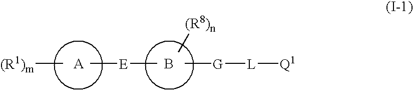
   wherein all the symbols have the same meanings as defined hereinbefore,
   a non-toxic salt thereof or a prodrug thereof,

3) a novel equivalent of hydroxamic acid derivative of formula (1-2) 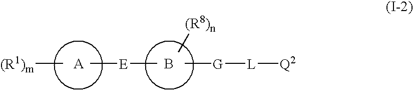
wherein all the symbols have the same meanings as defined hereinbefore, or
a non-toxic salt thereof, and

4) a process for preparation thereof.

### DETAILED EXPLANATION OF THE INVENTION

Unless otherwise specified, all isomers are included in the present invention. For example, alkyl, alkenyl, alkynyl, alkoxy, alkylene alkenylene and alkynylene groups include straight or branched ones. In addition, isomers on double bond, ring, fused ring (E-, Z-, cis-, trans-isomer), isomers generated from asymmetric carbon atom(s) (R-, S-, α-, β-isomer, enantiomer, diastereomer), optically active isomers (D-, L-, d-, l-, (+)-, (+)-isomer), polar compounds generated by chromatographic separation (more polar compound, less polar compound), equilibrium compounds, mixtures thereof at voluntary ratios and racemic mixtures are also included in the present invention.

In the present invention, C1-4 alkyl means methyl, ethyl, propyl, butyl and the isomers thereof.

In the present invention, C1-8 alkyl means methyl, ethyl, propyl, butyl, pentyl, hexyl, heptyl, octyl and the isomers thereof.

In the present invention, C2-8 alkenyl means C2-8 alkenyl which has 1 to 3 double bond(s). Specifically it means ethenyl (vinyl), propenyl, butenyl, butadienyl, pentenyl, pentadienyl, hexenyl, hexadienyl, hexatrienyl, heptenyl, heptadienyl, heptatrienyl, octenyl, octadienyl, octatrienyl and the isomers thereof.

In the present invention, C2-8 alkynyl means C2-8 alkynyl which has 1 to 3 triple bond(s). Specifically it means ethynyl, propynl, butynyl, butadiynyl, pentynyl, pentadiynyl, hexynyl, hexadiynyl, hexatriynyl, heptynyl, heptadiynyl, heptatriynyl, octynyl, octadiynyl, octatriynyl and the isomers thereof.

In the present invention, halogen means fluoride, chloride, bromide and iodide.

In the present invention, C1-4 alkylene means methylene, ethylene, trimethylene, tetramethylene and the isomers thereof.

In the present invention, C1-8 alkylene means methylene, ethylene, trimethylene, tetramethylene, pentamethylene, hexamethylene, heptamethylene, octamethylene and isomers thereof.

In the present invention, C2-4 alkenylene means C2-4 alkenylene which has 1 to 2 double bond(s). Specifically it means ethenylene (vinylene), propenylene, butenylene, butadienylene and the isomers thereof.

In the present invention, C2-8 alkenylene means C2-8 alkenylene which has 1 to 2 double bond(s). Specifically it means ethenylene (vinylene), propenylene, butenylene, butadienylene, pentenylene, pentadienylene, hexenylene, hexadienylene, heptenylene, heptadienylene, octenylene, octadienylene and the isomers thereof.

In the present invention, C2-4 alkynylene means C2-8 alkenylene which has 1 to 2 triple bond(s). Specifically it means ethynylene, propynylene, butynylene, butadiynylene and the isomers thereof.

In the present invention, C2-8 alkynylene means C2-8 alkenylene which has 1 to 2 triple bond(s). Specifically it means ethynylene, propynylene, butynylene, butadiynylene, pentynylene, pentadiynylene, hexynylene, hexadiynylene, heptynylene, heptadiynylene, octynylene, octadiynylene and the isomers thereof.

In the present invention, C2-9 acyl means ethanol (acetyl), propanoyl (propionyl), butanoyl (butyryl), pentanoyl (valeryl), hexanoyl, heptanoyl, octanoyl, nonanoyl and the isomers thereof.

In the present invention, C1-4 alkoxy means methoxy, ethoxy, propoxy, butoxy and the isomers thereof.

In the present invention, C1-8 alkoxy means methoxy, ethoxy, propoxy, butoxy, pentyloxy, hexyloxy, heptyloxy, octyloxy and the isomers thereof.

In the present invention, C2-8 alkoxyalkyl means C2-9 alkyl whose not-terminal carbon(s) is/are displaced by oxygen atom(s) and the isomers thereof. For example, methoxymethyl, ethoxymethyl, propoxymethyl, butoxymethyl, pentyloxymethyl, hexyloxymethyl, heptyloxymethyl, methoxyethyl, ethoxyethyl, propoxyethyl, butoxyethyl, pentyloxyethyl, hexyloxyethyl, methoxypropyl, ethoxypropyl, propoxypropyl, butoxypropyl, pentyloxypropyl, methoxybutyl, ethoxybutyl, propoxybutyl, butoxybutyl, methoxypentyl, ethoxypentyl, propoxypentyl, methoxyhexyl, ethoxyhexyl, methoxyheptyl and the isomers thereof.

In the present invention, C1-4 alkoxycarbonyl means methoxycarbonyl, ethoxycarbonyl, propoxycarbonyl, butoxycarbonyl and the isomers thereof.

In the present invention, C3-8 cycloalkyl means cyclopropyl, cyclobutyl, cylcopentyl, cyclohexyl, cycloheptyl and cyclooctyl.

In the present invention, phenylene means benzene ring which has two connectable bonds, i.e.,

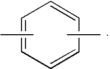

In the present invention, thienylene means thiophene ring which has two connectable bonds, i.e.,

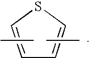

In the present invention, C3-8 cycloalkylene means cyclopropyl, cyclobutyl, cyclopentyl, cyclohexyl, cyclopentyl and cyclooctyl ring which have two connectable bonds, i.e.,

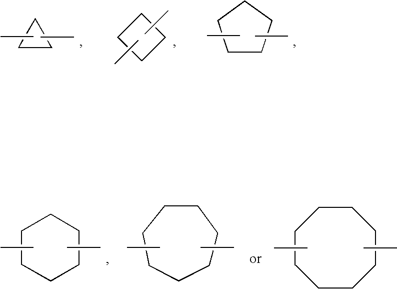

In the present invention, C3-7 mono-carbocyclic ring means C3-7 mono-aromatic carbocyclic ring, partially saturated carbocyclic ring thereof and fully saturated carbocyclic ring thereof. In includes, for example, cyclopropane, cyclobutane, cyclopentane, cyclohexane, cycloheptane, cyclopentene, cyclohexene, cycloheptene, cyclopentadiene, cyclohexadiene, cycloheptadiene, benzene ring, etc.

In the present invention, 5 to 7 membered mono-heterocycli ring containing 1 to 4 nitrogen atom(s), 1 oxygen atom and/or 1 sulfur atom means 5 to 7 membered mono-heterocyclic aryl containing 1 to 4 nitrogen atom(s), 1 oxygen atom and/or 1 sulfur atom and partially or fully saturated one. It includes, for example, pyrrole, imidazole, pyrazole, triazole, tetrazole, pyridine, pyrazine, pyrimidine, pyridazine, azepine, diazepine, furan, pyran, oxepine, oxazepine, thiophene, thianin (thiopyran), thiepine, oxazole, isoxazole, thiazole, isothiazole, oxadiazole, oxazine, oxadiazine, oxazepine, oxadiazepine, thiadiazole, thiazine, thiadiazine, thiazepine, thiadiazepine, pyrroline, pyrrolidine, imidazoline, imidazolidine, pyrazoline, pyrazolidine, triazoline, triazolidine, tetrazoline, tetrazolidine, dihydropyridine, dihydropyrimidine, dihyropyridazine, piperidine, piperazine, tetrahydropyrimidine, tetrahydropyridazine, dihydroazepine, dihydrodiazepine, tetrahydroazepine, tetrahydrodiazepine, dihydrofuran, tetrahydrofuran, dihydropyran, tetrahydropyran, dihydrothiophene, tetrahydrothiophene, dihydrothiain (dihydrothiopyran), tetrahydrothiain (tetrahydrothiopyran), dihydrooxazole, tetrahydrooxazole, dihyroisoxazole, tetrahydroisoxazole, dihydrothiazole, tetrahydrothiazole, dihydroisothiazole, tetrahydroisothiazole, tetrahydrooxadiazole, tetrahydrooxazine, tetrahydrooxadiazine, tetrahydrooxazepine, tetrahydrooxadiazepine, tetrahydrothiadiazole, tetrahydrothiazine, tetrahydrothiadiazine, tetrahydrothiazepine, tetrahydrothiadiazepine, morpholine, thiomorpholine, etc.

In the present invention, C3-15 mono-, bi- or tri-carbocyclic ring means C3-15 mono-, bi- and tri-aromatic carbocyclic ring, partially or fully saturated one. It includes, for example, cyclopropane, cyclobutane, cyclopentane, cyclohexane, cycloheptane, cyclopentene, cyclohexene, cycloheptene, cyclopentadiene, cyclohexadiene, cycloheptadiene, benzene, pentalene, indene, naphthalene, azulene, fluorene, phenanthrene, anthracene, acenaphthylene, biphenylene, perhydropentalene, perhydroindene, dihydronaphthalene, tetrahydronaphthalene, perhydronaphthalene, perhydroazulene, perhydrofluorene, perhydrophenanthrene, perhydroanthracene, perhydroacenaphthylene, perhydrobiphenylene, etc.

In the present invention, 5 to 18 membered mono-, bi- or tri-heterocyclic ring containing 1 to 4 nitrogen atom(s), 1 to 2 sulfur atom(s) means 5 to 18 membered mono-, bi- or tri-heterocyclic aryl containing 1 to 4 nitrogen atom(s), 1 to 2 oxygen and/or 1 to 2 sulfur atom(s) and partially or fully saturated one. It includes, for example, pyrrole, imidazole, pyrazole, triazole, tetrazole, pyridine, pyrazine, pyrimidine, pyridazine, azepine, diazepine, furan, pyran, oxepine, oxazepine, thiophene, thiain (thiopyran), thiepine, oxazole, isoxazole, thiazole, isothiazole, oxadiazole, oxazine, oxadiazine, oxazepine, oxadiazepine, thiadiazole, thiazine, thiadiazine, thiazepine, thiadiazepine, indole, isoindole, benzofuran, isobenzofuran, benzothiophene, isobenzothiophene, indazole, quinoline, isoquinoline, phthalazine, naphthyridine, quinoxaline, quinazolien, cinnoline, benzoxazole, benzothiazole, benzimidazole, carbazole, acridine, dibenzofuran, dibenzothiophene, pyrroline, pyrrolidine, imidazoline, imidazolidine, pyrazoline, pyrazolidine, triazoline, triazolidine, tetrazoline, tetrazolidine, dihydropyridine, dihydropyrimidine, dihydropyridazine, piperidine, piperazine, tetrahydropyrimidine, tetrahydropyridazine, dihydroazepine, dihydrodiazepine, tetrahydroazepine, tetrahydrodiazepine, dihydrofuran, tetrahydrofuran, dihydropyran, tetrahydropyran, dihydrothiophene, tetrahydrothiophene, dihydrothiain (dihydrothiopyran), tetrahydrothiain (tetrahydrothiopyran), dihydrooxazole, tetrahydrooxazole, dihydroisoxazole, tetrahydroisoxazole, dihydrothiazole, tetrahydrothiazole, dihydroisothiazole, tetrahydroisothiazole, tetrahydrooxadiazole, tetrahydrooxazine, tetrahydrooxadiazine, tetrahydrooxazepine, tetrahydrooxadiazepine, tetrahydrothiadiazole, tetrahydrothiazine, tetrahydrothiadiazine, tetrahydrothiazepine, tetrahydrothiadiazepine, morpholine, thiomorpholine, indoline, isoindoline, dihydrobenzofuran, perhydrobenzofuran, dihydroisobenzofuran, perhydroisobenzofuran, dihydrobenzothiophene, perhydrobenzothiophene, dihydroisobenzothiophene, perhydroisobenzothiophene, dihydroindazole, perhydroindazole, dihydroquinoline, tetrahydroquinoline, perhydroquinoline, dihydroisoquinoline, tetrahydroisoquinoline, perhydroisoquinoline, dihydrophthalazine, tetrahydrophthalazine, perhydrophthalazine, dihydronaphthyridine, tetrahydronaphthyridine, perhydronaphthyridine, dihydroquinoxaline, tetrahydroquinoxaline, perhydroquinoxaline, dihydroquinazoline, tetrahydroquinazoline, perhydroquinazoline, dihydrocinnoline, tetrahydrocinnoline, perhydrocinnoline, dihydrobenzoxazole, perhydrobenzoxazole, dihydrobenzothiazole, perhydrobenzothiazole, dihydrobenzimidazole, perhydrobenzimidazole, benzoxazepine, benzoxadiazepine, benzothiazepine, benzothiadiazepine, benzazepine, benzodiazepine, indroxoazepine, indrotetrahydrooxazepine, indroxadiazepine, indrotetrahydroxadiazepine, indrothiazepine, indrotetrahydrothiazepine, indrothiadiazepine, indrotetrahydrothiadiazepine, indroazepine, indrotetrahydroazepine, indrodiazepine, indrotetrahydrodiazepine, benzofurazan, benzothiadiazole, benzotriazole, imidazothiazole, dihydrocarbazole, tetrahydrocarbazole, perhydrocarbazole, dihydroacridine, tetrahydroacridine, perhydroacridine, dihydrodibenzofuran, dihydrodibenzothiophene, tetrahydrodibenzofuran, tetrahydrodibenzothiophene, perhydrodibenzofuran, perhydrodibenzothiophene, 1,3-dioxaindan, 1,4-dioxotetrahydronaphthalene ring, etc.

Among the compounds of the present invention, the equivalent of the hydroxamic acid is the compound whose substituent shown as 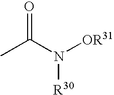
(wherein all the symbols have the same meanings as defined hereinbefore) is displaced by the substituent as shown as 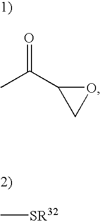
(wherein R³² has the same meaning as defined hereinbefore), 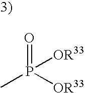
(wherein all the symbols have the same meanings as defined hereinbefore) or
4) —C(O)NR³⁴R³⁵
   (wherein all the symbols have the same meanings as defined hereinbefore), or the non-toxic salt thereof.

In the present invention, unless otherwise specified, as will be apparent to those skilled in the art, a symbol [symbol] represents bonding to back of the paper (that is, α-configuration), [symbol] represents bonding to front of the paper (that is, β-configuration), [symbol] represents α-, β- or mixture thereof and [symbol] represents mixture of α-configuration and β-configuration.

The compound of the present invention may be converted into the corresponding non-toxic salt by conventional methods.

Non-toxic salts of the compounds of the present invention include all pharmaceutically acceptable salts, and water-soluble salts are preferred.

Non-toxic salts of the compounds of the present invention, for example, include: salts of alkali metals (e.g. potassium, sodium, lithium, etc.), salts of alkaline earth metals (e.g. calcium, magnesium, etc.), ammonium salts (e.g. tetramethylammonium salt, tetrabutylammonium salt, etc.), salts of organic amines (e.g. triethylamine, methylamine, dimethylamine, cyclopentylamine, benzylamine, phenethylamine, piperidine, monoethanolamine, diethanolamine, tris(hydroxymethyl)methylamine, lysine, arginine, N-methyl-D-glucamine, etc.) and acid addition salts (salts of inorganic acids (e.g. hydrochloride, hydrobromide, hydroiodide, sulfate, phosphate, nitrate, etc.), salts of organic acids (e.g. acetate, trifluoroacetate, lactate, tartrate, oxalate, fumarate, maleate, benzoate, citrate, methanesulfonate, ethanesulfonate, benzenesulfonate, toluenesulfonate, isethionate, glucuronate, gluconate, etc.), etc.).

Non-toxic salts of the compounds of the present invention includes solvates thereof or solvates of the salts of alkali metals, salts of alkaline earth metals, ammonium salts, salts of organic amines and acid addition salts of the compounds of the present invention.

Non-toxic and water-soluble solvates are preferred. Solvates of the compounds of the present invention, for example, include: hydrates, solvates of the alcohols (ethanol etc.), etc.

Besides, the prodrug of the invention means the compound of formula (I) converted 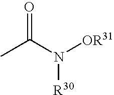
part thereof to

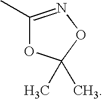

In the present invention, R¹ is, preferably, C1-8 alkyl, halogen, C1-8 alkoxy, cyano, —NR³R⁴, —OR², —SR², —COR⁵ or C1-8 alkyl substituted with —NR³R⁴, —OR² or Cycl. More preferably, R¹ is C1-4 alkyl, halogen, cyano, C1-4 alkoxy or substituted C1-4 alkyl.

In the present invention, A is, preferably, bond, C3-10 mono-, bi-carbocyclic ring or 5 to 10 membered mono-, bi-heterocyclic ring containing 1 to 4 nitrogen atom(s), 1 to 2 oxygen atom(s) and/or 1 to 2 sulfur atom(s). More preferably, A is bond, C5-7 mono-carboxylic ring or 5 to 10 membered mono-, bi-heterocyclic ring containing 1 to 4 nitrogen atom(s), 1 to 2 oxygen atom(s) and/or 1 to 2 sulfur atom(s).

Especially, A is, more preferably, bond, benzene, cyclohexane, pyridine, morpholine, naphthalene, benzoxazole, benzothiazole, benzimidazole, benzothiophene or benzofuran ring.

In the present invention, E is, preferably, bond, C1-4 alkylene, C2-4 alkenylene, C2-4 alkynylene (one saturated carbon atom in the alkylene, alkenylene, alkynylene is optionally displaced by oxygen atom), —NHCO—, —SO₂NH— or oxygen. More preferably, E is bond, C1-3 alkylene, C2-3 alkenylene, C2-4 alkynylene whose saturated carbon atom is optionally displaced by oxygen atom, —NHCO—, —CONH—, —SO₂NH— or oxygen.

In the present invention, B is, preferably, C5-10 mono- or bi-carbocyclic ring or 5 to 15 membered mono- or bi-heterocyclic ring containing 1 to 4 nitrogen atom(s), 1 to 2 oxygen atom(s) and/or 1 to 2 sulfur atom(s). More preferably, B is C5-10 mono- or bi-carbocyclic ring or 5 to 10 membered mono- or bi-heterocyclic ring containing 1 to 2 nitrogen atom(s), 1 oxygen atom and/or 1 sulfur atom.

Especially, B is, more preferably, benzene, cyclohexane, cyclohexene, naphthalene, pyridine, pyrimidine, thiophene, benzofuran, benzothiophene or benzoxazole ring.

In the present invention, all G defined hereinbefore is preferable. Especially, G is, more preferably, bond, —CONR²⁰—, —SO—, —SO₂—, —SO₂NR²⁰—, —CO—, —(C1-4 alkylene)—NR²³,

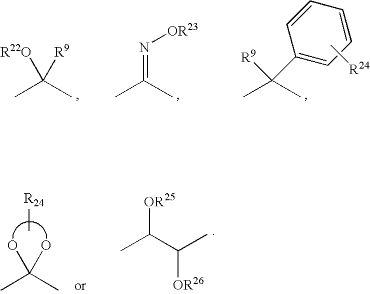

In the present invention, L is, preferably, C1-8 alkylene, C2-8 alkenylene or C2-8 alkynylene. More preferably, L is C2-6 alkylene, C2-6 alkenylene or C2-6 alkynylene.

One or two saturated carbon atom(s) in the preferred alkylene, alkenylene or alkynylene is optionally displaced by —CONH—, —NHCO—, —S—, —SO—, —SO₂—, —O—, —SO₂NH—, —NHSO₂—, phenylene, C3-8 cycloalkylene or thienylene. The alkylene, alkenylene or alkynylene is optionally substituted with Cycl.

In the present invention, Q is, preferably, Q¹ or Q². Especially, Q¹ is more preferable.

Besides the compounds described in Examples hereinafter, a preferable compound is specifically the compound of formula (I-A1) 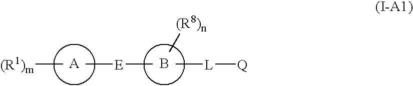
(wherein all the symbols have the same meanings as defined hereinbefore), formula (I-A2) 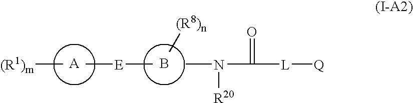
(wherein all the symbols have the same meanings as defined hereinbefore), formula (I-A3) 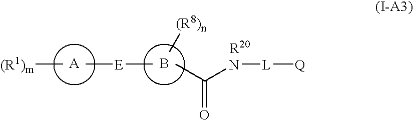
(wherein all the symbols have the same meanings as defined hereinbefore), formula (I-A4) 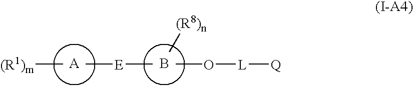
(wherein all the symbols have the same meanings as defined hereinbefore), formula (I-A5) 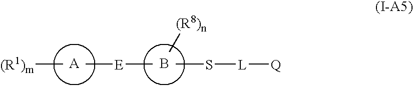
(wherein all the symbols have the same meanings as defined hereinbefore), formula (I-A6) 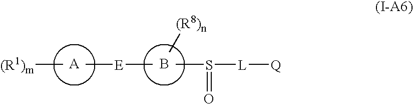
(wherein all the symbols have the same meanings as defined hereinbefore), formula (I-A7) 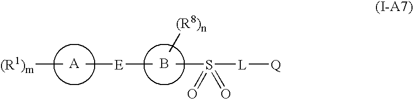
(wherein all the symbols have the same meanings as defined hereinbefore), formula (I-A8) 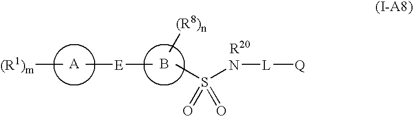
(wherein all the symbols have the same meanings as defined hereinbefore), formula (I-A9) 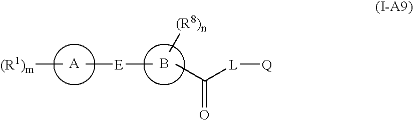
(wherein all the symbols have the same meanings as defined hereinbefore), formula (I-A10) 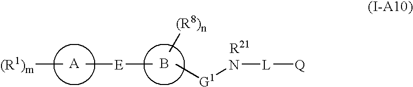
(wherein G¹ is C1-4 alkylene, and other symbols have the same meanings as defined hereinbefore), formula (I-A11) 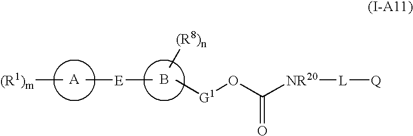
(wherein all the symbols have the same meanings as defined hereinbefore), formula (I-A12) 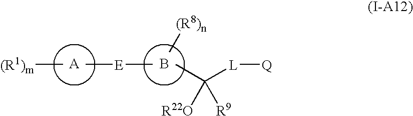
(wherein all the symbols have the same meanings as defined hereinbefore), formula (I-A13) 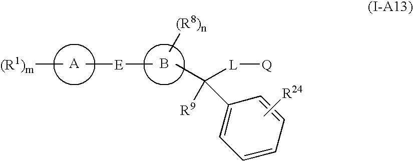
(wherein all the symbols have the same meanings as defined hereinbefore), formula (I-A14) 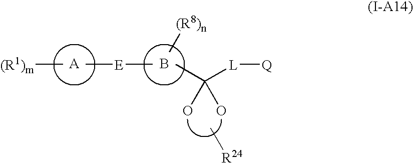
(wherein all the symbols have the same meanings as defined hereinbefore), or formula (I-A15) 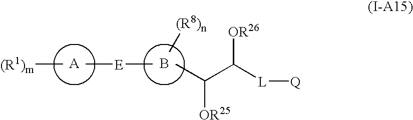
(wherein all the symbols have the same meanings as defined hereinbefore).

The compounds shown in the following table 1 to 5 are also preferable.

TABLE 1

| | |
|---|---|
| | (I-A3-1) |
| 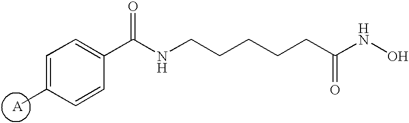 | |

| No. | A |
|---|---|
| 1 | 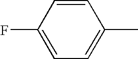 |
| 2 | 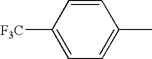 |
| 3 | 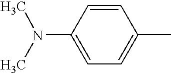 |
| 4 | 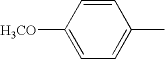 |
| 5 | 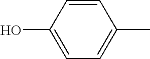 |
| 6 | 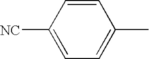 |
| 7 | 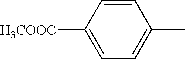 |
| 8 | 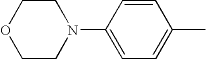 |
| 9 | 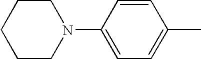 |
| 10 | 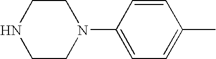 |
| 11 | 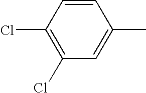 |
| 12 | 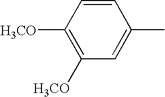 |
| 13 | 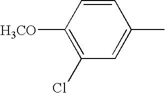 |
| 14 | 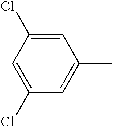 |
| 15 | 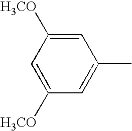 |
| 16 | 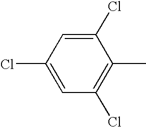 |
| 17 | 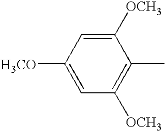 |
| 18 | 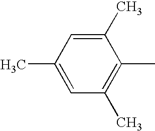 |

TABLE 2

| | |
|---|---|
| | (I-A3-2) |
| 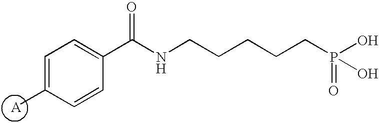 | |

| No. | A |
|---|---|
| 1 | 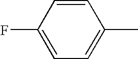 |
| 2 | 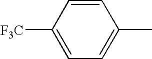 |
| 3 | 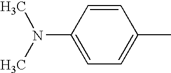 |
| 4 | 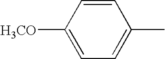 |
| 5 | 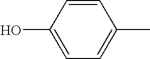 |
| 6 | 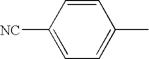 |
| 7 | 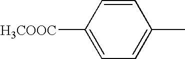 |
| 8 | 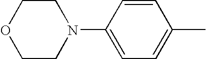 |
| 9 | 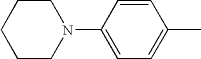 |
| 10 | 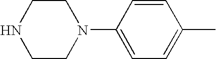 |
| 11 | 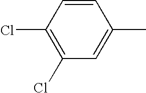 |
| 12 | 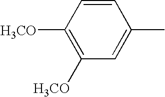 |
| 13 | 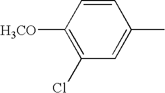 |
| 14 | 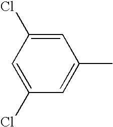 |
| 15 | 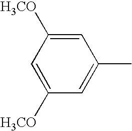 |
| 16 | 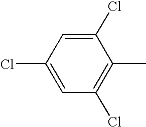 |
| 17 | 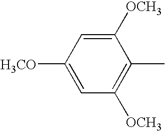 |
| 18 | 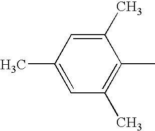 |

TABLE 3

| | |
|---|---|
| | (I-A3-3) |
| 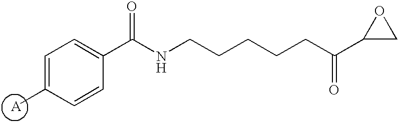 | |

| No. | A |
|---|---|
| 1 | 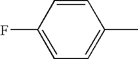 |
| 2 | 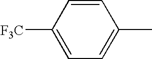 |
| 3 | 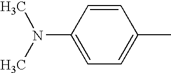 |
| 4 | 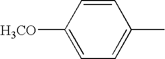 |
| 5 | 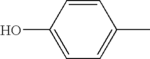 |
| 6 | 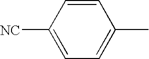 |
| 7 | 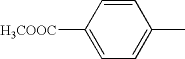 |
| 8 | 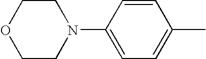 |
| 9 | 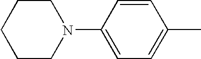 |
| 10 | 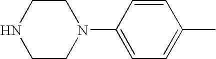 |
| 11 | 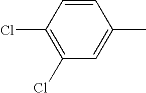 |
| 12 | 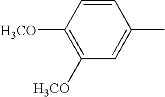 |
| 13 | 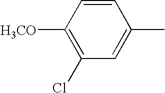 |
| 14 | 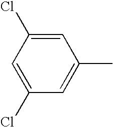 |
| 15 | 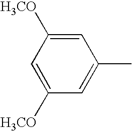 |
| 16 | 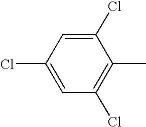 |
| 17 | 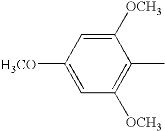 |
| 18 | 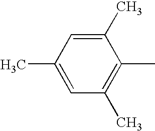 |

TABLE 4

| | |
|---|---|
| | (I-A3-4) |
| 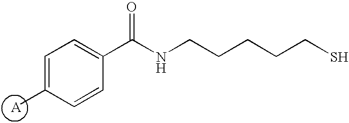 | |

| No. | A |
|---|---|
| 1 | 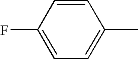 |
| 2 | 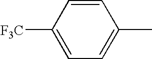 |
| 3 | 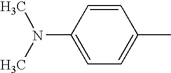 |
| 4 | 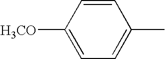 |
| 5 | 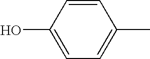 |
| 6 | 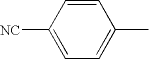 |
| 7 | 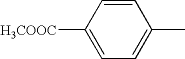 |
| 8 | 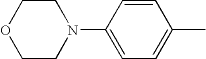 |
| 9 | 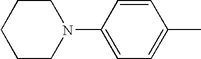 |
| 10 | 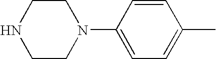 |
| 11 | 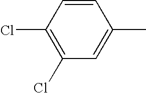 |
| 12 | 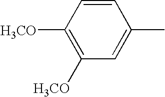 |
| 13 | 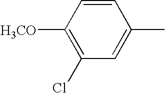 |
| 14 | 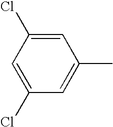 |
| 15 | 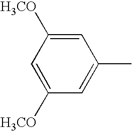 |
| 16 | 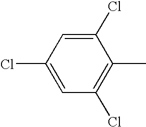 |
| 17 | 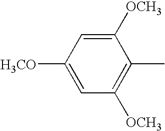 |
| 18 | 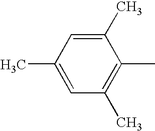 |

TABLE 5

| | |
|---|---|
| | (I-A3-5) |
| 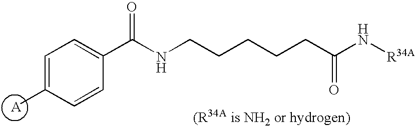 | |

| No. | A |
|---|---|
| 1 | 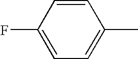 |
| 2 | 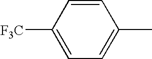 |
| 3 | 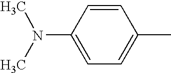 |
| 4 | 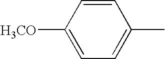 |
| 5 | 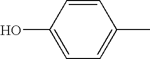 |
| 6 | 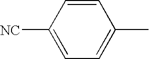 |
| 7 | 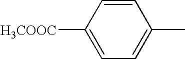 |
| 8 | 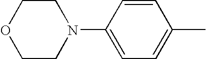 |
| 9 | 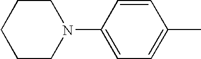 |
| 10 | 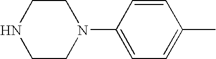 |
| 11 | 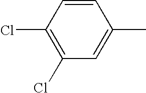 |
| 12 | 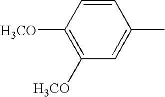 |
| 13 | 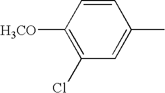 |
| 14 | 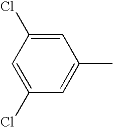 |
| 15 | 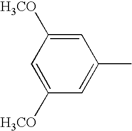 |
| 16 | 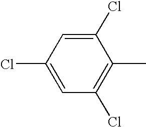 |
| 17 | 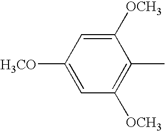 |
| 18 | 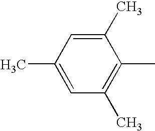 |

A process for the preparation of the compound of the present invention.

The compounds of formula (I) of the present invention may be prepared by the following methods or the methods described in Examples.
(1) Among the compounds of formula (I) in the present invention, a hydroxamic acid derivative of formula (I-1) 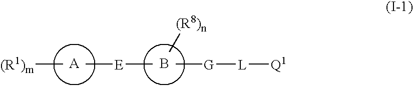
   (wherein all the symbols have the same meanings as defined hereinbefore), a non-toxic salt thereof or a prodrug thereof may be prepared by the following methods or the methods described in Examples.
(1-a) Among the compounds of formula (I-1), the compound that Q¹ is 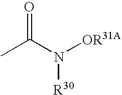
   (wherein R^{31A} has the same meaning of R³¹, but does not represent hydrogen atom; R³⁰ has the same meaning as defined hereinbefore)
   and any of R¹, A, B, R⁸ or G does not represent the group which contains amino, carboxyl nor thiol group, i.e. the compound of formula (I-1a) 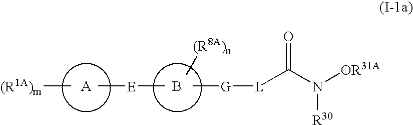
   (wherein, R^{1A}, A^{A}, B^{A}, R^{8A}, G^{A} and R^{31A} are each same meanings of R¹, A, B, R⁸, G and R³¹, but any of them does not represent the group which contains amino, carboxyl nor thiol group, and the other symbols have the same meanings as defined hereinbefore), may be prepared by amidation of the carboxylic acid derivative of formula (II) 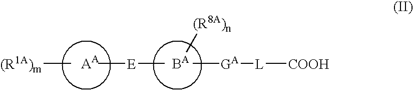
   (wherein all the symbols have the same meanings as defined hereinbefore) with the hydroxylamine derivative of formula (III) 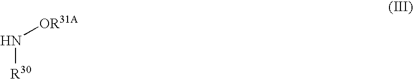
   (wherein all the symbols have the same meanings as defined hereinbefore).

The amidation is know. For example, it may be carried out
(1) by the method with using acid halide,
(2) by the method with using mixed acid anhydride,
(3) by the method with using condensing agent, etc.

Concrete description of these methods are as follows:

(1) The method with acid halide may be carried out, for example; a carboxylic acid is reacted with an acid halide (oxalyl, chloride, thionyl chloride, etc.) in an organic solvent (chloroform, methylene chloride, diethyl ether, tetrahydrofuran, etc.) or without solvents at from −20° C. to refluxing temperature to give a corresponding acid halide. The obtained acid halide and an amine are reacted in an organic solvent (chloroform, methylene chloride, diethyl ether, tetrahydrofuran, etc.) in the presence of tertiary amine (pyridine, triethylamine, dimethylamine, dimethylaniline, dimethylaminopyridine, etc.) at from 0 to 40° C. This reaction may also be carried out by the reaction with an amine and a acid halide in an organic solvent (dioxane, tetrahydrofuran, etc.) with an aqueous alkali solution (solution of sodium bicarbonate or solution of sodium hydroxide, etc.) at from 0 to 40° C.

(2) The method with mixed acid anhydride may be carried out, for example; a carboxylic acid is reacted with an acid halide (pivaloyl chloride etc.) or an acid derivative (ethyl chloroformate, isobutyl chloroformate, etc.) in an organic solvent (chloroform, methylene chloride, diethyl ether, tetrahydrofuran, etc.) or without solvents, in the presence of tertiary amine (pyridine, triethylamine, dimethylaniline, dimethylaminopylidine, etc.) at from 0 to 40° C. The obtained mixed acid anhydride is reacted with an amine in an organic solvent (chloroform, methylene chloride, diethyl ether, tetrahydrofuran, etc.) at from 0 to 40° C.

(3) The method with condensing agent may be carried out, for example; a carboxylic acid and an amine are reacted in an organic solvent (chloroform, methylene chloride, dimethylformamide, diethyl ether, tetrahydrofuran, etc.) or without solvents, in the presence or absence of tertiary amine (pyridine, triethylamine, dimethylaniline, dimethylaminopyridine, etc.) using with condensing agent (1,3-dicyclohexylcarbodiimide (DCC), 1-ethyl-3-[3-(dimethylamino)propyl]carbodiimide (EDC), 1,1′-carbonyldiimidazole (CDI), 2-chloro-1-methylpyridinium iodide, 1-propanephosphonic acid cyclic anhydride (PPA), etc.) using with or without 1-hydroxybenzotriazole (HOBt) at from 0 to 40° C.

Preferably, the above reactions of (1), (2) and (3) are carried out under an atmosphere at an inert gas (argon, nitrogen etc.) on anhydrous condition.
(1-b) Among the compounds of formula (I-1), the compound that Q¹ is 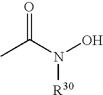
   (wherein R³⁰ has the same meaning as defined hereinbefore) and any of R¹, A, B, R⁸ or G does not represent the group which contains amino, carboxyl nor thiol group, i.e. the compound of formula (I-1b) 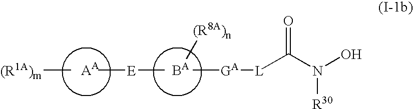
   (wherein all the symbols have the same meanings as defined hereinbefore) may be prepared by the method of (1-b-1) or (1-b-2).
(1-b-1) The compound of formula (I-1b) may be prepared by the removal of a protecting group in the hydroxamic acid of formula (I-1a).

The removal of a protecting group in the hydroxamic acid is known. For example, it may be carried out.
(1) The compound of formula (I-1b) may be prepared by the removal of a protecting group in the hydroxamic acid of formula (I-1a).

The removal of a protecting group in the hydroxamic acid is known. For example, it may be carried out
(1) by the removal of a protecting group in an alkaline condition,
(2) by the removal of a protecting group in an acidic condition,
(3) by the removal of a protecting group by hydrogenolysis, etc.

Concrete description of these methods are as follows:

(1) The removal of protecting group in an alkaline condition may be carried out, for example, in an organic solvent (methanol, tetrahydrofuran, dioxane, etc.) with hydroxide of alkaline metal (sodium hydroxide, potassium hydroxide, lithium hydroxide, etc.), hydroxide of alkaline earth metal (barium hydroxide, calcium hydroxide, etc.), carbonate (sodium carbonate, potassium carbonate, etc.) or an aqueous solution thereof or a mixture thereof at from 0 to 40° C.

(2) The removal of protecting group in an acidic condition may be carried out, for example, in an organic solvent (dichloromethane, chloroform, dioxane, ethyl acetate, anisole, etc.), an organic acid (acetic acid, trifluoroacetic acid, methanesulfonic acid, etc.) or an inorganic acid (hydrochloric acid, sulfuric acid, etc.) or a mixture thereof (hydrogen bromide/acetic acid etc.) at from 0 to 100° C.

(3) The removal of a protecting group by hydrogenolysis may be carried out, for example, in a solvent (ether (tetrahydrofuran, dioxane, dimethoxyethane, diethylether, etc.), alcohol (methanol, ethanol, etc.), benzene (benzene, toluene, etc.), ketone (acetone, methyl ethylketone, etc.), nitrile (acetonitrile, etc.), amide (dimethylformamide etc.), water, ethyl acetate, acetic acid or a mixture thereof, etc.) in the presence of a catalyst (palladium-carbon, palladium black, palladium hydroxide, platinum oxide, Raney nickel, etc.) at atmospheric or reduced pressure under an atmosphere of hydrogen or ammonium formate at from 0 to 200° C.

In the present invention, the reaction of the removal of a protecting group of a hydroxamic acid means an ordinal one which is well known to the person in the art, for example, the removal of a protecting group in an alkaline condition, the removal of a protecting group in an acidic condition or the removal of a protecting group by hydrogenolysis. The aimed compound of the present invention may be prepared easily by choice of these reactions.

As well known to the person in the art, a protecting group of a hydroxamic acid includes, for example, t-butyl, —C(CH₃)₂—OCH₃ and benzyl. In addition, such a group includes the other protecting group which is removable selectively and easily, for example, one described in T. W. Greene et al., Protective Groups in Organic Synthesis, Third Edition, Wiley-Interscience, New York, 1999.
(1-b-2) The compound of formula (I-1b) may be prepared by amidation of the compound of formula (II) and the hydroxylamine derivative of formula (IV)
   HR³⁰N—OH (IV)
   (wherein R³⁰ has the same meaning as defined hereinbefore).

The amidation may be carried out by the same procedure as described in (1-a).
(1-c) Among the compounds of formula (I-1), the compound that Q¹ is 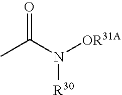
   (wherein all the symbols have the same meanings as defined hereinbefore) and that one or more of R¹, A, B, R⁸ and G represent(s) the group which contains amino, thiol or carboxyl, i.e. the compound of formula (I-1c) 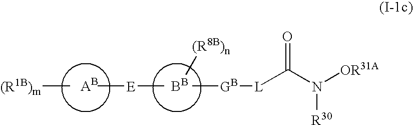
   (wherein, R^{1B}, A^{B}, B^{B}, R^{8B} and G^{B} are each same meanings of R¹, A, B, R⁸ and G, and one or more of them represent(s) amino, thiol or carboxyl; and the other symbols have the same meanings as defined hereinbefore.),
   may be prepared by the removal of a protecting group in the compound in which one or more of R^{1A}, A^{A}, B^{A}, R^{8A} and G^{A} represent(s) the group which contains amino, thio or carboxyl, among the compounds of formula (I-1a), i.e. the compound of formula (I-1a-1) 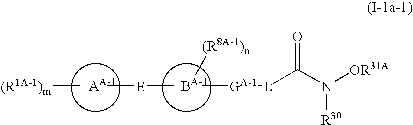
   (wherein, R^{1A-1}, A^{A-1}, B^{A-1}, R^{8A-1} and G^{A-1} are each same meanings of R^{1A}, A^{A}, B^{A}, R^{8A} and G^{A}, and one or more of them represent(s) amino, thiol or carboxyl; and the other symbols have the same meanings as defined hereinbefore).

A protecting group of amino includes, for example, benzyloxycarbonyl, allyloxycarbonyl, t-butoxycarbonyl, trifluoroacetyl, 9-fluorenylmethoxycarbonyl, etc.

A protecting group of thiol includes, for example, benzyl, methoxybenzyl, acetoamidemethyl, triphenylmethyl, acetyl, etc.

A protecting group of carboxy includes, for example, methyl, ethyl, t-butyl, benzyl, allyl, etc.

The protecting group of amino, thiol or carboxy includes the above one, in addition, the other protecting group which is removable selectively and easily, for example, one described in T. W. Greene et al., Protective Groups in Organic Synthesis, Third Edition, Wiley-Interscience, New York, 1999.

The removal of a protecting group of amino, thiol or carboxy is well known. For example, it is
(1) the removal of a protecting group in an alkaline condition,
(2) the removal of a protecting group in an acidic condition,
(3) the removal of a protecting group by hydrogenolysis,
(4) the removal of a protecting group using metal complex, etc.

The reactions of (1) to (3) may be carried out by the above method, i.e. the removal reaction of a protecting group of a hydroxamic acid described above.

Concrete description of the method of (4) is as follows:

The removal of a protecting group using metal complex may be carried out, for example, in an organic solvent (dichloromethane, dimethylformamide, tetrahydrofuran, etc.) in the presence of a trap reagent (tributyltin hydride, dimedone, etc.) and/or an organic acid (acetic acid etc.) with metal complex (tetrakis(triphenylphosphine)palladium(0) complex etc.) at from 0 to 40° C.

As well known to the person in the art, the aimed compound of present invention may be prepared easily by choice of these reactions.
(1-d) Among the compounds of formula (I-1), the compound that Q¹ is 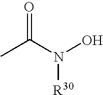
   (wherein R³⁰ has the same meaning as defined hereinbefore) and that one or more of R^{1A}, A^{A}, B^{A}, R^{8A} and G^{A} represent(s) the group which contains amino, thiol or carboxyl, i.e. the compound of formula (I-1d) 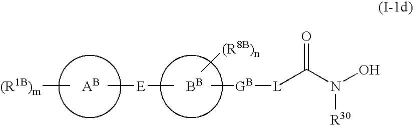
   (wherein all the symbols have the same meanings as defined hereinbefore), may be prepared by
1) the removal of the protecting group of amino, thiol, or carboxyl of the compound that one or more of R¹, A, B, R⁸ and G represent(s) the group which contains protected amino, protected thiol or protected carboxyl among the compounds of formula (I-1b), i.e. the compound of formula (I-1b-1) 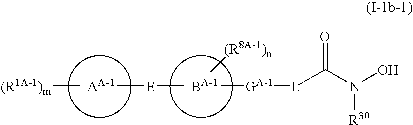
   (wherein all the symbols have the same meanings as defined hereinbefore) or
2) the removal of the protecting group of a hydroxamic acid of the compound of formula (I-1c).

The removal reaction of 1) may be carried out by the method of (1-b-1). And the removal one of 2) may be carried out by the method of (1-c).
(2) Among the compounds of formula (I) in the present invention, the equivalent of hydroxamic acid derivative of formula (I-2) 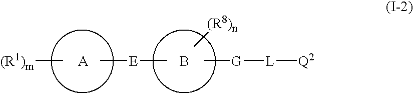
   (wherein all the symbols have the same meanings as defined hereinbefore) may be prepared by the following methods or the methods described in Examples.
(2-a) Among the compounds of formula (I-2), the epoxide derivative that Q² is 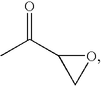
   i.e. the compound of formula (I-2a) 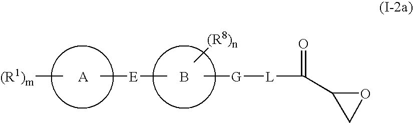
   (wherein all the symbols have the same meanings as defined hereinbefore), may be prepared by the following methods.
   (2-a-1) Among the compounds of formula (I-2a), the compound that any of R¹, A, B, R⁸ or G does not represent the group which contains amino, carboxyl nor thiol group, i.e. the compound of formula (I-2a-1) 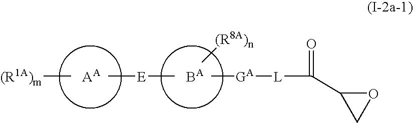
      (wherein all the symbols have the same meanings as defined hereinbefore) may be prepared by epoxidation of the compound of formula (V) 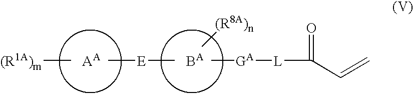
      (wherein all the symbols have the same meanings as defined hereinbefore).

The epoxidation is known. For example, it may be carried out in an inert organic solvent (ether (diethyl ether, tetrahydrofuran, etc.), halogen solvent (chloroform, dichloromethane, etc.), benzene, etc.) in the presence of base (N-benzyltrimethyl ammonium hydroxide (Triton B (registered trademark)), sodium hydrogen carbonate, potassium carbonate, etc.) and peroxide (3-chloroperbenzoic acid, t-butyl peroxide, etc.) at from −20° to 50° C.
(2-a-2) Among the compounds of formula (I-2a), the compound that one or more of R¹, A, B, R⁸ and G represent(s) the group which contains amino, thiol or carboxyl, i.e. the compound of formula (I-2a-2) 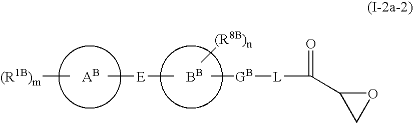
   (wherein all the symbols have the same meanings as defined hereinbefore), may be prepared by the removal of the protecting group of amino, thiol or carboxyl of the compound that one or more of R^{1A}, A^{A}, B^{A}, R^{8A} and G^{A} represent(s) the group which contains protected amino, protected thiol or protected carboxyl among the compounds of formula (I-2a-1), i.e. the compound of formula (I-2a-1-1) 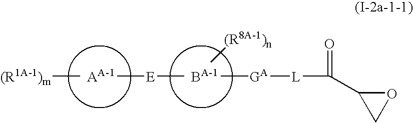
   wherein all the symbols have the same meanings as defined hereinbefore).

The removal reaction may be carried out by the method of (1-b-1).
(2-b) Among the compounds of formula (I-2), the compound that Q² is SR³² (wherein R³² has the same meaning as defined hereinbefore), i.e. the thio derivative of formula (I-2b) 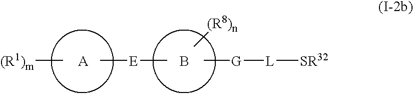
   (wherein all the symbols have the same meanings as defined hereinbefore), may be prepared by the following methods.
(2-b-1) Among the thiol derivatives of formula (I-2b), the compound that any of R¹, A, B, R⁸ or G does not represent the group which contains amino, carboxyl nor thiol group, i.e. the compound of formula (I-2b-1) 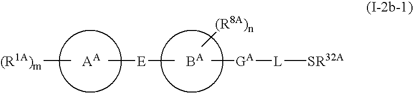
   (wherein R^{32A} is same meaning of R³², but does not represent hydrogen atom; and other symbols have the same meaning as defined hereinbefore.), may be prepared by the reaction of the compound of formula (VI) 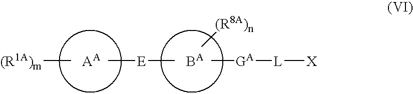
   (wherein X represents a leaving group such as hydrogen atom, p-toluenesulfonate, etc., and other symbols have the same meaning as defined hereinbefore) and the compound of formula (VII)
   R^{32A}S-G (VII)
   (wherein G represents alkali metal such as potassium, sodium, etc., and R^{32A} has the same meaning as defined hereinbefore).

The reaction may be carried out in an inert organic solvent (N,N-dimethylformamide, dimethylsulfoxide, N,N-dimethylacetamide, etc.), halogen solvent (chloroform, dichloromethane, etc.), etc.) in the presence of alkali metal's salt of thiols (potassium acetylthiolate, sodium acetylthiolate, etc.) at from 0 to 60° C.
(2-b-2) Among the thiol derivatives of formula (I-2b), the compound that one or more of R¹, A, B, R⁸ and G represent(s) the group which contains amino, thiol or carboxyl, i.e. the compound of formula (I-2b-2) 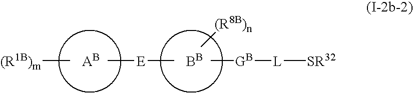
   (wherein all the symbols have the same meanings as defined hereinbefore), may be prepared by the removal of the protecting group of amino, thiol or carboxyl of the compound that one or more of R¹, A, B, R⁸ and G represent(s) the group which contains protected amino, protected thiol or protected carboxyl among the compounds of formula (I-2b-1), i.e. the compound of formula (I-2b-1-1) 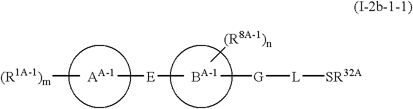
   (wherein all the symbols have the same meanings as defined hereinbefore).

The removal reaction may be carried out by the method of (1-b-1).
(2-c) Among the compounds of formula (I-2), the compound that Q² is 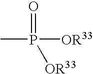
   (wherein R³³ has the same meaning as defined hereinbefore), i.e. the phosphoric acid derivative of formula (I-2c) 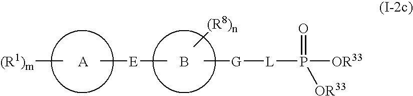
   (wherein all the symbols have the same meanings as defined hereinbefore), may be prepared by the following methods.
(2-c-1) Among the phosphoric acid derivatives of formula (I-2c), the compound that any of R¹, A, B, R⁸ or G does not represent the group which contains amino, thiol nor carboxyl group and that R³³ does not represent hydrogen atom, i.e. the compound of formula (I-2c-1) 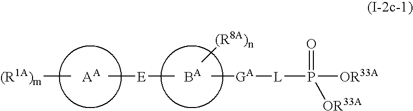
   (wherein R^{33A} is same meanings of R³³, but does not represent hydrogen atom; and other symbols have the same meaning as defined hereinbefore.), may be prepared by the reaction of the compound of formula (VI) 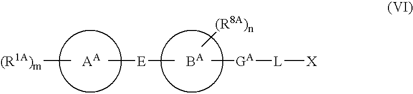
   (wherein all the symbols have the same meanings as defined hereinbefore) and the compound of formula (VIII) 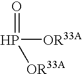
   (wherein all the symbols have the same meanings as defined hereinbefore).

This reaction may be carried out in an inert organic solvent (N,N-dimethylformamide, N,N-dimethylacetamide, dimethylsulfoxide, etc.) in the presence of base (sodium hydride, butyl lithium, etc.) at from 0 to 60° C.
(2-c-2) Among the compounds of formula (I-2c), the compound that one or more of R¹, A, B, R⁸ and G represent(s) the group which contains unprotected amino, unprotected thiol or unprotected carboxyl and that R³³ does not represent hydrogen atom, i.e. the compound of formula (I-2c-2) 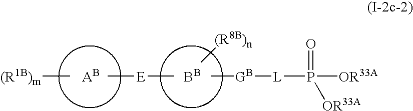
   (wherein all the symbols have the same meanings as defined hereinbefore) may be prepared by the removal of the protecting group of amino, thiol or carboxyl of the compound that one or more R^{1A-1}, A^{A-1}, B^{A-1}, R^{8A-1} and G^{A-1} represent(s) the group which contains protected amino, protected thiol or protected carboxyl among the compounds of formula (I-2c-1), i.e. the compound of formula (I-2-c-1-1) 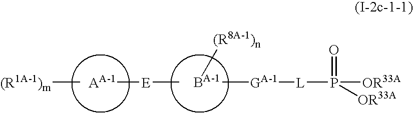
   (wherein all the symbols have the same meanings as defined hereinbefore)

The removal reaction may be carried out by the method of (1-b-1).
(2-c-3) Among the compounds of formula (I-2c), the compound that any of R¹, A, B, R⁸ or G does not represent the group which contains amino, carboxyl nor thiol group, and that R³³ does not represent hydrogen atom, i.e. the compound of formula (I-2c-3) 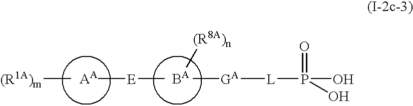
   (wherein all the symbols have the same meanings as defined hereinbefore) may be prepared by the removal of the protecting group of phosphono of the compound of formula (I-2-c-1).

The removal of protective group of phosphono may be carried out in an inert organic solvent (acetonitrile, ether (diethyl ether, tetrahydrofuran, etc.), halogen solvent (chloroform, dichloromethane, etc.), etc.) in the presence of trimethylsilyl iodide (it may be generated by using sodium iodide and trimethylsylyl chloride or trimethylsylyl bromide in situ) at from 0 to 60° C.
(2-c-4) Among the compounds of formula (I-2c), the compound that have unprotected phosphono and that one or more of R¹, A, B, R⁸ and G represent(s) the group which contains amino, thiol or carboxyl, i.e. the compound of formula (I-2c-4) 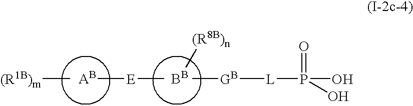
   (wherein all the symbols have the same meanings as defined hereinbefore), may be prepared by
1) the removal of the protecting group of amino, thiol or carboxyl of the compound that one or more of R^{1A}, A^{A}, B^{A}, R^{8A} and G^{A} represent(s) the group which contains protected amino, protected thiol or protected carboxyl among the compounds of formula (I-2c-3), i.e. the compound of formula (I-2c-3-1) 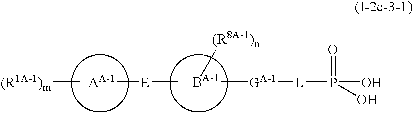
   (wherein all the symbols have the same meanings as defined hereinbefore) or
2) the removal of the protecting group of phosphono of the compound of formula (I-2c-2)

The removal reaction of 1) may be carried out by the method of (1-b-1). And the removal of one of 2) may be carried out by the method of (2-c-3).
(2-d) Among the compounds of formula (I-2), the compound that Q² is 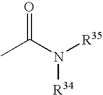
   (wherein all the symbols have the same meanings as defined hereinbefore), i.e. the amide derivative of formula (I-2d) 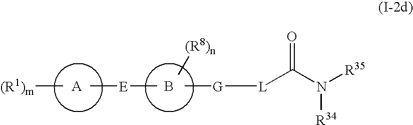
   (wherein all the symbols have the same meanings as defined hereinbefore), may be prepared by the following methods.
(2-d-1) Among the compounds of formula (I-2d), the compound that any of R¹, A, B, R⁸ or G does not represent the group which contains amino, thiol nor carboxyl group, i.e. the compound of formula (I-2d-1) 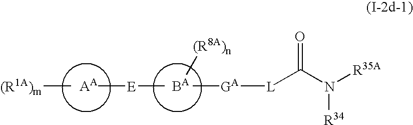
   (wherein all the symbols have the same meanings as defined hereinbefore) may be prepared by amidation of the carboxylic acid derivative of formula (II) and the amine derivative of formula (IX)
   HNR³⁴R³⁵ (IX)
   (wherein all the symbols have the same meanings as defined hereinbefore).

The amidation may be carried out by the same procedure as described in (1-a).
(2-d-2) Among the compounds of formula (I-2d), the compound that one or more of R¹, A, B, R⁸ and G represent(s) the group which contains amino, thiol or carboxyl, i.e. the compound of formula (I-2d-2) 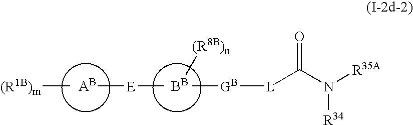
   (wherein all the symbols have the same meanings as defined hereinbefore), may be prepared by the removal of the protecting group of amino, thio or carboxyl of the compound that one or more of R^{1A}, A^{A}, B^{A}, R^{8A} and G^{A} represent(s) the group which contains protected amino, protected thiol or protected carboxyl among the compounds of formula (I-2a-1), i.e. the compound of formula (I-2d-1-1) 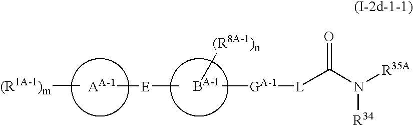
   (wherein all the symbols have the same meanings as defined hereinbefore).

The removal reaction may be carried out by the method of (1-b-1).

The compounds of the formula (II), (III), (IV), (V), (VI), (VII) and (VIII) have been known per se or may be prepared by known methods easily. The known methods are exemplified in Examples.

In each reaction in the present specification, an obtained product may be purified by conventional techniques. For example, purification may be carried out by distillation at atmospheric or reduced pressure, by high performance liquid chromatography, by thin layer chromatography or by column chromatography using silica gel or magnesium silicate, by washing or by recrystallization. Purification may be carried out after each reaction, or after a series of reactions.

The other starting materials and each test compound in the present invention have been known per se or may be prepared by known methods.

Pharmacological Activities of the Compound of the Present Invention:

According to following experiments, it is confirmed that the compound of the present invention has an inhibitory activity on IL-6 production.
(1) The Measurement of an Inhibitory Activity on IL-6 Production from A549 Cells
   Method:

1.5×10⁴ of A549 cells (human lung epithelial cell line) were suspended in 100 μL of Dulbecco's Modified Eagle Medium (DMEM) containing 0.5% fetal bovine serum (abbreviated as FBS) and incubated for a day and night in 96 well-microplate. 20 μL of the test compound dissolved in dimethylsulfoxide (DMSO) at various concentrations and 80 μl of tumor necrosis factor-α (TNF-α (Genzyme Co., cat. No. TNF-H)) dissolved in serum-free DMEM at the concentration of 12.5 ng/mL were added thereto. After the incubation for 24 hours, the supernatant (100 μL) was collected to measure the amount of IL-6 being produced using enzyme linked immuno solvent assay (ELISA) (R&D Systems Co., cat. No. D6050). Then the inhibitory activity of the test compound was calculated and the 50% inhibitory concentration (IC₅₀) was determined. For example, the IC₅₀ value of the compound of Example 2 was 0.18 μM.
(2) The Measurement of an Inhibitory Activity on IL-6 Production from Human Synovial Cells
   Method:

3.0×10³ of synovial cells from rheumatoid arthritis patients were suspended in 200 μL of DMEM containing 10% FBS and incubated for a day and night in 96 well-microplate, followed by the incubation for 5 hours in serum-free DMEM. 20 μl of the test compound dissolved in DMSO at various concentrations and 80 μL of interleukin-1β (Genzyme Co., cat. No. 80-3688-01)) dissolved in DMEM containing 2.5% fetal bovine serum at the concentration of 5 ng/mL were added thereto. After the incubation for 24 hours, the supernatant (100 μL) was collected to measure the amount of IL-6 being produced using ELISA (R&D Systems Co., cat No. D6050). Then the inhibitory activity of the test compound was calculated and the 50% inhibitory concentration (IC₅₀) was determined.
(3) The Effect on Collagen-Induced Arthritis Model in Rats
   Method:

Eight weeks old female DA rats (SLC) were used. During the experimental period, they were housed in animal room artificially kept the room temperature of 24±2° C., humidity of 55±5%, and 12 hours interval of light and dark cycle. They had free access to a standard solid pellet chow (CE-2, Japan CLEA) and drinking tap water, and were used for the experiment after a week acclimation. The collagen-induced arthritis was performed by the following method. After the mixing of bovine type II collagen (0.3% collagen solution, KOKEN #K-41, lot.11214, abbreviated as CII), incomplete Freund's adjuvant (DIDCO #0639-60, abbreviated as IFA) and saline with the ratio of 1:2:1, the mixture was then ultra-sonicated to form emulsion for 20 seconds×3 times at 1 interval. The intradermal injection of 0.1 mL of the emulsion (0.75 mg of CII/mL) was performed on each four different portions of the back on day 0. A week after the first immunization, arthritis was elicited by the intradermal injection of 0.15 mL of the emulsion at the basal portion of the tail. The test compound was suspended in 0.5% carboxymethylcellulose solution, and was administered orally in the morning and evening twice a day from day 0 to day 28. According to the method of Osterman T. et al. (Inflamm. Res., 44, 258-263, 1995), the severity of arthritis was judged and scored. The hind paw volume of each animal was also measured with the plethysmometer (UNICOM, TK-101CMP).
(4) The Measurement of an Inhibitory Activity on Proliferation of Various Tumor Cells (In Vitro)
   Method:

Various tumor cells were seeded in 96 well-microplate with cell density of 5,000 cells/well. After incubation for about 20 hours, the test compound was added thereto. After incubation for 24 hours, deoxybromouridine (BrDU) was added into the well for three hours. Then the amount of BrDU taken into the cells was measured by using ELISA kit (BOEHRINGER MANNHEIM, cat. No. 1-647-229), to calculate inhibitory activity of the compound for the BrDU uptake in the present invention and to determine 50% inhibitory concentration (IC₅₀) by concentration-inhibition curve.

Various tumor cells used in this experiment were obtained by following institutions:
A549: human lung cancer (adenocarcinoma); American Type Culture Colllection (ATCC),
Colon26: mouse rectal carcinoma; Tohoku University, Cell Resource Center for Biomedical Research,
A375: human melanoma cells; American Type Culture Collection (ATCC),
PANC-1: human pancreatic epitheloid carcinoma; American Type Culture Collection (ATCC),
KATOIII: human stomach carcinoma; American Type Culture Collection (ATCC),
HepG2: human hepatoma (hepato cellular); American Type Culture Collection (ATCC),
LU99: human giant cell carcinoma of lung; The Japan Health Sciences Foundation, Health Science Research Resources Bank,
LU65: human lung small cell carcinoma; The Japan Health Sciences Foundation, Health Science Research Resources Bank,
PC14: human lung cancer; RIKEN.
   Toxicity:

The toxicity of the compound of formula (I) of the present invention, the non-toxic salt thereof or the prodrug thereof is very low and therefore the compound may be considered safe for pharmaceutical use.

### INDUSTRIAL APPLICABILITY

Application to Pharmaceuticals

The compound of the present invention possess an inhibitory activity of IL-6 production in animal, especially human, so it is useful for the prevention and/or treatment of, for example, various inflammatory diseases, sepsis, multiple myeloma, plasma cell leukemia, osteoporosis, cachexia, psoriasis, nephritis, renal cell carcinoma, Kaposi's sarcoma, rheumatoid arthritis, hypergammaglobulinemia, Castleman's disease, atrial myxoma, diabetes mellitus, autoimmune diseases, hepatitis, colitis, graft versus host disease, infectious diseases and endometriosis.

The compound of the present invention is also useful for the prevention and/or treatment of solid cancer (e.g. brain tumor, head and neck tumor, thyroid cancer, esophageal cancer, gastric cancer, colorectal cancer (colon cancer and rectal cancer), liver cancer, gallbladder cancer, bileduct cancer, pancreatic cancer, lung cancer, breast cancer, cervical cancer, endometrial carcinoma, ovarian cancer, carcinoma of the prostate, orchioncus, bladder carcinoma, renal pelvic tumor, ureteral tumor, adrenal tumor, neuroma, glioma, osteoncus, rhabdomyosarcoma, osteosarcoma, soft tissue tumors, oxyphilic granuloma, malignant melanoma, cutaneous carcinoma, glioblastoma, Wilms tumor, etc.).

The compound of formula (I) of the present invention and a non-toxic salt thereof may be administered in combination with other medicaments for the purpose of
1) complement and/or enhancement of the prevention and/or treatment effect of the compound,
2) improvement of the pharmacokinetics and/or the absorption of the compound, lowering of dose, and/or
3) alleviation of a side effect of the compound.

The compound of formula (I) may be administered in combination with other medicaments as a composition in one drug product comprising these components, or may be separately administered. In the case of the separated administration, they may be administered simultaneously or with lapse of time. While administration with lapse of time, the compound of formula (I) may be precedently administered, followed by administration of the other medicaments. Alternatively, the other medicaments may be precedently administered, followed by administration of the compound of formula (I). Routes of administration may be either the same or different to each other.

The above combination drug takes effect on whichever disease preventing and/or treatment effect of the compound of formula (I) is complemented and/or enhanced.

For the purpose above described, the compound of formula (I), the non-toxic salt thereof or the prodrug thereof or combination of theirs and other medicaments may be normally administered systemically or locally, usually by oral or parenteral administration.

The doses to be administered are determined depending upon, for example, age, body weight, symptom, the desired therapeutic effect, the route of administration, and the duration of the treatment. In the human adult, the doses per person are generally from 1 mg to 1000 mg, by oral administration, up to several times per day, and from 0.1 mg to 100 mg, by parenteral administration (preferably intravenous administration), up to several times per day, or continuous administration from 1 to 24 hours per days from vein.

As mentioned above, the doses to be used depend upon various conditions. Therefore, there are cases in which doses lower than or great than the ranges specified above may be used.

The compound of the present invention or a combination of the compound of the present invention and other medicaments may be administered in the form of, for example, solid forms for oral administration, liquid forms for oral administration, injections, liniments or suppositories for parenteral administration.

Solid forms for oral administration include compressed tablets, pills, capsules, dispersible powders, and granules. Capsules include hard capsules and soft capsules.

In such solid forms, one or more of the active compound(s) may be admixed with vehicles (such as lactose, mannitol, glucose, microcrystalline cellulose or starch), binders (such as hydroxypropyl cellulose, polyvinylpyrrolidone or magnesium metasilicate aluminate), disintegrants (such as cellulose calcium glycolate), lubricants (such as magnesium stearate), stabilizing agents, and solution adjuvants (such as glutamic acid or aspartic acid) and prepared according to methods well known in normal pharmaceutical practice. The solid forms may, if desired, be coated with coating agents (such as sugar, gelatin, hydroxypropyl cellulose or hydroxypropylmethyl cellulose phthalate), or be coated with two or more films. And further, coating may include containment within capsules of absorbable materials such as gelatin.

Liquid forms for oral administration include pharmaceutically acceptable solutions, suspensions and emulsions, syrups, elixirs, etc. In such forms, one or more of the active compound(s) may be dissolved, suspended or emulsified into diluent(s) commonly used in the art (such as purified water, ethanol or a mixture thereof). Furthermore, such liquid forms may also comprise some additives, such as wetting agents, suspending agents, emulsifying agents, sweeting agents, flavoring agents, aroma, preservative, buffering agent, etc.

Injections for parenteral administration include sterile aqueous, suspensions, emulsions and solid forms which are dissolved or suspended into solvent(s) for injection immediately before use. In injections, one or more of the active compound(s) may be dissolved, suspended or emulsified into solvent(s). The solvents may include distilled water for injection, physiological salt solution, vegetable oil, propylene glycol, polyethylene glycol, alcohol, e.g. ethanol, or a mixture thereof. Injections may comprise some additives, such as stabilizing agents, solution adjuvants (such as glutamic acid, aspartic acid or POLYSORBATE80 (registered trade mark)), suspending agents, emulsifying agents, soothing agents, buffering agents, preservative. They may be sterilized at a final step, or may be prepared and compensated according to sterile methods. They may also be manufactured in the form of sterile solid forms such as freeze-dried products, which may be dissolved in sterile water or some other sterile diluent(s) for injection immediately before use.

Other forms for parenteral administration include liquids for external use, ointments and endermic liniments, inhalations, sprays, suppositories and pessaries for vaginal administration which comprise one or more of the active compound(s) and may be prepared by methods known per se.

Sprays may comprise additional substances other than diluents, such as stabilizing agents (such as sodium sulfate), isotonic buffers (such as sodium chloride, sodium citrate or citric acid). For preparation of such sprays, for example, the method described in the U.S. Pat. No. 2,868,691 or 3,095,355 may be used.

### BEST MODE FOR CARRYING OUT THE INVENTION

The following Reference Examples and Examples are intended to illustrate the present invention, but do not limit them.

In chromatographic separations and TLC, the solvents in parenthesis show the eluting and developing solvents and the ratios of the solvents used are by volume.

The solvents in parenthesis in NMR show the solvents used for measurement.

TBS in formulae represents t-butyldimethylsilyl.

### Reference Example 1

6-[(4-phenylbenzoyl)amino]hexanoic acid

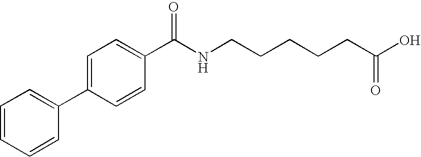

4-Phenylbenzoylchloride (1.73 g) in tetrahydrofuran (8 ml) was added to the solution of 6-aminohexanoic acid (1.05 g) in 2N aqueous solution of sodium hydroxide (8 ml) at 0° C., then the mixture was stirred for 1 hour at 0° C. To the mixture of reaction, 2N hydrochloric acid (8.5 ml) was added and extracted with the mixed solvent of ethyl acetate and tetrahydrofuran. The organic layer was washed with brine, dried over anhydrous magnesium sulfate and concentrated to give the total compound (2.35 g) having the following physical data.
TLC: Rf 0.40 (chloroform:methanol=9:1);
NMR (CDCl₃+DMSO-d₆): δ 7.90-7.82 (m, 2H), 7.68-7.58 (m, 4H), 7.50-7.34 (m, 3H), 6.53 (t, J=5.7 Hz, 1H), 3.49 (q, J=5.7 Hz, 2H), 2.33 (t, J=7.2 Hz, 2H), 1.75-1.60 (m, 4H), 1.54-1.40 (m, 2H).

### Example 1

(N-(1-methyl-1-methoxyethoxy)-6[(4-phenylbenzoyl)amino]hexanamide

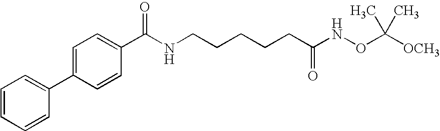

Under atmosphere of argon, to a solution of the compound prepared in Reference Example 1 (2.24 g) in N,N-dimethylformamide (42 ml), 1-hydroxybenzotriazole hydrate (1.65 g), triethylamine (2.91 g), 1-ethyl-3-[3-(dimethylamino)propyl]carbidiimide hydrochloride (2.07 g) and N-(1-methyl-1-methoxyethoxy)amine (1.14 g) were added sequentially and the mixture was stirred for 4 hours at room temperature.

The reaction mixture was poured into water and extracted with ethyl acetate. The organic layer was washed with 1N hydrochloric acid, water, saturated aqueous solution of sodium hydrogen carbonate and brine sequentially, dried over anhydrous magnesium sulfate, concentrated under reduced pressure. The residue was purified by column chromatography on silica gel (ethyl acetate:n-hexane:triethylamine=80:20:1) to give the title compound (1.79 g) having the following physical data.
TLC: Rf 0.36 (ethyl acetate:triethylamine=20:1);
NMR (DMSO-d₆): δ 10.25 (s, 1H), 8.48 (t, J=6.0 Hz, 1H), 7.93 (d, J=8.1 Hz, 2H), 7.75 (d, J=8.1 Hz, 2H), 7.73 (d, J=7.2 Hz, 2H), 7.49 (t, J=7.2 Hz, 2H), 7.45-7.35 (m, 1H), 3.26 (q, J=6.0 Hz, 2H), 3.20 (s, 3H), 2.03 (t, J=7.2 Hz, 2H), 1.62-1.46 (m, 4H), 1.36-1.24 (m, 2H), 1.25 (s, 6H).

### Example 2

N-hydroxy-6-[(4-phenylbenzoyl)amino]hexanamide

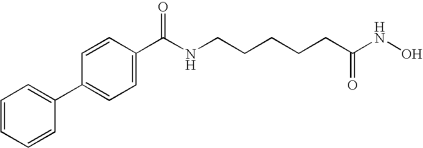

To a solution of the compound prepared in Example 1 (1.78 g) in methanol (4.5 ml), 2N hydrochloric acid (4.5 ml) was added and the mixture was stirred at room temperature. The reaction mixture was concentrated to give the compound of the present invention (1.24 g) having the following physical data.
TLC: Rf 0.18 (chloroform:methanol:triethylamine=10:1:1);
NMR (DMSO-d₆): δ 10.33 (s, 1H), 8.65 (bs, 1H), 8.49 (t, J=6.0 Hz, 1H), 7.93 (d, J=8.1 Hz, 2H), 7.75 (d, J=8.1 Hz, 2H), 7.72 (d, J=7.5 Hz, 2H), 7.49 (t, J=7.5 Hz, 2H), 7.45-7.36 (m, 1H), 3.26 (q, J=6.0 Hz, 2H), 1.96 (t, J=7.2 Hz, 2H), 1.60-1.46 (m, 4H), 1.36-1.23 (m, 2H).

### Example 2(1) to Example 2(145)

By the same procedure as described in Example 2 using corresponding compounds, the compounds of the present invention having the following physical data were obtained. The corresponding compounds may be prepared by the same procedure as described in Example 1 using corresponding carboxylic acid and hydroxyamine derivatives.

### Example 2(1)

N-hydroxy-3-[(4-phenylbenzoyl)amino]propanamide 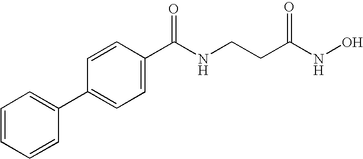
TLC: Rf 0.10 (chloroform:methanol=9:1);
NMR (DMSO-d₆): δ 10.41 (bs, 1H), 8.74 (bs, 1H), 8.58 (t, J=7.2 Hz, 1H), 7.93 (d, J=8.1 Hz, 2H), 7.76 (d, J=8.1 Hz, 2H), 7.73 (d, J=7.5 Hz, 2H), 7.49 (t, J=7.5 HZ, 2H), 7.44-7.36 (m, 1H), 3.47 (q, J=7.2 Hz, 2H), 2.28 (t, J=7.2 Hz, 2H).

### Example 2(2)

N-hydroxy-4-[(4-phenylbenzoyl)amino]butanamide 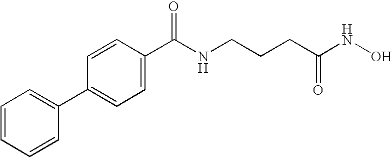
TLC: Rf 0.10 (chloroform:methanol=9:1);
NMR (DMSO-d₆): δ 10.40 (bs, 1H), 8.71 (bs, 1H), 8.55 (t, J=7.2 Hz, 1H), 7.94 (d, J=8.4 Hz, 2H), 7.76 (d, J=8.4 Hz, 2H), 7.73 (d, J=7.2 Hz, 2H), 7.50 (t, J=7.2 Hz, 2H), 7.44-7.36 (m, 1H), 3.28 (q, J=7.2 Hz, 2H), 2.04 (t, J=7.2 Hz, 2H), 1.77 (quint, J=7.2 Hz, 2H).

### Example 2(3)

N-hydroxy-5-[(4-phenylbenzoyl)amino]pentanamide 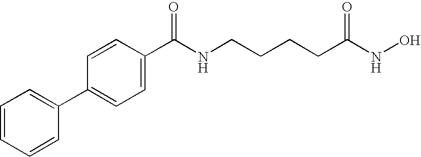
TLC: Rf 0.13 (chloroform:methanol:triethylamine=10:1:1);
NMR (DMSO-d₆): δ 10.35 (s, 1H), 8.66 (s, 1H), 8.51 (t, J=5.7 Hz, 1H), 7.93 (d, J=8.1 Hz, 2H), 7.75 (d, J=8.1 Hz, 2H), 7.72 (d, J=7.2 Hz, 2H), 7.49 (t, J=7.2 Hz, 2H), 7.45-7.36 (m, 1H), 3.29 (q, J=5.7 Hz, 2H), 1.99 (t, J=6.9 Hz, 2H), 1.60-1.45 (m, 4H).

### Example 2(4)

N-hydroxy-7-[(4-phenylbenzoyl)amino]heptanamide 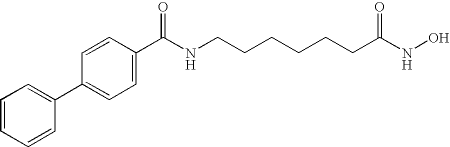
TLC: Rf 0.23 (chloroform:methanol:triethylamine=10:1:1);
NMR (DMSO-d₆): δ 10.33 (s, 1H), 8.66 (bs, 1H), 8.48 (t, J=5.7 Hz, 1H), 7.93 (d, J=8.4 Hz, 2H), 7.75 (d, J=8.4 Hz, 2H), 7.72 (d, J=7.2 Hz, 2H), 7.49 (t, J=7.2 Hz, 2H), 7.45-7.36 (m, 1H), 3.26 (q, J=5.7 Hz, 2H), 1.95 (t, J=7.5 Hz, 2H), 1.60-1.43 (m, 4H), 1.38-1.20 (m, 4H).

### Example 2(5)

N-hydroxy-3-{[4-(4-chlorophenyl)benzoyl]amino}propanamide 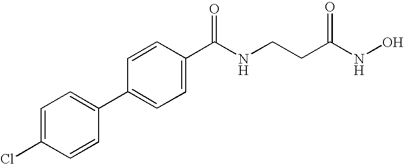
TLC: Rf 0.13 (chloroform:methanol:triethylamine=10:1:1);
NMR (DMSO-d₆): δ 10.45 (s, 1H), 8.74 (s, 1H), 8.60 (t, J=6.0 Hz, 1H), 7.93 (d, J=8.7 Hz, 2H), 7.77 (d, J=8.7 Hz, 2H), 7.76 (d, J=8.7 Hz, 2H), 7.54 (d, J=8.7 Hz, 2H), 3.48 (q, J=6.0 Hz, 2 H), 2.28 (t, J=6.0 Hz, 2H).

### Example 2(6)

N-hydroxy-5-{[4-(4-chlorophenyl)benzoyl]amino}pentanamide 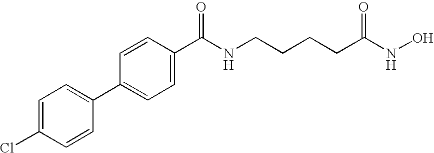
TLC: Rf 0.16 (chloroform:methanol:triethylamine=10:1:1);
NMR (DMSO-d₆): δ 10.35 (s, 1H), 8.66 (s, 1H), 8.52 (t, J=5.7 Hz, 1H), 7.94 (d, J=8.7 Hz, 2H), 7.76 (d, J=8.7 Hz, 4H), 7.54 (d, J=8.7 Hz, 2H), 3.26 (q, J=5.7 Hz, 2H), 1.99 (t, J=6.9 Hz, 2H), 1.60-1.42 (m, 4H).

### Example 2(7)

N-hydroxy-6-{[4-(4-chlorophenyl)benzoyl]amino}hexanamide 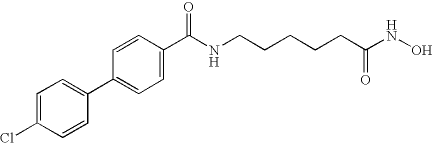
TLC: Rf 0.20 (chloroform:methanol:triethylamine=10:1:1);
NMR (DMSO-d₆): δ 10.33 (s, 1H), 8.65 (s, 1H), 8.50 (t, J=6.0 Hz, 1H), 7.93 (d, J=8.7 Hz, 2H), 7.76 (d, J=8.7 Hz, 4H), 7.54 (d, J=8.7 Hz, 2H), 3.26 (q, J=6.0 Hz, 2H), 1.95 (t, J=7.5 Hz, 2H), 1.60-1.45 (m, 4H), 1.36-1.20 (m, 2H).

### Example 2(8)

N-hydroxy-7-{[4-(4-chlorophenyl)benzoyl]amino}heptanamide 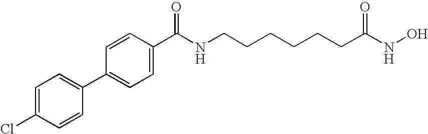
TLC: Rf 0.20 (chloroform:methanol:triethylamine=10:1:1);
NMR (DMSO-d₆): δ 10.33 (s, 1H), 8.65 (s, 1H), 8.49 (t, J=6.0 Hz, 1H), 7.93 (d, J=8.4 Hz, 2H), 7.76 (d, J=8.4 Hz, 4H), 7.54 (d, J=8.4 Hz, 2H); 3.26 (q, J=6.0 Hz, 2H), 1.94 (t, J=7.2 Hz, 2 H), 1.60-1.40 (m, 4H), 1.40-1.20 (m, 4H).

### Example 2(9)

N-hydroxy-3-[(4-cyclohexylbenzoyl)amino]propanamide 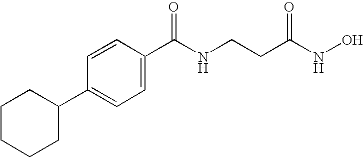
TLC: Rf 0.17 (chloroform:methanol:triethylamine=10:1:1);
NMR (DMSO-d₆): δ 10.43 (s, 1H), 8.72 (s, 1H), 8.42 (t, J=6.0 Hz, 1H), 7.74 (d, J=8.4 Hz, 2H), 7.29 (d, J=8.4 Hz, 2H), 3.43 (q, J=6.0 Hz, 2H), 2.62-2.40 (m, 1H), 2.25 (t, J=7.2 Hz, 2H) 1.86-1.64 (m, 5H), 1.50-1.15 (m, 5H).

### Example 2(10)

N-hydroxy-5-[(4-cyclohexylbenzoyl)amino]pentanamide 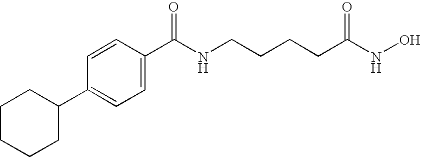
TLC: Rf 0.18 (chloroform:methanol:triethylamine=10:1:1);
NMR (DMSO-d₆): δ 10.36 (bs, 1H), 8.37 (t, J=5.7 Hz, 1H), 7.75 (d, J=8.4 Hz, 2H), 7.28 (d, J=8.4 Hz, 2H), 3.22 (q, J=5.7 Hz, 2H), 2.60-2.45 (m, 1H), 1.97 (t, J=6.6 Hz, 2H), 1.84-1.64 (m, 5H), 1.58-1.16 (m, 9H).

### Example 2(11)

N-hydroxy-6-[(4-cyclohexylbenzoyl)amino]hexanamide 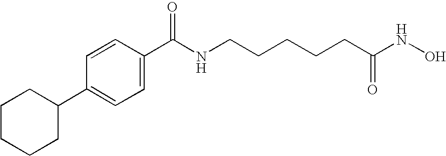
TLC: Rf 0.21 (chloroform:methanol:triethylamine=10:1:1);
NMR (DMSO-d₆): δ 10.33 (bs, 1H), 8.33 (t, J=6.0 Hz, 1H), 7.75 (d, J=8.4 Hz, 2H), 7.28 (d, J=8.4 Hz, 2H), 3.22 (q, J=6.0 Hz, 2H), 2.60-2.40 (m, 1H), 1.94 (t, J=7.2 Hz, 2H), 1.85-1.64 (m, 5H), 1.57-1.15 (m, 11H).

### Example 2(12)

N-hydroxy-7-[(4-cyclohexylbenzoyl)amino]heptanamide 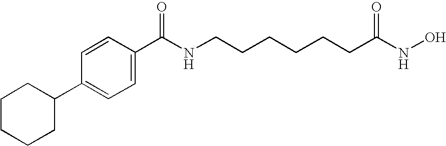
TLC: Rf 0.25 (chloroform:methanol:triethylamine=10:1:1);
NMR (DMSO-d₆): δ 10.32 (s, 1H), 8.32 (t, J=6.3 Hz, 1H), 7.74 (d, J=8.4 Hz, 2H), 7.28 (d, J=8.4 Hz, 2H), 3.20 (q, J=6.3 Hz, 2H), 2.60-2.40 (m, 1H), 1.93 (t, J=7.2 Hz, 2H), 1.84-1.63 (m, 5H), 1.55-1.15 (m, 13H).

### Example 2(13)

N-hydroxy-3-(benzoylamino)propanamide 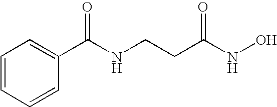
TLC: Rf 0.13 (chloroform:methanol:triethylamine=10:1:1);
NMR (DMSO-d₆): δ 10.48 (bs, 1H), 8.56 (t, J=6.0 Hz, 1H), 7.88-7.78 (m, 2H), 7.57-7.40 (m, 3H), 3.46 (q, J=6.0 Hz, 2H), 2.28 (t, J=6.0 Hz, 2H).

### Example 2(14)

N-hydroxy-4-(benzoylamino)butanamide 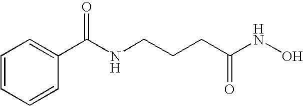
TLC: Rf 0.13 (chloroform:methanol:triethylamine=10:1:1);
NMR (DMSO-d₆): δ 10.42 (bs, 1H), 8.51 (t, J=6.0 Hz, 1H), 7.90-7.80 (m, 2H), 7.55-7.40 (m, 3H), 3.31 (q, J=6.0 Hz, 2H), 2.03 (t, J=7.5 Hz, 2H), 1.84-1.69 (m, 2H).

### Example 2(15)

N-hydroxy-5-(benzoylamino)pentanamide 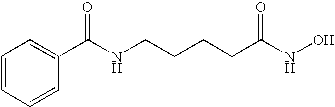
TLC: Rf 0.13 (chloroform:methanol:triethylamine=10:1:1);
NMR (DMSO-d₆): δ 10.35 (s, 1H), 8.46 (t, J=5.7 Hz, 1H), 7.87-7.79 (m, 2H), 7.56-7.40 (m, 3H), 3.24 (q, J=5.7 Hz, 2H), 1.98 (t, J=6.9 Hz, 2H), 1.60-1.42 (m, 4H).

### Example 2(16)

N-hydroxy-6-(benzoylamino)hexanamide 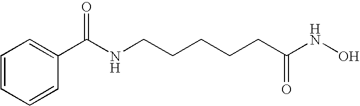
TLC: Rf 0.13 (chloroform:methanol:triethylamine=10:1:1);
NMR (DMSO-d₆): δ 10.33 (s, 1H), 8.43 (t, J=6.0 Hz, 1H), 7.87-7.78 (m, 2H), 7.54-7.40 (m, 3H), 3.24 (q, J=6.0 Hz, 2H), 1.95 (t, J=7.2 Hz, 2H), 1.60-1.45 (m, 4H), 1.36-1.20 (m, 2H).

### Example 2(17)

N-hydroxy-7-(benzoylamino)heptanamide 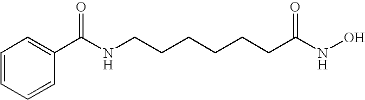
TLC: Rf 0.13 (chloroform:methanol:triethylamine=10:1:1);
NMR (DMSO-d₆): δ 10.33 (s, 1H), 8.42 (t, J=6.0 Hz, 1H), 7.88-7.80 (m, 2H), 7.56-7.40 (m, 3H), 3.24 (q, J=6.0 Hz, 2H), 1.94 (t, J=7.8 Hz, 2H), 1.58-1.42 (m, 4H), 1.38-1.20 (m, 4H).

### Example 2(18)

N-hydroxy-6-{[4-(4-cyanophenyl)benzoyl]amino}hexanamide 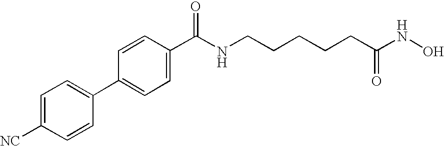
TLC: Rf 0.34 (chloroform:methanol:acetic acid=90:10:1);
NMR (DMSO-d₆): δ 10.32 (s, 1H), 8.70-8.40 (m, 2H), 8.02-7.90 (m, 6H), 7.84 (d, J=8.4 Hz, 2H), 3.30-3.20 (m, 2H, overlap with H₂O in DMSO), 1.94 (t, J=7.2 Hz, 2H), 1.60-1.45 (m, 4H), 1.40-1.20 (m, 2H).

### Example 2(19)

N-hydroxy-6-{[4-(4-propylphenyl)benzoyl]amino}hexanamide 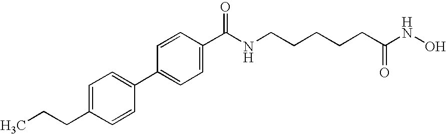
TLC: Rf 0.38 (chloroform:methanol:acetic acid=90:10:1);
NMR (DMSO-d₆): δ 10.32 (s, 1H), 8.64 (s, 1H), 8.46 (t, J=5.4 Hz, 1H), 7.90 (d, J=8.6 Hz, 2H), 7.72 (d, J=8.6 Hz, 2H), 7.63 (d, J=8.3 Hz, 2H), 7.29 (d, J=8.3 Hz, 2H), 3.30-3.20 (m, 2H, overlap with H₂O in DMSO), 2.59 (t, J=7.5 Hz, 2H), 1.94 (t, J=7.2 Hz, 2H), 1.70-1.40 (m, 6H), 1.40-1.20 (m, 2H), 0.91 (t, J=7.2 Hz, 3H).

### Example 2(20)

N-hydroxy-6-[(4-phenoxybenzoyl)amino]hexanamide 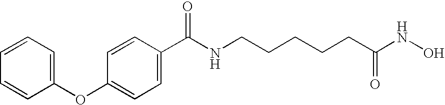
TLC: Rf 0.14 (ethyl acetate);
NMR (DMSO-d₆): δ 10:31 (s, 1H), 8.64 (s, 1H), 8.36 (t-like, 1H), 7.85 (d, J=8.4 Hz, 2H), 7.45-7.39 (m, 2H), 7.21-7.16 (m, 1H), 7.08-7.05 (m, 2H), 7.00 (d, J=8.4 Hz, 2H), 3.21 (q, J=6.6 Hz, 2H), 1.93 (t, J=7.2 Hz, 2H), 1.55-1.45 (m, 4H), 1.30-1.22 (m, 2H).

### Example 2(21)

N-hydroxy-6-{[4-(4-methoxyphenoxy)benzoyl]amino}hexanamide 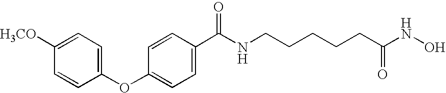
TLC: Rf 0.15 (ethyl acetate);
NMR: (DMSO-d₆): δ 10.31 (s, 1H), 8.63 (s, 1H), 8.32 (t-like, 1H), 7.81 (d, J=9.0 Hz, 2H), 7.04 (d, J=9.0 Hz, 2H), 6.98 (d, J=9.0 Hz, 2H), 6.91 (d, J=9.0 Hz, 2H), 3.75 (s, 3H), 3.20 (q, J=6.6 Hz, 2H), 1.93 (t, J=7.2 Hz, 2H), 1.54-1.43 (m, 4H), 1.29-1.22 (m, 2H).

### Example 2(22)

N-hydroxy-6-{[4-(3-phenoxyprop-1-ynyl)benzoyl]amino}hexanamide 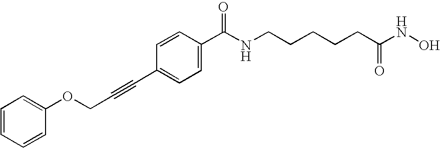
TLC: Rf 0.23 (ethyl acetate);
NMR (DMSO-d₆): δ 10.30 (s, 1H), 8.63 (s, 1H), 8.50 (t, J=5.1 Hz, 1H), 7.81 (d, J=8.4 Hz, 2H), 7.50 (d, J=8.4 Hz, 2H), 7.34-7.29 (m, 2H), 7.04-6.95 (m, 3H), 5.05 (s, 2H), 3.24-3.17 (m, 2H), 1.90 (t, J=7.2 Hz, 2H), 1.53-1.44 (m, 4H), 1.30-1.22 (m, 2H).

### Example 2(23)

N-hydroxy-6-{[4-(3-methoxyprop-1-ynyl)benzoyl]amino}hexanamide 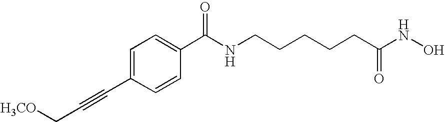
TLC: Rf 0.23 (ethyl acetate);
NMR (DMSO-d₆): δ 10.30 (s, 1H), 8.63 (s, 1H), 8.49 (t, J=5.1 Hz, 1H), 7.82 (d, J=8.1 Hz, 2H), 7.52 (d, J=8.1 Hz, 2H), 4.33 (s, 2H), 3.32(s, 3H), 3.24-3.19 (m, 1H), 1.92 (t, J=7.5 Hz, 2H), 1.54-1.44 (m, 4H), 1.30-1.21 (m, 2H).

### Example 2(24)

N-hydroxy-6-[methyl(4-phenylbenzoyl)amino]hexanamide 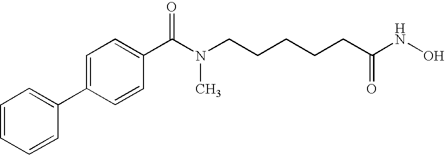
TLC: Rf 0.13 (ethyl acetate);
NMR (DMSO-d₆): δ 10.31 (brs, 1H), 7.72-7.68 (m, 4H), 7.50-7.36 (m, 5H), 3.42 (br, 1H), 3.20 (br, 1H), 2.92 (brs, 3H), 1.98-1.86 (m, 2H), 1.55-1.25 (m, 5H), 1.05 (br, 1H).

### Example 2(25)

N-hydroxy-6-{[5-(4-methoxyphenyl)thiophen-2-ylcarbonyl]amino}hexanamide 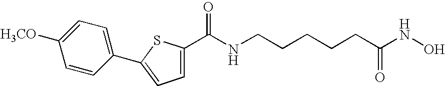
TLC: Rf 0.20 (chloroform:methanol=9:1);
NMR (DMSO-d₆): δ 10:31 (s, 1H), 8.64 (brs, 1H), 8.41 (t, J=5.4 Hz, 1H), 7.67 (d, J=3.9 Hz, 1H), 7.61 (d, J=8.7 Hz, 2H), 7.37 (d, J=3.9 Hz, 1H), 6.98 (d, J=8.7 Hz, 2H), 3.77 (s, 3H), 3.22-3.16 (m, 2H), 1.93 (t, J=6.9 Hz, 2H), 1.59-1.40 (m, 4H), 1.31-1.20 (m, 2H).

### Example 2(26)

N-hydroxy-6-{[4-(3-methoxyphenoxy)benzoyl]amino}hexanamide 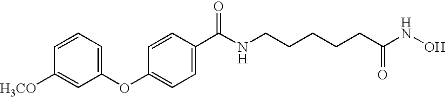
TLC: Rf 0.24 (chloroform:methanol=9:1);
NMR (DMSO-d₆): δ 10.30 (s, 1H), 8.63 (s, 1H), 8.36 (t, J=5.4 Hz, 1H), 7.84 (d, J=9.0 Hz, 2H), 7.30 (t, J=8.1 Hz, 1H), 7.02 (d, J=9.0 Hz, 2H), 6.78-6.74 (m, 1H), 6.64-6.58 (m, 2H), 3.72 (s, 3H), 3.24-3.17 (m, 1H), 1.93 (t, J=7.2 Hz, 2H), 1.58-1.41 (m, 4H), 1.31-1.22 (m, 2H).

### Example 2(27)

(6S)-N-hydroxy-7-ethoxymethoxy-6-[(4-phenylbenzoyl)amino]heptanamide 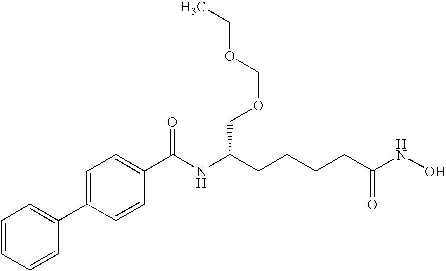
TLC: Rf 0.35 (chloroform:methanol:acetic acid=9:1:0.1);
NMR (DMSO-d₆): δ 10:30 (s, 1H), 8.63 (s, 1H), 8.20 (d, J=8.0 Hz, 1H), 7.94 (d, J=8.0 Hz, 2H), 7.80-7.65 (m, 4H), 7.60-7.30 (m, 3H), 4.61 (s, 2H), 4.20-4.00 (m, 1H), 3.60-3.40 (m, 4H), 1.93 (t, J=7.0 Hz, 2H), 1.75-1.20 (m, 6H), 1.10 (t, J=7.1 Hz, 3H).

### Example 2(28)

(6S)-N-hydroxy-7-hydroxy-6-[(4-phenylbenzoyl)amino]heptanamide 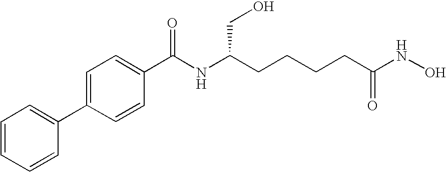
TLC: Rf 0.33 (chloroform:methanol:acetic acid=6:1:0.1);
NMR (DMSO-d₆): δ 10.31 (s, 1H), 8.63 (s, 1H), 8.05 (d, J=8.2 Hz, 1H), 7.95 (d, J=8.4 Hz, 2H), 7.80-7.65 (m, 4H), 7.60-7.30 (m, 3H), 4.68 (t, J=5.5 Hz, 1H), 4.05-3.83 (m, 1H), 3.55-3.20 (m, 2H), 1.93 (t, J=7.2 Hz, 2H), 1.80-1.10 (m, 6H).

### Example 2(29)

(6S, 2E)-N-hydroxy-7-ethoxymethoxy-2-methyl-6-[(4-phenylbenzoyl)amino]hept-2-enamide 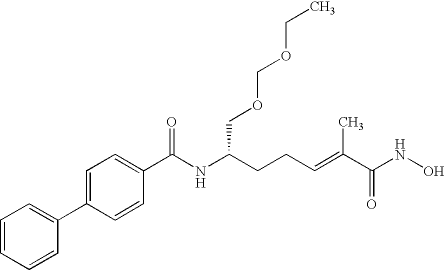
TLC: Rf 0.38 (chloroform:methanol:acetic acid=9:1:01);
NMR (DMSO-d₆): δ 10.65-10.30 (br, 1H), 8.80-8.50 (br, 1H), 8.26 (d, J=8.4 Hz, 1H), 7.95 (d, J=8.4 Hz, 2H), 7.85-7.65 (m, 4H), 7.60-7.30 (m, 3H), 6.30-6.10 (m, 1H), 4.61 (s, 2H), 4.22-4.05 (m, 1H), 3.60-3.40 (m, 4H), 2.25-2.05 (m, 2H), 1.80-1.50 (m, 5H), 1.10 (t, J=7.2 Hz, 3H).

### Example 2(30)

(6S)-N-hydroxy-7-ethoxymethoxy-2-methyl-6-[(4-phenylbenzoyl)amino]heptanamide 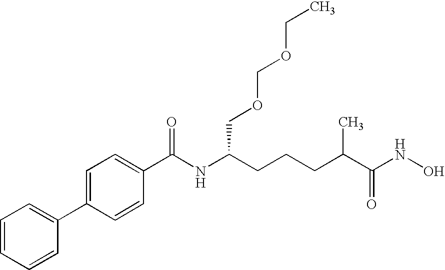
TLC: Rf 0.47 (chloroform:methanol:acetic acid=9:1:0.1);
NMR (DMSO-d₆): δ 10.34 (s, 1H), 8.65 (s, 1H), 8.20 (d, J=8.0 Hz, 1H), 7.94 (d, J=8.2 Hz, 2H), 7.80-7.65 (m, 4H), 7.60-7.30 (m, 3H), 4.60 (s, 2H), 4.20-4.00 (m,4H), 3.60-3.40 (m, 4H), 2.20-1.98 (m, 1H), 1.65-1.18 (m, 6H), 1.09 (t, J=7.2 Hz, 3H), 0.95 (d, J=7.0 Hz, 3H).

### Example 2(31)

N-hydroxy-5-[methyl(4-phenylbenzoyl)amino]pentanamide 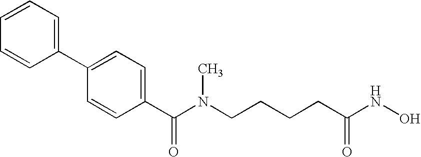
TLC: Rf 0.12 (ethyl acetate);
NMR (DMSO-d₆): δ 10.33 (brs, 1H), 8.66 (brs, 1H), 7.73-7.68 (m, 4H), 7.50-7.36 (m, 5H), 3.44 (br, 1H), 3.23 (br, 1H), 2.91 (brs, 3H), 2.01 (br, 1H), 1.86 (br, 1H), 1.54 (br, 3H), 1.30 (br, 1H).

### Example 2(32)

N-hydroxy-4-[(4-phenylbenzoyl)aminomethyl]benzamide 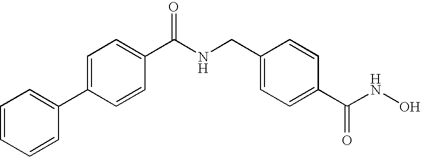
TLC: Rf 0.29 (ethyl acetate);
NMR (DMSO-d₆): δ 11.16 (s, 1H), 9.14 (t, J=6.0 Hz, 1H), 7.99 (d, J=8.4 Hz, 2H), 7.78 (d, J=8.4 Hz, 2H), 7.73-7.69 (m, 4H), 7.51-7.46 (m, 2H), 7.42-7.37 (m, 3H), 4.52 (d, J=6.0 Hz, 2H).

### Example 2(33)

N-hydroxy-3-[(1R, 3R)-3-[(4-phenylbenzoyl)amino]cyclohexyl]propanamide 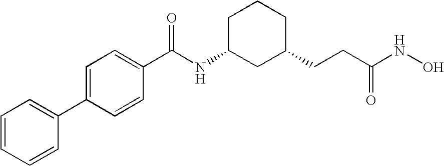
TLC: Rf 0.25 (chloroform:methanol=9:1);
NMR (DMSO-d₆): δ 10.34 (s, 1H), 8.66 (brs, 1H), 8.25 (d, J=8.1 Hz, 1H), 7.92 (d, J=8.4 Hz, 2H), 7.75-7.69 (m, 4H), 7.50-7.36 (m, 3H), 3.83-3.70 (m, 1H), 1.97 (t, J=7.8 Hz, 2H), 1.88-1.64 (m, 4H), 1.51-0.71 (m, 7H).

### Example 2(34)

(2E)-N-hydroxy-6-[(4-phenylbenzoyl)amino]hex-2-enamide 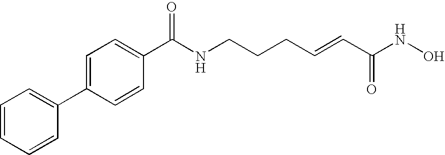
TLC: Rf 0.14 (chloroform:methanol=9:1);
NMR (DMSO-d₆): δ 10.52 (s, 1H), 8.80 (brs, 1H), 8.52 (t, J=5.7 Hz, 1H), 7.92 (d, J=8.4 Hz, 2H), 7.74 (d, J=8.4 Hz, 2H), 7.71 (d, J=7.5 Hz, 2H), 7.50-7.36 (m, 3H), 6.65 (dt, J=15.9 Hz, 6.6 Hz, 1H), 5.75 (d, J=15.9 Hz, 1H), 3.30-3.23 (m, 2H), 2.21-2.14 (m, 2H), 1.69-1.60 (m, 2H).

### Example 2(35)

(6R)-N-hydroxy-7-ethoxymethoxy-6-[(4-phenylbenzoyl)amino]heptanamide 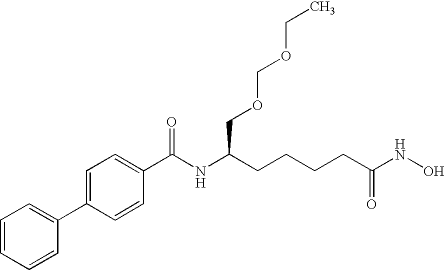
TLC: Rf 0.40 (chloroform:methanol:acetic acid=9:1:0.1);
NMR (DMSO-d₆): δ 10.40-10.20 (br, 1H), 9.20-8.25 (br, 1H), 8.21 (d, J=8.4 Hz, 1H), 7.94 (d, J=7.8 Hz, 2H), 7.85-7.60 (m, 4H), 7.60-7.30 (m, 3H), 4.61 (s, 2H), 4.20-4.00 (m, 1H), 3.70-3.10 (m, 4H, overlap with H₂O in DMSO), 1.93 (t, J=6.8 HZ, 2H), 1.70-1.40 (m, 4H), 1.40-1.20 (m, 2H), 1.10 (t, J=6.9 Hz, 3H).

### Example 2(36)

N-hydroxy-3-({2-[(4-phenylbenzoyl)amino]acetyl}amino)propanamide 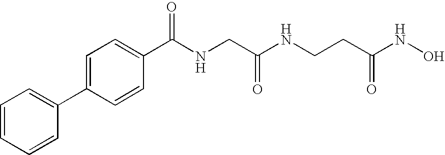
TLC: Rf 0.26 (chloroform:methanol:acetic acid=6:1:0.1);
NMR (DMSO-d₆): δ 10.42 (s, 1H), 8.77 (t, J=5.8 Hz, 1H), 8.10-7.85 (m, 3H), 7.85-7.65 (m, 4H), 7.60-7.30 (m, 3H), 3.84 (d, J=5.8 Hz, 2H), 3.60-3.20 (m, 2H, overlap with H₂O in DMSO), 2.14 (t, J=7.4 Hz, 2H).

### Example 2(37)

N-hydroxy-2-({3-[(4-phenylbenzoyl)amino]propanoyl}amino) acetamide 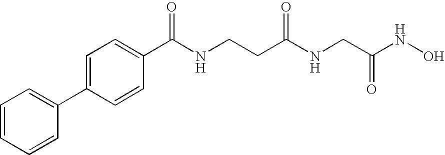
TLC: Rf 0.33 (chloroform:methanol:acetic acid=6:1:0.1);
NMR (DMSO-d₆): δ 10.52 (s, 1H), 8.90-8.70 (br, 1H), 8.53 (t, J=5.2 Hz, 1H), 8.19 (t, J=5.6 Hz, 1H), 7.93 (d, J=8.6 Hz, 2H), 7.80-7.60 (m, 4H), 7.60-7.30 (m, 3H), 3.61 (d, J=5.6 Hz, 2H), 3.59-3.40 (m, 2H), 2.43 (t, J=7.4 Hz, 2H).

### Example 2(38)

N-hydroxy-5-[(4-phenylbenzoyl)aminomethyl]thiophene-2-carboxamide 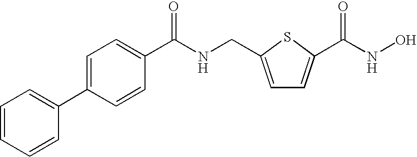
TLC: Rf 0.30 (chloroform:methanol=9:1);
NMR (DMSO-d₆): δ 11.20-11.00 (br, 1H), 9.23 (t, J=5.8 Hz, 1H), 9.18-8.98 (br, 1H), 7.97 (d, J=8.4 Hz, 2H), 7.78 (d, J=8.4 Hz, 2H), 7.73 (d, J=7.0 Hz, 2H), 7.56-7.32 (m, 4H), 7.01 (d, J=3.6 Hz, 1H), 4.63 (d, J=5.8 Hz, 2H).

### Example 2(39)

N-hydroxy-6-[(4-hydroxymethylbenzoyl)amino]hexanamide 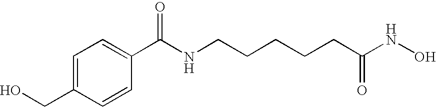
TLC: Rf 0.17 (ethyl acetate:methano=9:1);
NMR (DMSO-d₆): δ 10.32 (s, 1H), 8.64 (s, 1H), 8.37 (t, J=5.7 Hz, 1H), 7.78 (d, J=8.3 Hz, 2H), 7.36 (d, J=8.3 Hz, 2H), 5.27 (t, J=6.0 Hz, 1H), 4.52 (d, J=6.0 Hz, 2H), 3.21 (m, 2H), 1.93 (t, J=7.2 Hz, 2H), 1.55-1.45 (m, 4H), 1.30-1.22 (m, 2H).

### Example 2(40)

N-hydroxy-3-[(4-phenylbenzylcarbonyl)amino]propanamide 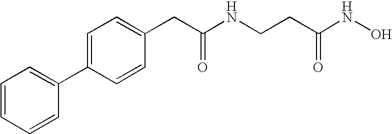
TLC: Rf 0.23 (chloroform:methanol:acetic acid=9:1:01);
NMR (DMSO-d₆): δ 10.38 (s, 1H), 8.20-8.00 (m,1H), 7.65-7.20 (m, 9H), 3.39 (s, 2H), 3.30-3.10 (m, 2H), 2.10 (t, J=7.0 Hz, 2H).

### Example 2(41)

N-hydroxy-6-[(4-phenylcyclohexylcarbonyl)amino]hexanamide 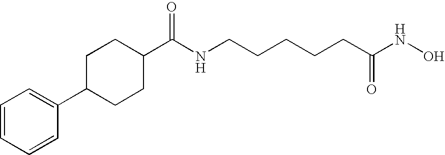
TLC: Rf 0.43 (chloroform:methanol:acetic acid=9:1:0.1);
NMR (DMSO-d₆): δ 10.50-10.05 (br, 1H), 8.90-8.40 (br, 1H), 7.80-7.50 (m, 1H), 7.38-7.00 (m, 5H), 3.10-2.85 (m, 2H), 2.60-1.00 (m, 18H, overlap with DMSO).

### Example 2(42)

N-hydroxy-6-{[4-(4-methoxyphenyl)benzoyl]amino}hexanamide 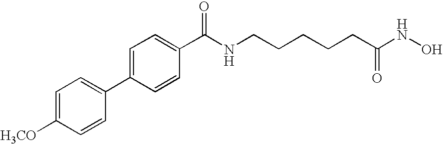
TLC: Rf 0.49 (chloroform:methanol:acetic acid=6:1:0.1);
NMR (DMSO-d₆): δ 10.28 (s, 1H), 8.80-8.45 (br, 1H), 8.42 (t, J=5.7 Hz, 1H), 7.86 (d, J=8.4 Hz, 2H), 7.67 (d, J=8.4 Hz, 2H), 7.64 (d, J=8.8 Hz, 2H), 7.00 (d, J=8.8 Hz, 2H), 3.78 (s, 3H), 3.30-3.10 (m, 2H), 1.91 (t, J=7.2 Hz, 2H), 1.60-1.38 (m, 4H), 1.38-1.10 (m, 2H).

### Example 2(43)

N-hydroxy-6-[(4-phenyl-3-cyclohexenylcarbonyl)amino]hexanamide 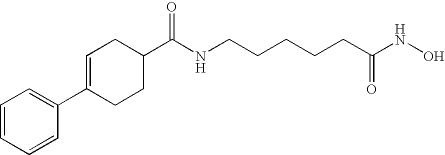
TLC: Rf 0.53 (chloroform:methanol:acetic acid=6:1:0.1);
NMR (DMSO-d₆): δ 10.32 (s, 1H), 7.77 (t, J=5.3 Hz, 1H), 7.45-7.10 (m, 5H), 6.18-6.05 (m, 1H), 3.15-2.85 (m, 2H), 2.50-2.05 (m, 5H), 2.00-1.78 (m, 3H), 1.78-1.00 (m, 7H).

### Example 2(44)

N-hydroxy-6-[(6-dimethylaminopyridine-3-yl)amino]hexanamide 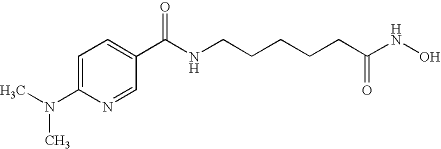
TLC: Rf 0.30 (chloroform:methanol:acetic acid=6:1:0.1);
NMR (DMSO-d₆): δ 10.30 (brs, 1H), 8.75-8.40 (m, 2H), 8.20-8.05 (m, 1H), 7.88 (dd, J=8.8, 2.2 Hz, 1H), 6.59 (d, J=8.8 Hz, 1H), 3.30-2.90 (m, 8H), 1.90 (t, J=7.0 Hz, 2H), 1.60-1.10 (m, 6H).

### Example 2(45)

N-hydroxy-6-{[4-(4-chlorophenyl)-3-hydroxymethylbenzoyl]amino}hexanamide 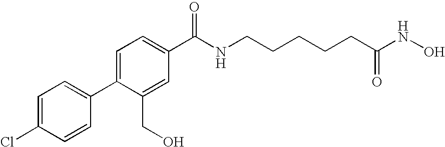
TLC: Rf 0.14 (chloroform:methanol:acetic acid=90:10:1);
NMR (DMSO-d₆): δ 10.31 (s, 1H), 8.64 (s, 1H), 8.49 (t, J=5.4 Hz, 1H), 8.05 (d, J=1.5 Hz, 1H), 7.77 (dd, J=7.8 Hz, 1.5 Hz, 1H), 7.50 (d, J=8.4 Hz, 2H), 7.42 (d, J=8.4 Hz, 2H), 7.29 (d, J 7.8 Hz, 1H), 5.26 (t, J=4.8 Hz, 1H), 4.39 (d, J=4.8 Hz, 2H), 3.30-3.23 (m, 2H), 1.93 (t, J=7.5 Hz, 2H), 1.58-1.43 (m, 4H), 1.32-1.22 (m, 2H).

### Example 2(46)

N-hydroxy-6-[(5-phenylpyrimidin-2-ylcarbonyl)amino]hexanamide 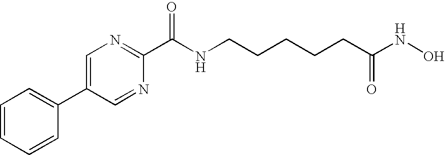
TLC: Rf 0.38 (chloroform:methanol:acetic acid=6:1:0.1);
NMR (DMSO-d₆+CD₃OD): δ 9.25 (s, 2H), 8.00-7.80 (m, 2H), 7.65-7.35 (m, 3H), 3.26 (t, J=7.0 Hz, 2H), 1.92 (t, J=7.4 Hz, 2H), 1.60-1.38 (m, 4H), 1.38-1.10 (m, 2H).

### Example 2(47)

N-hydroxy-6-[(4-cyclohexylphenyl)carbamoyl]hexanamide 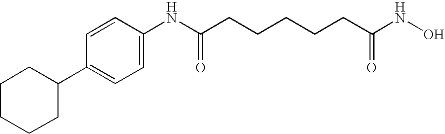
TLC: Rf 0.33 (chloroform:methanol:acetic acid=9:1:0.1);
NMR (DMSO-d₆): δ 10.32 (s, 1H), 9.74 (s, 1H), 8.64 (s, 1H), 7.46 (d, J=8.4 Hz, 2H), 7.10 (d, J=8.4 Hz, 2H), 2.60-2.30 (m, 1H, overlap with DMSO), 2.25 (t, J=7.3 Hz, 2H), 1.93 (t, J=6.9 Hz, 2H), 1.85-1.10 (m, 16H).

### Example 2(48)

N-hydroxy-6-[(3-phenylphenyl)carbamoyl]hexanamide 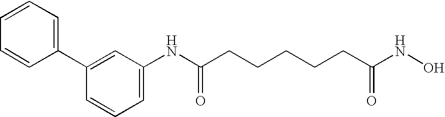
TLC: Rf 0.31 (chloroform:methanol:acetic acid=9:1:0.1);
NMR (DMSO-d₆): δ 10.33 (s, 1H), 9.94 (s, 1H), 8.65 (s, 1H), 7.92 (s, 1H), 7.63-7.22 (m, 8H), 2.31 (t, J=7.3 Hz, 2H), 1.95 (t, J=7.3 Hz, 2H), 1.70-1.40 (m, 4H), 1.40-1.20 (m, 2H).

### Example 2(49)

N-hydroxy-4-[(4-phenylbenzyl)carbamoyl]butanamide 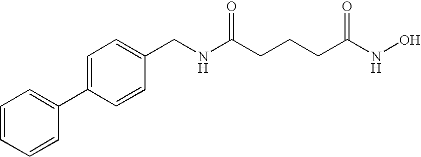
TLC: Rf 0.21 (chloroform:methanol:acetic acid=9:1:0.1);
NMR (DMSO-d₆): δ 10.35 (s, 1H), 8.62 (brs, 1H), 8.32 (t, J=5.8 Hz, 1H), 7.65-7.50 (m, 4H), 7.50-7.22 (m, 5H), 4.26 (d, J=5.8 Hz, 2H), 2.12 (t, J=7.2 Hz, 2H), 1.94 (t, J=7.2 Hz, 2H), 1.80-1.60 (m, 2H).

### Example 2(50)

N-hydroxy-6-[(2-hydroxy-5-phenylphenyl)carbamoyl]hexanamide 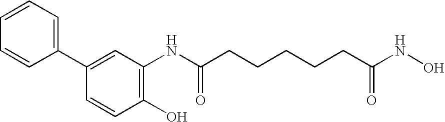
TLC: Rf 0.23 (chloroform:methanol:acetic acid=9:1:0.1);
NMR (DMSO-d₆): δ 10.30 (s, 1H), 9.93 (s, 1H), 9.26 (s, 1H), 8.63 (s, 1H), 8.03 (d, J=1.8 Hz, 1H), 7.60-7.15 (m, 6H), 6.89 (d, J=8.4 Hz, 1H), 2.37 (t, J=7.4 Hz, 2H), 1.91 (t, J=7.2 Hz, 2H), 1.70-1.38 (m, 4H), 1.38-1.10 (m, 2H).

### Example 2(51)

N-hydroxy-6-[4-(4-chlorophenyl)benzoyl]hexanamide 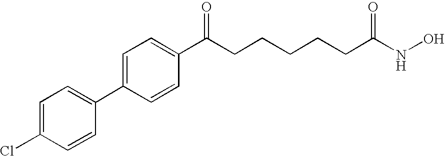
TLC: Rf 0.37 (ethyl acetate);
NMR (DMSO-d₆): δ 10.33 (s, 1H), 8.56 (s, 1H), 8.04 (d, J=8.5 Hz, 2H), 7.82 (d, J=8.5 Hz, 2H), 7.78 (d, J=8.5 Hz, 2H), 7.56 (d, j=8.5 Hz, 2H), 3.03 (t, J=7.2 Hz, 2H), 1.96 (t, J=7.2 Hz, 2H), 1.62 (quint, J=7.2 Hz, 2H), 1.54 (quint, J=7.2 Hz, 2H), 1.36-1.27 (m, 2H).

### Example 2(52)

N-hydroxy-4-[4-(4-chlorophenyl)benzoyl]butanamide 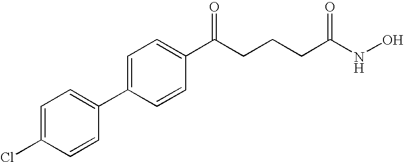
TLC: Rf 0.25 (ethyl acetate);
NMR (CDCl₃+DMSO-d₆): δ 10.36 (s, 1H), 8.58 (brs, 1H), 8.03 (d, J=8.4 Hz, 2H), 7.70-7.50 (m, 2H), 7.50-7.40 (m, 2H), 3.07 (t, J=7.0 Hz, 2H), 2.23 (t, J=6.8 Hz, 2H), 2.10 (m, 2H).

### Example 2(53)

N-hydroxy-7-[4-(4-chlorophenyl)benzoyl]heptanamide 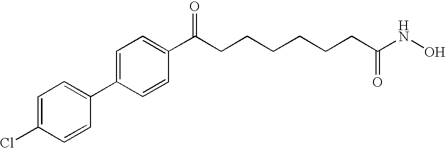
TLC: Rf 0.38 (ethyl acetate);
NMR (DMSO-d₆): δ 10.32 (s, 1H), 8.64 (s, 1H), 8.04 (d, J=8.5 Hz, 2H), 7.82 (d, J=8.5 Hz, 2H), 7.78 (d, J=8.5 Hz, 2H), 7.55 (d, J=8.5 Hz, 2H), 3.03 (t, J=7.2 Hz, 2H), 1.94 (t, J=7.2 Hz, 2H), 1.62 (quint, J=7.2 Hz, 2H), 1.49 (quint, J=7.2 Hz, 2H), 1.37-1.26 (m, 4H).

### Example 2(54)

N-hydroxy-5-(naphthalen-2-ylcarbonyl)pentanamide 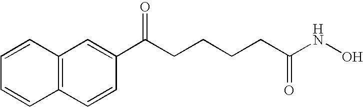
TLC: Rf 0.48 (ethyl acetate);
NMR (DMSO-d₆): δ 10.35 (s, 1H), 8.66 (s, 2H), 8.12 (d, J=7.2 Hz, 1H), 8.02-7.95 (m, 3H), 7.68-7.58 (m, 2H), 3.15 (t, J=6.6 Hz, 2H), 2.00 (t, J=6.6 Hz, 2H), 1.70-1.57 (m, 4H).

### Example 2(55)

(7E)-N-hydroxy-6-oxo-8-phenyloct-7-enamide 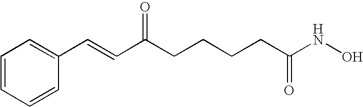
TLC: Rf 0.25 (ethyl acetate);
NMR (CDCl₃+DMSO-d₆): δ 10.38 (s, 1H), 8.67 (s, 1H), 7.65 (m, 2H), 7.58 (d, J=16.0 Hz, 1H), 7.42 (m, 3H), 6.82 (d, J=16.0 Hz, 1H), 2.66 (m, 2H), 2.02 (m, 2H), 1.60 (m, 4H).

### Example 2(56)

(7E, 9E)-N-hydroxy-6-oxo-10-phenyldec-7,9-dienamide 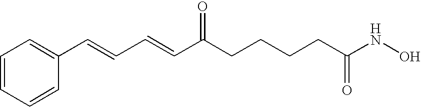
TLC: Rf 0.22 (ethyl acetate);
NMR (DMSO-d₆): δ 10.35 (s, 1H), 8.67 (s, 1H), 7.58 (m, 2H), 7.50-7.25 (m, 4H), 7.12 (m, 2H), 6.32 (d, J=15.4 Hz, 1H), 2.60 (m, 2H), 1.97 (m, 2H), 1.51 (m, 4H).

### Example 2(57)

N-hydroxy-5-(benzo[b]thiophen-2-ylcarbonyl)pentanamide 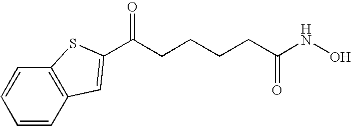
TLC: Rf 0.32 (ethyl acetate);
NMR (DMSO-d₆): δ 10.38 (s, 1H), 8.67 (s, 1H), 8.36 (s, 1H), 8.04-7.99 (m, 2H), 7.54-7.43 (m, 2H), 3.07 (t, J=7.2 Hz, 2H), 1.98 (t, J=7.2 Hz, 2H), 1.64-1.54 (m, 4H).

### Example 2(58)

N-hydroxy-5-benzo[b]thiophen-3-ylcarbonyl)pentanamide 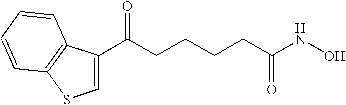
TLC: Rf 0.32 (ethyl acetate);
NMR (DMSO-d₆): δ 10.34 (s, 1H), 8.65 (s, 1H), 8.61-8.58 (m, 1H), 8.08-8.05 (m, 1H), 7.51-7.41 (m, 2H), 3.03 (t, J=7.2 Hz, 2H), 1.98 (t, J=7.2 Hz, 2H), 1.64-1.50 (m, 4H).

### Example 2(59)

N-hydroxy-5-(4-phenoxybenzoyl)pentanamide 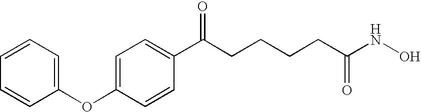
TLC: Rf 0.28 (ethyl acetate);
NMR (DMSO-d₆): δ 10.34 (s, 1H), 8.67 (s, 1H), 7.98 (d, J=8.7 Hz, 2H), 7.49-7.43 (m, 2H), 7.27-7.22 (m, 1H), 7.13-7.10 (m, 2H), 7.04 (d, J=8.7 Hz, 2H), 2.97 (t, J=6.9 Hz, 2H), 1.98 (t, J=6.9 Hz, 2H), 1.61-1.52 (m, 4H).

### Example 2(60)

N-hydroxy-4-[4-(4-chlorophenyl)benzoylmethylthio]butanamide 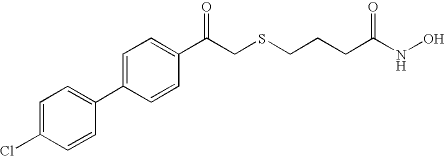
TLC: Rf 0.40 (chloroform:methanol=9:1);
NMR (DMSO-d₆): δ 10.37 (s, 1H), 8.68 (s, 1H), 8.06 (d, J=8.2 Hz, 2H), 7.82 (d, J=8.2 Hz, 2H), 7.78 (d, J=8.4 Hz, 2H), 7.55 (d, J=8.4 Hz, 2H), 4.01 (s, 2H), 2.56-2.42 (m, 2H), 2.03 (t, J=6.8 Hz, 2H), 1.82-1.64 (m, 2H).

### Example 2(61)

N-hydroxy-3-[4-(4-chlorophenyl)benzoylmethoxy]benzamide 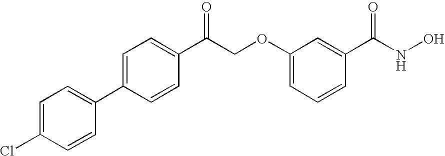
TLC: Rf 0.25 (chloroform:methanol=19:1);
NMR (DMSO-d₆): δ 11.30-11.00 (br, 1H), 9.16-8.90 (br, 1H), 8.01 (d, J=8.6 Hz, 2H), 7.88 (d, J=8.6 Hz, 2H), 7.81 (d, J=8.4 Hz, 2H), 7.57 (d, J=8.4 Hz, 2H), 7.40-7.28 (m, 3H), 7.20-7.07 (m, 1H), 5.66 (s, 2H).

### Example 2(62)

N-hydroxy-3-[4-(4-chlorophenyl)benzoylmethylthio]propanamide 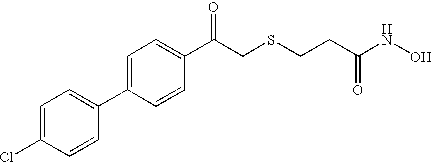
TLC: Rf 0.42 (chloroform:methanol=9:1);
NMR (DMSO-d₆): δ 10.42 (s, 1H), 8.79 (s, 1H), 8.06 (d, J=8.4 Hz, 2H), 7.83 (d, J=8.4 Hz, 2H), 7.79 (d, J=8.4 Hz, 2H), 7.55 (d, J=8.4 Hz, 2H), 4.05 (s, 2H), 2.69 (t, J=7.2 Hz, 2H), 2.26 (t, J=7.2 Hz, 2H).

### Example 2(63)

N-hydroxy-4-[4-(4-chlorophenyl)benzoylmethoxy]benzamide 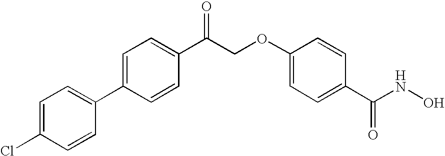
TLC: Rf 0.34 (chloroform:methanol=9:1);
NMR (DMSO-d₆): δ 11.07 (s, 1H), 9.50-8.40 (br, 1H), 8.10 (d, J=8.4 Hz, 2H), 7.88 (d, J=8.4 Hz, 2H), 7.80 (d, J=8.8 Hz, 2H), 7.71 (d, J=8.8 Hz, 2H), 7.57 (d, J=8.8 Hz, 2H), 7.03 (d, J=8.8 Hz, 2H), 5.69 (s, 2H).

### Example 2(64)

N-hydroxy-4-[4-(4-chlorophenyl)benzyloxy]butanamide 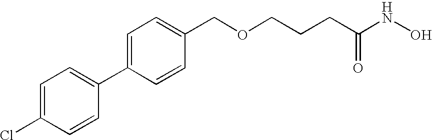
TLC: Rf 0.24 (ethyl acetate);
NMR (DMSO-d₆): δ 10.36 (s, 1H), 8.66 (s, 1H), 7.70-7.61 (m, 4H), 7.51-7.34 (m, 4H), 4.47 (s, 2H), 3.42 (t, J=6.3 Hz, 2H), 2.03 (t, J=6.3 Hz, 2H), 1.76 (m, 2H).

### Example 2(65)

N-hydroxy-5-[4-(4-chlorophenyl)-2-hydroxymethylphenoxy]pentanamide 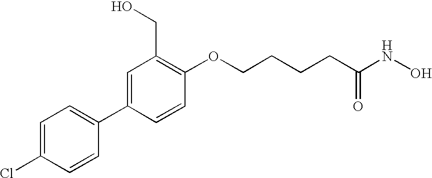
TLC: Rf 0.30 (chloroform:methanol=9:1);
NMR (DMSO-d₆): δ 10.37 (s, 1H), 8.69 (s, 1H), 7.78-7.57 (m, 3H), 7.56-7.41 (m, 3H), 7.01 (d, J=8.6 Hz, 1H), 5.20-5.00 (m, 1H), 4.54 (s, 2H), 4.08-3.92 (m, 2H), 2.06-1.94 (m, 2H), 1.82-1.56 (m, 4H).

### Example 2(66)

N-hydroxy-5-[4-(4-chlorophenyl)-2-hydroxyphenoxy]pentanamide 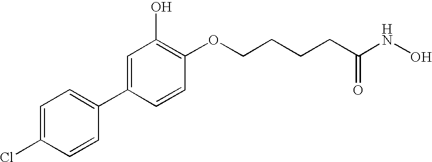
TLC: Rf 0.34 (chloroform:methanol=9:1);
NMR (DMSO-d₆): δ 10.20-8.50 (br, 3H), 7.56 (d, J=8.8 Hz, 2H), 7.44 (d, J=8.8 Hz, 2H), 7:08-6.92 (m, 3H), 4.02-3.90 (m, 2H), 2.07-1.95 (m, 2H), 1.80-1.56 (m, 4H).

### Example 2(67)

N-hydroxy-5-[4-(4-cyanophenyl)phenoxy]pentanamide 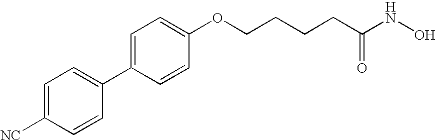
TLC: Rf 0.35 (ethyl acetate);
NMR (CDCl₃+DMSO-d₆): δ 10.20 (s, 1H), 8.40 (brs, 1H), 7.75-7.60 (m, 4H), 7.53 (dd, J=6.6 Hz and 2.0 Hz, 2H), 6.98 (dd, J=6.6 Hz and 2.0 Hz, 2H), 4.02 (t, J=5.4 Hz, 2H), 2.21 (t, J=6.4 Hz, 2H), 1.85 (m, 4H).

### Example 2(68)

N-hydroxy-6-[4-(4-cyanophenyl)phenoxy]hexanamide 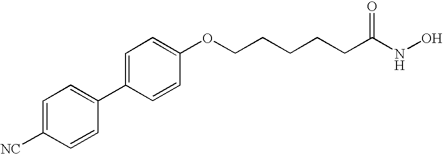
TLC: Rf 0.30 (ethyl acetate);
NMR (DMSO-d₆): δ 10.35 (s, 1H), 8.60 (brs, 1H), 7.88 (d, J=8.8 Hz, 2H), 7.82 (d, J=8.8 Hz, 2H), 7.70 (d, J=8.8 Hz, 2H), 7.05 (d, J=8.8 Hz, 2H), 4.02 (t, J=6.5 Hz, 2H), 1.99 (t, J=7.0 Hz, 2H), 1.74 (m, 2H), 1.57 (m, 2H), 1.43 (m, 2H).

### Example 2(69)

N-hydroxy-5-[4-(4-chlorophenyl)phenoxy]pentanamide 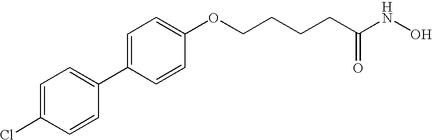
TLC: Rf 0.47 (chloroform:methanol:acetic acid=10:1:1);
NMR (DMSO-d₆): δ 10.38 (s, 1H), 8.69 (s, 1H), 7.65-7.55 (m, 4H), 7.45 (dd, J=6.6 Hz and 2.1 Hz, 2H), 7.01 (dd, J=6.9 Hz and 1.8 Hz, 2H), 4.01 (t, J=6.0 Hz, 2H), 2.03 (t, J=6.8 Hz, 2H), 1.80-1.60 (m, 4H).

### Example 2(70)

N-hydroxy-7-[4-(4-cyanophenyl)phenoxy]heptanamide 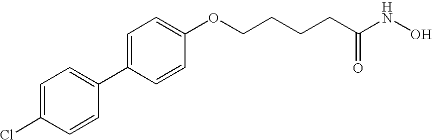
TLC: Rf 0.32 (ethyl acetate);
NMR (CDCl₃+DMSO-d₆): δ 9.90 (brs, 1H), 8.20 (brs, 1H), 7.75-7.60 (m, 4H), 7.53 (dd, J=6.6 Hz and 2.1 Hz, 2H), 6.98 (dd, J=6.6 Hz and 2.1 Hz, 2H), 4.00 (t, J=6.4 Hz, 2H), 2.14 (t, J=7.5 Hz, 2H), 1.80 (m, 2H), 1.70 (m, 2H), 1.45 (m, 4H).

### Example 2(71)

N-hydroxy-6-[4-(4-chlorophenyl)phenoxy]hexanamide 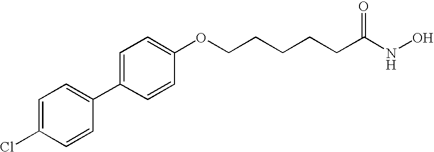
TLC: Rf 0.48 (chloroform:methanol:acetic acid=10:1:1);
NMR (CDCl₃+DMSO-d₆): δ 10.24 (brs, 1H), 8.50 (brs, 1H), 7.55-7.42 (m, 4H), 7.37 (dd, J=6.6 Hz and 2.1 Hz, 2H), 6.94 (dd, J=6.9 Hz and 2.1 Hz, 2H), 3.99 (t, J=6.4 Hz, 2H), 2.14 (t, J=7.5 Hz, 2H), 1.80 (m, 2H), 1.75 (m, 2H), 1.50 (m, 2H).

### Example 2(72)

N-hydroxy-7-[4-(4-chlorophenyl)phenoxy]heptanamide 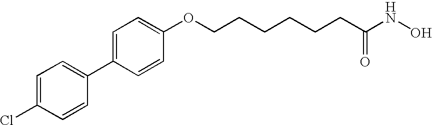
TLC: Rf 0.50 (chloroform:methanol:acetic acid=10:1:1);
NMR (CDCl₂+DMSO-d₆): δ 10.26 (s, 1H), 8.50 (brs, 1H), 7.60-7.42 (m, 4H), 7.37 (d, J=8.4 Hz, 2H), 6.95 (d, J=8.7 Hz, 2H), 3.98 (t, J=6.4 Hz, 2H), 2.10 (t, J=7.5 Hz, 2H), 1.80 (m, 2H), 1.65 (m, 2H), 1.40 (m, 4H).

### Example 2(73)

N-hydroxy-5-[4-(4-chlorophenyl)phenylthio]pentanamide 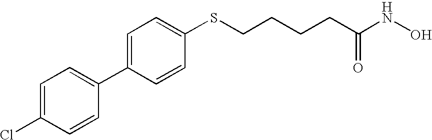
TLC: Rf 0.28 (ethyl acetate);
NMR (DMSO-d₆): δ 10.34 (s, 1H), 8.68 (s, 1H), 7.68 (d, J=8.5 Hz, 2H), 7.61 (d, J=8.5 Hz, 2H), 7.50 (d, J=8.5 Hz, 2H), 7.39 (d, J=8.5 Hz, 2H), 3.00 (t, J=6.9 Hz, 2H), 1.97 (t, J=6.9 Hz, 2H), 1.69-1.53 (m, 4H).

### Example 2(74)

N-hydroxy-7-[4-(4-chlorophenyl)phenylthio]heptanamide 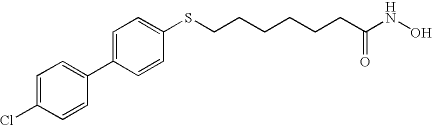
TLC: Rf 0.32 (ethyl acetate);
NMR (DMSO-d₆): δ 10.32 (s, 1H), 8.64 (s, 1H), 7.68 (d, J=8.5 Hz, 2H), 7.61 (d, J=8.5 Hz, 2H), 7.49 (d, J=8.5 Hz, 2H), 7.38 (d, J=8.5 Hz, 2H), 2.99 (t, J=7.2 Hz, 2H), 1.93 (t, J=7.2 Hz, 2H), 1.64-1.35 (m, 6H), 1.30-1.20 (m, 2H).

### Example 2(75)

N-hydroxy-4-[4-(4-chlorophenyl)benzylthio]butanamide 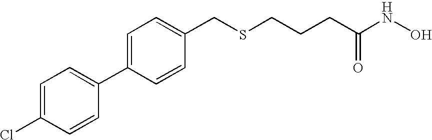
TLC: Rf 0.56 (chloroform:methanol=9:1);
NMR (DMSO-d₆): δ 10.36 (s, 1H), 8.67 (s, 1H), 7.70-7.55 (m, 4H), 7.52-7.29 (m, 5H), 3.75 (s, 2H), 2.40 (t, J=7.0 Hz, 2H), 2.03 (t, J=7.0 Hz, 2H), 1.82-1.64 (m, 2H).

### Example 2(76)

N-hydroxy-4-{[4-(4-chlorophenyl)phenylsulfonyl]amino}butanamide 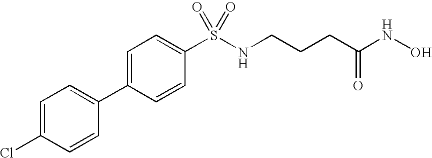
TLC: Rf 0.12 (ethyl acetate);
NMR (DMSO-d₆): δ 10.32 (s, 1H), 8.67 (s, 1H), 7.90 (d, J=8.4 Hz, 2H), 7.85 (d, J=8.4 Hz, 2H), 7.78 (d, J=8.4 Hz, 2H), 7.69 (t, J=5.7 Hz, 1H), 7.57 (d, J=8.4 Hz, 2H), 2.74 (m, 2H), 1.95 (t, J=7.2 Hz, 2H), 1.61 (quint, J=7.2 Hz, 2H).

### Example 2(77)

N-hydroxy-6-{[4-(4-chlorophenyl)phenylsulfonyl]amino}hexanamide 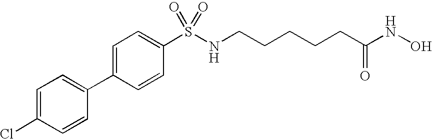
TLC: Rf 0.24 (ethyl acetate);
NMR (DMSO-d₆): δ 10.30 (s, 1H), 8.64 (s, 1H), 7.89 (d, J=9.0 Hz, 2H), 7.85 (d, J=9.0 Hz, 2H), 7.77 (d, J=8.5 Hz, 2H), 7.62 (t, J=5.7 Hz, 1H), 7.56 (d, J=8.5 Hz, 2H), 2.73 (m, 2H), 1.89 (t, J=7.5 Hz, 2H), 1.46-1.33 (m, 4H), 1.25-1.16 (m, 2H).

### Example 2(78)

N-hydroxy-5-[4-(4-chlorophenyl)phenylsulfinyl]pentanamide 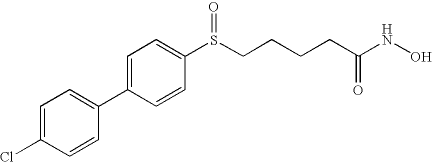
TLC: Rf 0.31 (ethyl acetate:methanol=9:1);
NMR (DMSO-d₆): δ 10.34 (s, 1H), 8.62 (br, 1H), 7.93 (d, J=8.7 Hz, 2H), 7.77 (d, J=8.7 Hz, 2H), 7.74 (d, J=8.7 Hz, 2H), 7.55 (d, J=8.7 Hz, 2H), 3.05-2.95 (m, 1H), 2.85-2.77 (m, 1H), 1.96 (t, J=6.7 Hz, 2H), 1.70-1.42 (m, 4H).

### Example 2(79)

N-hydroxy-7-[4-(4-chlorophenyl)phenylsulfinyl]heptanamide 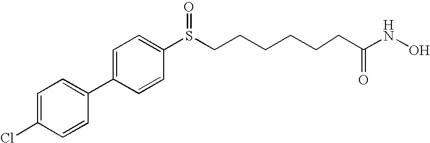
TLC: Rf 0.34 (ethyl acetate:methanol=9:1);
NMR (DMSO-d₆): δ 10:31 (s, 1H), 7.87 (d, J=8.4 Hz, 2H), 7.76 (d, J=8.4 Hz, 2H), 7.73 (d, J=8.4 Hz, 2H), 7.54 (d, J=8.4 Hz, 2H), 3.02-2.93 (m, 1H), 2.83-2.74 (m, 1H), 1.92 (t, J=7.3 Hz, 2H), 1.72-1.60 (m, 1H), 1.50-1.20 (m, 7H).

### Example 2(80)

N-hydroxy-5-[4-(4-chlorophenyl)phenylsulfonyl]pentanamide 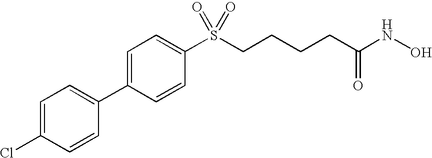
TLC: Rf 0.18 (ethyl acetate);
NMR (DMSO-d₆): δ 10.31 (s, 1H), 8.68 (s, 1H), 7.96 (s, 4H), 7.81 (d, J=8.8 Hz, 2H), 7.58 (d, J=8.8 Hz, 2H), 3.42-3.30 (m, 2H), 1.94 (m, 2H), 1.60-1.52 (m, 4H).

### Example 2(81)

N-hydroxy-7-[4-(4-chlorophenyl)phenylsulfonyl]heptanamide 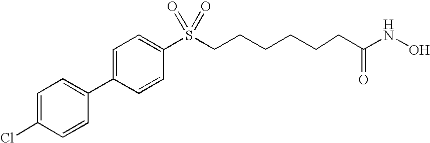
TLC: Rf 0.21 (ethyl acetate);
NMR (DMSO-d₆): δ 10.30 (br, 1H), 7.96 (s, 4H), 7.80 (d, J=8.8 Hz, 2H), 7.58 (d, J=8.8 Hz, 2H), 3.32 (t, J=7.7 Hz, 2H), 1.90 (t, J=7.0 Hz, 2H), 1.63-1.20 (m, 8H).

### Example 2(82)

N-hydroxy-6-methoxy-6-(4-phenoxyphenyl)hexanamide 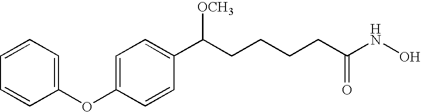
TLC: Rf 0.37 (ethyl acetate);
NMR (CDCl₃): δ 7.36-7.30 (m, 2H), 7.20 (d, J=8.7 Hz, 2H), 7.12-7.07 (m, 1H), 7.03-6.99 (m, 2H), 6.96 (d, J=8.7 Hz, 2H), 4.06 (dd, J=7.2, 5.7 Hz, 1H), 3.18 (s, 3H), 2.12 (t, J=7.0 Hz, 2H), 1.87-1.72 (m, 1H), 1.68-1.56 (m, 3H), 1.50-1.26 (m, 2H).

### Example 2(83)

N-hydroxy-6-methoxy-6-[4-(morpholin-4-yl)phenyl]hexanamide 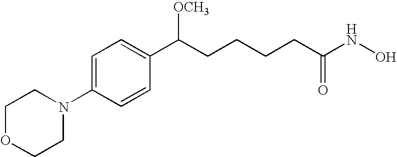
TLC: Rf 0.40 (chloroform:methanol=9:1);
NMR (DMSO-d₆): δ 10.27 (s, 1H), 8.61 (s, 1H), 7.10 (d, J=8.7 Hz, 2H), 6.89 (d, J=8.7 Hz, 2H), 3.96 (t, J=6.6 Hz, 1H), 3.73-3.69 (m, 4H), 3.09-3.05 (m, 4H), 3.02 (s, 3H), 1.87 (t, J=7.2 Hz, 2H), 1.73-1.60 (m, 1H), 1.54-1.38 (m, 3H), 1.30-1.04 (m, 2H).

### Example 2(84)

N-hydroxy-6-methoxymethoxy-6-[4-(4-chlorophenyl)phenyl]-2-methylhexanamide 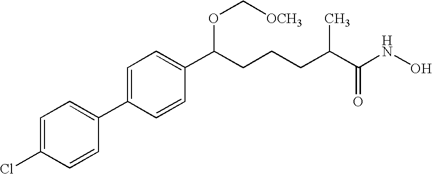
TLC: Rf 0.40 (ethyl acetate)
NMR (DMSO-d₆): δ 10.34 (s, 1H), 8.65 (s, 1H), 7.70-7.61 (m, 4H), 7.51-7.32 (m, 4H), 4.55-4.49 (m, 2H), 4.42-4.40 (m, 1H), 3.25 (s, 3H), 2.04-1.99 (m, 1H), 1.78-1.43 (m, 3H), 1.35-1.17 (m, 3H), 0.95 and 0.94 (each d, J=6.6 Hz, 3H).

### Example 2(85)

(3E)-N-hydroxy-6-methoxymethoxy-6-[4-(4-chlorophenyl)phenyl]hex-3-enamide 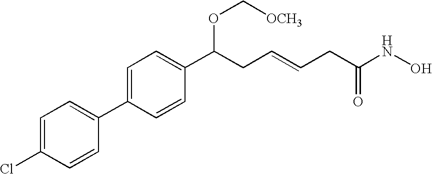
TLC: Rf 0.32 (chloroform:methanol=9:1);
NMR (DMSO-d₆): δ 10.36 (s, 1H), 8.71 (brs, 1H), 7.68 (d, J=8.4 Hz, 2H), 7.64 (d, J=8.4 Hz, 2H), 7.49 (d, J=8.4 Hz, 2H), 7.41 (d, J=8.4 Hz, 2H), 5.57-5.44 (m, 2H), 4.61 (dd, J=7.5 Hz, 5.7 Hz, 1H), 4.53 (d, J=6.6 Hz, 1H), 4.43 (d, J=6.6 Hz, 1H), 3.24 (s, 3H), 2.71 (d, J=5.7 Hz, 2H), 2.49-2.39 (m, 2H).

### Example 2(86)

N-hydroxy-4-{1-[4-(4-chlorophenyl)phenyl]-2-(methoxymethoxy)ethoxy}butanamide 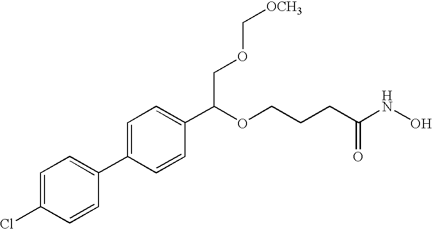
TLC: Rf 0.20 (ethyl acetate);
NMR (DMSO-d₆): δ 10:32 (br, 1H), 8.65 ), (br, 1H), 7.69 (d, J=8.3 Hz, 2H), 7.64 (d, J=8.3 Hz, 2H), 7.50 (d, J=8.3 Hz, 2H), 7.41 (d, J=8.3 Hz, 2H), 4.57 (d, J=6.6 Hz, 1H), 4.55 (d, J=6.6 Hz, 1H), 4.50 (dd, J=7.0, 4.5 Hz, 1H), 3.63 (dd, J=10.5, 7.0 Hz, 1H), 3.52 (dd, J=10.5, 4.5 Hz, 1H), 3.29-3.24 (m, 2H), 3.18 (s, 3H), 2.03-1.97 (m, 2H), 1.76-1.67 (m, 2H).

### Example 2(87)

N-methoxy-6-benzyloxymethoxy-6-[4-(4-chlorophenyl)phenyl]hexanamide 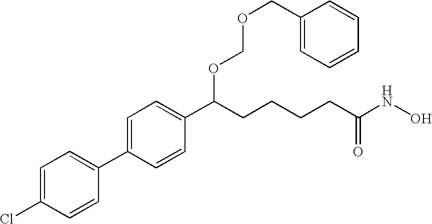
TLC: Rf 0.48 (ethyl acetate);
NMR (DMSO-d₆): δ 10.29 (br, 1H), 8.65 (br, 1H), 7.68 (d, J=8.4 Hz, 2H), 7.63 (d, J=8.4 Hz, 2H, 7.49 (d, J=8.4 Hz, 2H), 7.40 (d, J=8.4 Hz, 2H), 7.33-7.22 (m, 5H), 4.70 (d, J=6.9 Hz, 1H), 4.64-4.59 (m, 1H), 4.58 (d, J=11.7 Hz, 1H), 4.54 (d, J=6.9 Hz, 1H), 4.44 (d, J=11.7 Hz, 1H), 1.91 (t, J=7.2 Hz, 2H), 1.83-1.71 (m, 1H), 1.68-1.56 (m, 1H), 1.50 (m, 2H), 1.42-1.30 (m, 1H), 1.29-1.16 (m, 1H).

### Example 2(88)

N-hydroxy-6-(4-methoxyphenyl)-6-(4-phenylphenyl)hexanamide 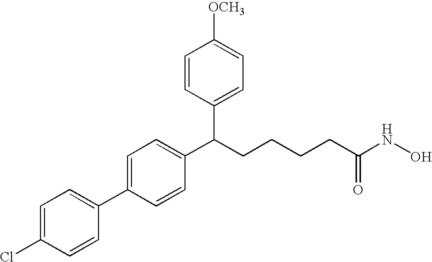
TLC: Rf 0.31 (ethyl acetate);
NMR (DMSO-d₆): δ 10.28 (br, 1H), 8.64 (br, 1H), 7.60 (d, J=8.4 Hz, 2H), 7.53 (d, J=8.4 Hz, 2H), 7.44-7.39 (m, 2H), 7.35-7.29 (m, 3H), 7.22 (d, J=8.4 Hz, 2H), 6.83 (d, J=8.4 Hz, 2H), 3.87 (t, J=7.5 Hz, 1H), 3.68 (s, 3H), 2.30-1.96 (m, 2H), 1.89 (t, J=7.5 Hz, 2H), 1.52 (m, 2H), 1.23-1.15 (m, 2H).

### EXAMPLE 2(89)

(6R)-N-hydroxy-6-[4-(4-ethylphenyl)phenyl]-6-methoxyhexanamide 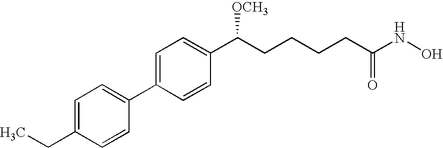
TLC: Rf 0.44 (chloroform:methanol=9:1);
NMR (DMSO-d₆): δ 10:30 (s, 1H), 7.61 (d, J=8.4 Hz, 2H), 7.57 (d, J=8.4 Hz, 2H), 7.33 (d, J=8.8 Hz, 2H), 7.28 (d, J=8.8 Hz, 2H), 4.13 (t, J=6.4 Hz, 1H), 3.11 (s, 3H), 2.63 (q, J=7.4 Hz, 2H), 1.91 (t, J=7.0 Hz, 2H), 1.81-1.21 (m, 6H), 1.20 (t, J=7.4 Hz, 2H).

### Example 2(90)

N-hydroxy-6-[4-(4-chlorophenyl)phenyl]-6-methoxymethoxyhexanamide 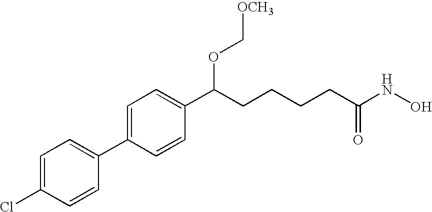
TLC: Rf 0.35 (ethyl acetate)
NMR (DMSO-d₆): δ 10.30 (s, 1H), 7.69 (d, J=8.5 Hz, 2H), 7.64 (d, J=8.5 Hz, 2H), 7.50 (d, J=8.5 Hz, 2H), 7.39 (d, J=8.5 Hz, 2H), 4.58-4.56 (m, 1H), 4.52 (d, J=6.6 Hz, 1H), 4.43 (d, J=6.6 Hz, 1H), 3.26 (s, 3H), 1.92 (t, J=7.3 Hz, 2H), 1.83-1.44 (m, 4H), 1.42-1.18 (m, 2H).

### Example 2(91)

N-hydroxy-6-[4-(4-chlorophenyl)phenyl]-6-methoxyhexanamide 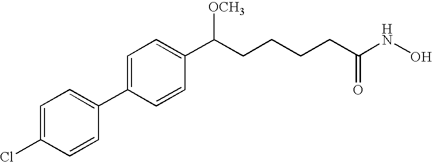
TLC: Rf 0.36 (ethyl acetate);
NMR (DMSO-d₆): δ 10.30 (s, 1H), 8.64 (s, 1H), 7.70 (d, J=8.5 Hz, 2H), 7.65 (d, J=8.5 Hz, 2H), 7.50 (d, J=8.5 Hz, 2H), 7.36 (d, J=8.5 Hz, 2H), 4.16 (t, J=6.5 Hz, 1H), 3.12 (s, 3H), 1.91 (t, J=7.2 Hz, 2H), 1.78-1.66 (m, 1H), 1.62-1.44 (m, 3H), 1.38-1.14 (m, 2H).

### Example 2(92)

N-hydroxy-4-[4-(4-chlorophenyl)phenyl]-4-methoxymethoxybutanamide 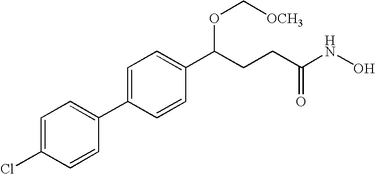
TLC: Rf 0.24 (ethyl acetate);
NMR (CDCl₃): δ 8.80 (brs, 1H), 7.60-7.45 (m, 4H), 7.45-7.30 (m, 4H), 4.63 (dd, J=8.1 Hz and 5.4 Hz, 1H), 4.59 (d, J=6.6 Hz, 1H), 4.53 (d, J=6.6 Hz, 1H), 3.38 (s, 3H), 2.31 (m, 2H), 2.07 (m, 2H).

### Example 2(93)

N-hydroxy-5-[4-(4-chlorophenyl)phenyl]-5-methoxymethoxypentanamide 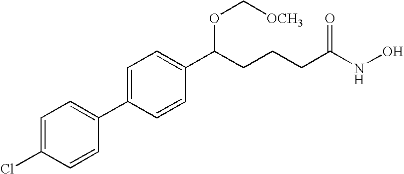
TLC: Rf 0.30 (ethyl acetate);
NMR (CDCl₃+DMSO-d₆): δ 10.28 (s, 1H), 8.51 (brs, 1H), 7.60-7.50 (m, 4H), 7.45-7.30 (m, 4H), 4.61 (m, 1H), 4.53 (s, 2H), 3.35 (s, 3H), 2.99 (s, 3H), 2.11 (t, J=6.6 Hz, 2H), 2.00-1.55 (m, 4H).

### Example 2(94)

N-hydroxy-5-{2-[4-(4-chlorophenyl)phenyl]-1,3-dioxolan-2-yl}pentanamide 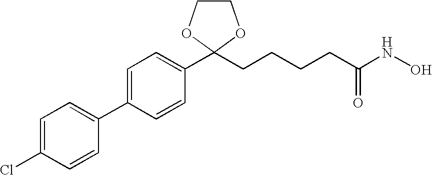
TLC: Rf 0.53 (ethyl acetate)
NMR (DMSO-d₆): δ 10.26 (s, 1H), 8.63 (s, 1H), 7.68 (d, J=8.7 Hz, 2H), 7.63 (d, J=8.7 Hz, 2H), 7.50 (d, J=8.7 Hz, 2H), 7.44 (d, J=8.7 Hz, 2H), 3.98-3.93 (m, 2H), 3.70-3.65 (m, 2H), 1.89-1.79 (m, 4H), 1.49-1.38 (m, 2H), 1.26-1.16 (m, 2H).

### Example 2(95)

N-hydroxy-4-{2-[4-(4-chlorophenyl)phenyl]-1,3-dioxolan-2-ylmethoxy}benzamide 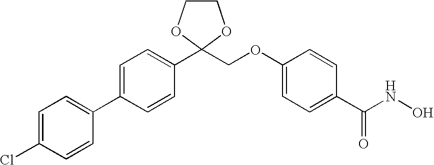
TLC: Rf 0.40 (chloroform:methanol=9:1);
NMR (DMSO-d₆): δ 11.04 (s, 1H), 8.88 (s, 1H), 7.78-7.64 (m, 6H), 7.61 (d, J=8.7 Hz, 2H), 7.51 (d, J=8.4 Hz, 2H), 6.98 (d, J=9.0 Hz, 2H), 4.26 (s, 2H), 4.18-4.03 (m, 2H), 3.95-3.81 (m, 2H).

### Example 2(96)

N-hydroxy-5-{2-[4-(4-chlorophenyl)phenyl]-4-methoxymethyl-1,3-dioxolan-2-yl}pentanamide 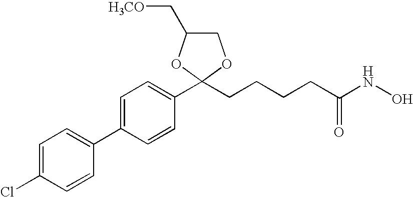
TLC: Rf 0.26 (ethyl acetate);
NMR (DMSO-d₆): δ 10.26 (brs, 1H), 8.63 (brs, 1H), 7.71-7.63 (m, 4H), 7.51-7.42 (m, 4H), 4.34-3.99 (m, 1H), 3.75-3.65 (m, 15H), 3.46-3.16 (m, 2.5H), 3.29 and 3.20 (each-s, 3H), 1.86 (m, 2H), 1.80 (m, 2H), 1.44 (m, 2H), 1.23 (m, 2H).

### Example 2(97)

N-hydroxy-5-{2-[4-(4-chlorophenyl)phenyl]-4-(4-hydroxybutyl)-1,3-dioxolan-2-yl}pentanamide 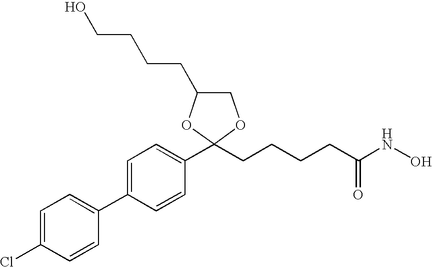
TLC: Rf 0.34 (ethyl acetate:methanol=9:1);
NMR (DMSO-d₆): δ 10.26 (brs, 1H), 8.63 (brs, 1H), 7.71-7.62 (m, 4H), 7.51-7.42 (m, 4H), 4.35 (br, 1H), 4.18-4.08 (m, 0.5H), 3.87-3.78 (m, 1.5H), 3.55-3.33 (m, 3H), 1.88 (m, 2H), 1.80 (m, 2H), 1.64-1.23 (m, 10H).

### Example 2(98)

N-hydroxy-6-hydroxy-6-(4-methylphenyl)hexanamide 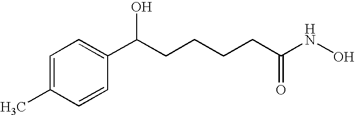
TLC: Rf 0.47 (ethyl acetate:methanol=39:1);
NMR (DMSO-d₆): δ 10.27 (brs, 1H), 8.62 (brs, 1H), 7.16 (d, J=8.1 Hz, 2H), 7.08 (d, J=8.1 Hz, 2H), 4.99 (d, J=4.5 Hz, 1H), 4.40-4.39 (m, 1H), 2.25 (s, 3H), 1.88 (t, J=7.2 Hz, 2H), 1.61-1.39 (m, 4H), 1.37-1.05 (m, 2H).

### Example 2(99)

N-hydroxy-6-hydroxy-6-phenylhexanamide 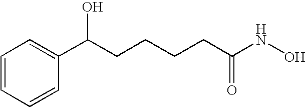
TLC: Rf 0.25 (ethyl acetate);
NMR (DMSO-d₆): δ 10.28 (s, 1H), 8.62 (s, 1H), 7.29-7.28 (m, 4H), 7.25-7.16 (m, 1H), 5.08 (d, J=1.8 Hz, 1H), 4.49-4.44 (m, 1H), 1.89 (t, J=7.5 Hz, 2H), 1.59-1.34 (m, 4H), 1.33-1.14 (m, 2H).

### Example 2(100)

N-hydroxy-6-[4-(4-chlorophenyl)phenyl]-2,2-dimethyl-6-hydroxyhexanamide 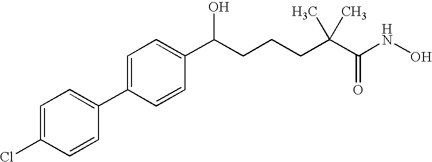
TLC: Rf 0.30 (ethyl acetate);
NMR (CDCl₃): δ 7.60-7.45 (m, 4H), 7.45-7.30 (m, 4H), 4.85-4.70 (m, 1H), 1.90-1.20 (m, 6H), 1.20 (s, 6H).

### Example 2(101)

(2E)-N-hydroxy-5-{3-[(phenylsulfonyl)amino]phenyl}pent-2-en-4-ynamide 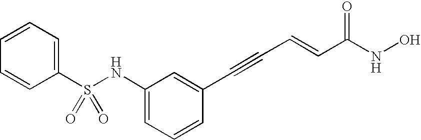
TLC: Rf 0.19 (chloroform:methanol=9:1);
NMR (DMSO-d₆): δ 10.82 (brs, 1H), 10.49 (brs, 1H), 9.19 (brs, 1H), 7.75-7.73 (m, 2H), 7.63-7.51 (m, 3H), 7.29-7.23 (m, 1H, 7.14-7.13 (m, 3H), 6.70 (d, J=15.9 Hz, 1H), 6.31 (d, J=15.9 Hz, 1H).

### Example 2(102)

N-hydroxy-4-[4-(4-chlorophenyl)phenyl]-4-hydroxybutanamide 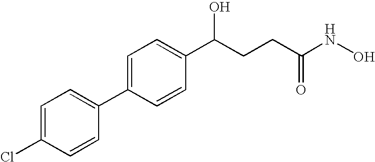
TLC: Rf 0.15 (ethyl acetate);
NMR (CDCl₃+DMSO-d₆); δ 10.30 (s, 1H), 8.50 (brs, 1H), 7.60-7.45 (m, 4H), 7.45-7.35 (m, 4H), 4.74 (dd, J=8.1 Hz and 4.2 Hz, 1H), 2.30 (m, 2H), 2.00 (m, 2H).

### Example 2(103)

N-hydroxy-2-{3-[4-(4-chlorophenyl)phenyl]propylthio}acetamide 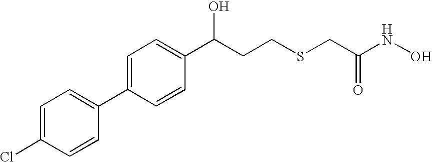
TLC: Rf 0.22 (ethyl acetate);
NMR (DMSO-d₆): δ 10.54 (s, 1H), 8.90 (brs, 1H), 7.68 (d, J=8.0 Hz, 2H), 7.62 (d, J=8.0 Hz, 2H), 7.49 (d, J=8.0 Hz, 2H), 7.42 (d, J=8.0 Hz, 2H), 5.50-5.20 (m, 1H), 4.67 (t, J=6.3 Hz, 1H), 3.00 (s, 2H), 2.65 (t, J=7.5 Hz, 2H), 1.90-1.80 (m, 2H).

### Example 2(104)

N-hydroxy-2-{3-hydroxy-3-[4-(4-chlorophenyl)phenyl]propylthio}propanamide 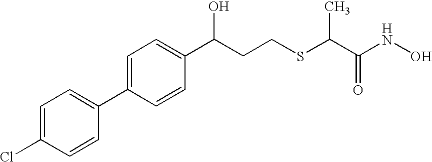
TLC: Rf 0.30 (ethyl acetate);
NMR (DMSO-d₆): δ 10.58 (brs, 1H), 8.91 (brs, 1H), 7.68 (d, J=8.5 Hz, 2H), 7.62 (d, J=8.5 Hz, 2H), 7.50 (d, J=8.5 Hz, 2H), 7.40 (d, J=8.5 Hz, 2H), 5.33 (d, J=2.8 Hz, 1H), 4.70-4.60 (m, 1H), 3.23 (q, J=7.2 Hz, 1H), 2.70-2.50 (m, 2H), 1.90-1.75 (m, 2H), 1.28 (d, J=7.2 Hz, 3H).

### Example 2(105)

N-hydroxy-7-hydroxy-7-[4-(4-chlorophenyl)phenyl]heptanamide 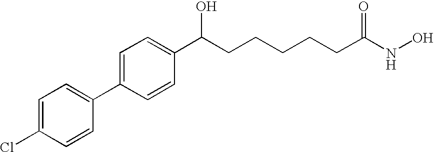
TLC: Rf 0.30 (ethyl acetate);
NMR (DMSO-d₆): δ 10.30 (s, 1H), 8.63 (s, 1H), 7.68 (d, J=8.5 Hz, 2H), 7.60 (d, J=8.5 Hz, 2H), 7.49 (d, J=8.5 Hz, 2H), 7.39 (d, J=8.5 Hz, 2H), 5.14 (d, J=4.5 Hz, 1H), 4.53 (m, 1H), 1.91 (t, J=7.3 Hz, 2H), 1.65-1.55 (m, 2H), 1.51-1.42 (m, 2H), 1.38-1.20 (m, 4H).

### Example 2(106)

N-hydroxy-8-[4-(4-chlorophenyl)phenyl]-8-hydroxy]octanamide 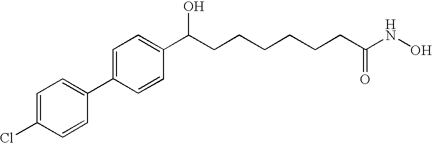
TLC: Rf 0.37 (ethyl acetate);
NMR: (DMSO-d₆): δ 10.31 (s, 1H), 8.64 (s, 1H), 7.68 (d, J=8.4 Hz, 2H), 7.60 (d, J=8.4 Hz, 2H), 7.49 (d, J=8.4 Hz, 2H), 7.39 (d, J=8.4 Hz, 2H), 5.14 (d, J=4.2 Hz, 1H), 4.54 (m, 1H), 1.92 (t, J=7.2 Hz, 2H), 1.66-1.54 (m, 2H), 1.51-1.41 (m, 2H), 1.35-1.19 (m, 6H).

### Example 2(107)

N-hydroxy-2-{3-[4-(4-chlorophenyl)phenyl]-3-hydroxypropylthio}-2-methylpropanamide 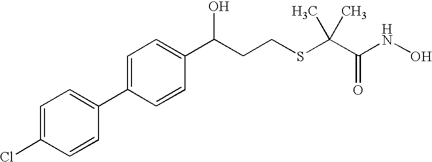
TLC: Rf 0.40 (ethyl acetate);
NMR (DMSO-d₆): δ 10.54 (brs, 1H), 8.70 (br.s., 1H), 7.68 (d, J=8 Hz, 2H), 7.62 (d, J=8 Hz, 2H), 7.49 (d, J=8 Hz, 2H), 7.41 (d, J=8 Hz, 2H), 5.30 (brs, 1H), 4.64 (t, J=6.2 Hz, 2H), 2.62 (t, J=7.6 Hz, 2H), 1.90-1.70 (m, 2H), 1.40 (s, 6H).

### Example 2(108)

N-hydroxy-5-[4-(4-chlorophenyl)phenyl]-5-hydroxypentanamide 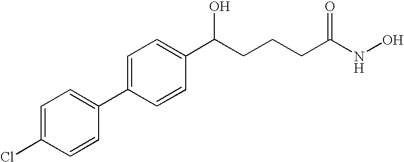
TLC: Rf 0.40 (ethyl acetate:methanol=9:1);
NMR (CDCl₃+DMSO-d₆): δ 10.35 (s, 1H), 8.64 (s, 1H), 7.62-7.50 (m, 4H), 7.45-7.37 (m, 4H), 5.11 (d, J=4.2 Hz, 1H), 4.60 (m, 1H), 2.03 (t, J=6.6 Hz, 2H), 1.80-1.50 (m, 4H).

### Example 2(109)

N-hydroxy-6-hydroxy-6-(4-iodophenyl)hexanamide 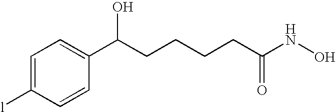
TLC: Rf 0.21 (ethyl acetate);
NMR (DMSO-d₆): δ 10.27 (s, 1H), 8.62 (s, 1H), 7.64 (d, J=8.4 Hz, 2H), 7.10 (d, J=8.4 Hz, 2H), 5.16 (d, J=4.2 Hz, 1H), 4.46-4.41 (m, 1H), 1.88 (t, J=7.2 Hz, 2H), 1.56-1.09 (m, 6H).

### Example 2(110)

N-hydroxy-6-hydroxy-6-(naphthalen-2-yl)hexanamide 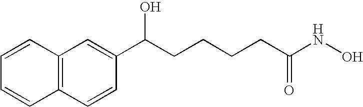
TLC: Rf 0.29 (ethyl acetate);
NMR (DMSO-d₆): δ 10.27 (s, 1H), 8.61 (s, 1H), 7.87-7.82 (m, 3H), 7.78 (s, 1H), 7.49-7.41 (m, 3H), 5.23 (d, J=4.5 Hz, 1H), 4.67-4.62 (m, 1H), 1.89 (t, J=7.5 Hz, 2H), 1.70-1.58 (m, 2H), 1.53-1.43 (m, 2H), 1.39-1.13 (m, 2H).

### Example 2(111)

(7E)-N-hydroxy-6-hydroxy-8-phenyloct-7-enamide 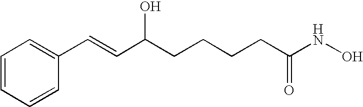
TLC: Rf 0.17 (ethyl acetate);
NMR (DMSO-d₆): δ 10.32 (s, 1H), 8.65 (s, 1H), 7.50-7.15 (m, 5H), 6.50 (d, J=15.8 Hz, 1H), 6.25 (dd, J=15.8 Hz and 5.8 Hz, 1H), 4.82 (d, J=4.8 Hz, 1H), 4.09 (m, 1H), 1.95 (t, J=7.2 Hz, 1H), 1.70-1.20 (m, 6H).

### Example 2(112)

(7E, 9E)-N-hydroxy-6-hydroxy-10-phenyldec-7,9-dienamide 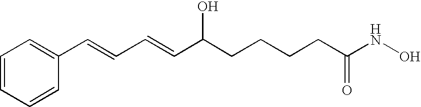
TLC: Rf 0.30 (ethyl acetate:methanol=19:1);
NMR (DMSO-d₆): δ 10.32 (brs, 1H), 8.65 (brs, 1H), 7.45 (m, 1H), 7.32 (m, 2H), 7.21 (m, 1H), 6.88 (dd, J=15.5 Hz and 10.5 Hz, 1H), 6.54 (d, J=15.5 Hz, 1H), 6.32 (dd, J=15.5 Hz and 10.5 Hz, 1H), 5.84 (dd, J=15.5 Hz and 6.3 Hz, 1H), 4.77 (d, J=4.5 Hz, 1H), 4.02 (m, 1H), 1.94 (t, J=7.2 Hz, 2H), 1.60-1.35 (m, 4H), 1.35-1.20 (m, 2H).

### Example 2(113)

N-hydroxy-6-(benzo[b]thiophen-2-yl)-6-hydroxyhexanamide 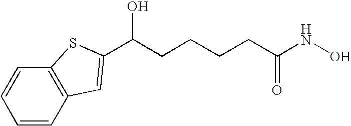
TLC: Rf 0.18 (ethyl acetate);
NMR (DMSO-d₆): δ 10.29 (s, 1H), 8.62 (s, 1H), 7.89-7.86 (m, 1H), 7.75-7.72 (m, 1H), 7.34-7.23 (m, 2H), 7.21 (s, 1H), 5.68 (d, J=4.5 Hz, 1H), 4.85-4.79 (m, 1H), 1.91 (t, J=7.2 Hz, 2H), 1.75-1.67 (m, 2H), 1.55-1.21 (m, 4H).

### Example 2(114)

N-hydroxy-6-(benzo[b]thiophen-3-yl)-6-hydroxyhexanamide 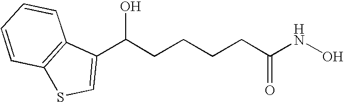
TLC: Rf 0.18 (ethyl acetate);
NMR (DMSO-d₆): δ 10.28 (s, 1H), 8.62 (s, 1H), 7.95-7.88 (m, 2H), 7.49 (s, 1H), 7.38-7.32 (m, 2H), 5.26 (d, J=4.8 Hz, 1H), 4.90-4.86 (m, 1H), 1.90 (t, J=6.9 Hz, 2H), 1.80-1.65 (m, 2H), 1.55-1.20 (m, 4H).

### Example 2(115)

N-hydroxy-6-hydroxy-6-(4-phenoxyphenyl)hexanamide 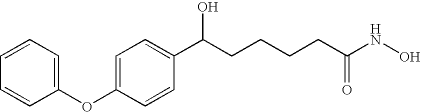
TLC: Rf 0.27 (ethyl acetate);
NMR (DMSO-d₆): δ 10.31 (brs, 1H), 8.56 (brs, 1H), 7.40-7.34 (m, 2H), 7.31 (d, J=8.7 Hz, 2H), 7.14-7.09 (m, 1H), 6.99-6.96 (m, 2H), 6.95 (d, J=8.7 Hz, 2H), 5.10 (d, J=4.2 Hz, 1H), 4.51-4.45 (m, 1H), 1.92 (t, J=7.2 Hz, 2H), 1.65-1.44 (m, 4H), 1.40-1.15 (m, 2H).

### Example 2(116)

N-hydroxy-6-[4-(4-chlorophenyl)phenyl]-6-hydroxyhex-2-ynamide 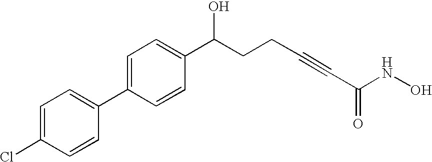
TLC: Rf 0.20 (ethyl acetate);
NMR (DMSO-d₆): δ 10.99 (s, 1H), 9.11 (s, 1H), 7.69 (d, J=8.4 Hz, 2H), 7.63 (d, J=8.1 Hz, 2H), 7.50 (d, J=8.4 Hz, 2H), 7.43 (d, J=8.1 Hz, 2H), 5.40 (d, J=4.8 Hz, 1H), 4.75-4.60 (m, 1H), 2.60-2.20 (m, 2H), 1.90-1.75 (m, 2H).

### Example 2(117)

N-hydroxy-6-hydroxy-6-{4-[2-(pyridin-4-yl)ethyl]phenyl}hexanamide 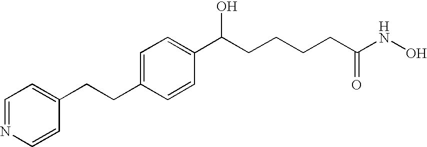
TLC: Rf 0.22 (ethyl acetate:methanol=9:1);
NMR (DMSO-d₆): δ 10.28 (s, 1H), 8.63 (s, 1H), 8.41 (d, J=5.7 Hz, 2H), 7.22 (d, J=5.7 Hz, 2H), 7.18 (d, J=8.4 Hz, 2H), 7.13 (d, J=8.4 Hz, 2H), 5.01 (d, J=4.2 Hz, 1H), 4.44-4.38 (m, 1H), 2.86 (s, 4H), 1.88 (t, J=7.2 Hz, 2H), 1.60-1.09 (m, 6H).

### Example 2(118)

N-hydroxy-6-hydroxy-6-(4-phenethylphenyl)hexanamide 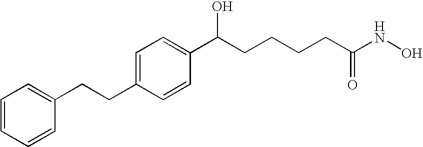
TLC: Rf 0.34 (ethyl acetate);
NMR (DMSO-d₆): δ 10.28 (brs, 1H), 8.62 (brs, 1H), 7.28-7.12 (m, 9H), 5.00 (brs, 1H), 4.45-4.38 (m, 1H), 2.83 (s, 4H), 1.88 (t, J=7.5 Hz, 2H), 1.57-1.40 (m, 4H), 1.17-1.08 (m, 2H).

### Example 2(119)

N-hydroxy-6-[4-(4-chlorophenyl)phenyl]-6-hydroxy-2-methylhexanamide 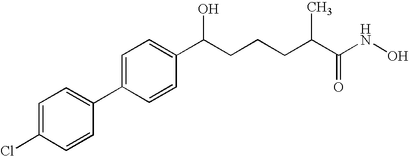
TLC: Rf 0.32 (ethyl acetate);
NMR (DMSO-d₆): δ 10.33 (brs, 1H), 8.65 (brs, 1H), 7.68-7.57 (m, 4H), 7.50-7.30 (m, 4H), 5.14-5.11 (m, 1H), 4.54-4.48 (m, 1H), 2.07-2.00 (m, 1H), 1.62-1.42 (m, 3H), 1.34-1.15 (m, 3H), 0.94 (d, J=6.9 Hz, 3H).

### Example 2(120)

N-hydroxy-6-[4-(4-chlorophenyl)phenyl]-6-hydroxy-2-(pyridin-4-ylmethyl)hexanamide 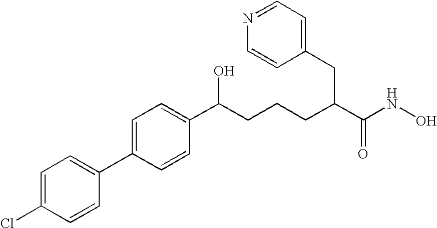
TLC: Rf 0.45 (methylene chloride:ethyl acetate=4:1);
NMR (DMSO-d₆): δ 10.34 (s, 1H), 8.41 (d, J=5.3 Hz, 2H), 7.67 (d, J=8.5 Hz, 2H), 7.59 (d, J=8.5 Hz, 2H), 7.49 (d, J=8.5 Hz, 2H), 7.37 (d, J=8.5 Hz, 1H), 7.36 (d, J=8.5 Hz, 1H), 7.13 (d, J=5.3 Hz, 2H), 5.14 (m, 1H), 4.49 (m, 1H), 2.74 (dd, J=13.8, 9.5 Hz, 1H), 2.58 (dd, J=13.8, 6.0 Hz, 1H), 2.32-2.22 (m, 1H), 1.62-1.46 (m, 3H), 1.40-1.18 (m, 3H).

### Example 2(121)

N-hydroxy-4-{1-[4-(4-chlorophenyl)phenyl]-2-hydroxyethoxy}butanamide 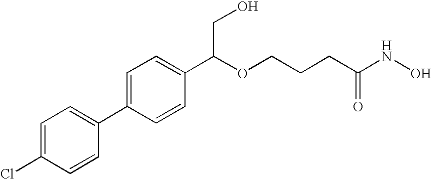
TLC: Rf 0.37 (ethyl acetate:methanol=9:1);
NMR (DMSO-d₆): δ 10.32 (br, 1H), 8.66 (br, 1H), 7.68 (d, J=8.4 Hz, 2H), 7.62 (d, J=8.4 Hz, 2H), 7.50 (d, J=8.4 Hz, 2H), 7.37 (d, J=8.4 Hz, 2H), 4.79 (m, 1H), 4.31 (dd, J=6.9, 4.5 Hz, 1H), 3.54 (dd, J=11.5, 6.9 Hz, 1H), 3.41 (dd, J=11.5, 4.5 Hz, 1H), 3.31-3.27 (m, 2H), 2.09-1.93 (m, 2H), 1.77-1.68 (m, 2H).

### Example 2(122)

N-hydroxy-8-(1,3-dioxan-2-yl)-6-hydroxy-6-(4-phenylphenyl)octanamide 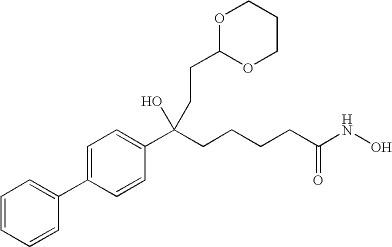
TLC: Rf 0.26 (chloroform:methanol=9:1);
NMR (DMSO-d₆): δ 10.24 (s, 1H), 8.61 (s, 1H), 7.66-7.65 (m, 2H), 7.56 (d, J=8.4 Hz, 2H), 7.46-7.30 (m, 5H), 4.65 (s, 1H), 4.34 (t, J=5.4 Hz, 1H), 3.92-3.87 (m, 2H), 3.63-3.55 (m, 2H), 1.86-1.23 (m, 12H), 1.11-0.82 (m, 2H).

### Example 2(123)

N-hydroxy-4-[1-hydroxy-1-(4-phenylphenyl)methyl]benzamide 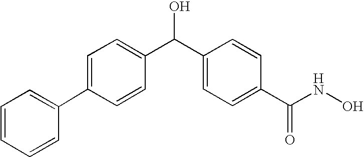
TLC: Rf 0.32 (ethyl acetate);
NMR (DMSO-d₆): δ 11.13 (brs, 1H), 8.98 (brs, 1H), 7.68 (d, J=8.1 Hz, 2H), 7.62-7.57 (m, 4H), 7.48-7.40 (m, 6H), 7.35-7.30 (m, 1H), 6.03 (d, J=3.9 Hz, 1H), 5.78 (d, J=3.9 Hz, 1H).

### Example 2(124)

N-hydroxy-3-{2-[4-(4-chlorophenyl)phenyl]-2-hydroxyethylthio}propanamide 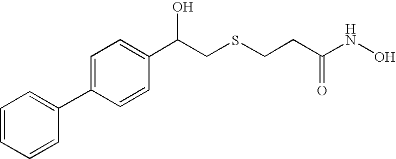
TLC: Rf 0.29 (chloroform:methanol=9:1);
NMR (DMSO-d₆): δ 10.55-10.20 (br, 1H), 8.90-8.65 (br, 1H), 7.68 (d, J=8.8 Hz, 2H), 7.61 (d, J=8.4 Hz, 2H), 7.49 (d, J=8.8 Hz, 2H), 7.44 (d, J=8.4 Hz, 2H), 5.60-5.40 (m, 1H), 4.87-4.62 (m, 1H), 2.80-2.60 (m, 4H), 2.21 (t, J=7.2 Hz, 2H).

### Example 2(125)

N-hydroxy-3-{2-[4-(4-chlorophenyl)phenyl]-2-hydroxyethoxy}benzamide 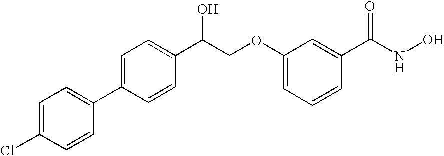
TLC: Rf 0.32 (chloroform:methanol=9:1);
NMR (DMSO-d₆): δ 11.18 (s, 1H), 9.01 (s, 1H), 7.69 (d, J=8.4 Hz, 2H), 7.66 (d, J=8.4 Hz, 2H), 7.55 (d, J=8.4 Hz, 2H), 7.50 (d, J=8.4 Hz, 2H), 7.38-7.22 (m, 3H), 7.15-7.02 (m, 1H), 5.71 (d, J=4.8 Hz, 1H), 4.99 (m, 1H), 4.08 (d, J=5.8 Hz, 2H).

### Example 2(126)

(2E)-N-hydroxy-6-[4-(4-chlorophenyl)phenyl]-6-hydroxyhex-2-enamide 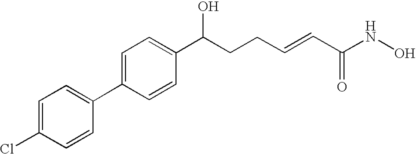
TLC: Rf 0.24 (chloroform:methanol=9:1);
NMR (DMSO-d₆): δ 10.49 (br s, 1H), 7.68 (d, J=8.8 Hz, 2H), 7.61 (d, J=8.0 Hz, 2H), 7.49 (d, J=8.8 Hz, 2H), 7.40 (d, J=8.0 Hz, 2H), 6.66 (dt, J=15.8, 6.6 Hz, 1H), 5.72 (d, J=15.8 Hz, 1H), 4.57 (t, J=6.4 Hz, 1H), 2.30-2.10 (m, 2H), 1.80-1.60 (m, 2H).

### Example 2(127)

N-hydroxy-2-{2-[4-(4-chlorophenyl)phenyl]-2-hydroxyethylthio}acetamide 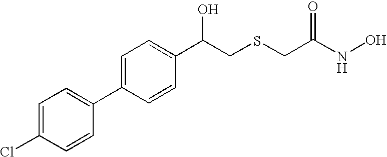
TLC: Rf 0.30 (chloroform:methanol=9:1);
NMR (DMSO-d₆): δ 10.80-10.20 (br, 1H), 9.10-8.80 (br, 1H), 7.68 (d, J=8.4 Hz, 2H), 7.62 (d, J=8.4 Hz, 2H), 7.50 (d, J=8.4 Hz, 2H), 7.44 (d, J=8.4 Hz, 2H), 5.80-5.30 (m, 1H), 4.75 (t, J=6.3 Hz, 1H), 3.03 (s, 2H), 2.87 (d, J=6.3 Hz, 2H).

### Example 2(128)

N-hydroxy-6-[4-(4-chlorophenyl)phenyl]-7-hydroxyheptanamide 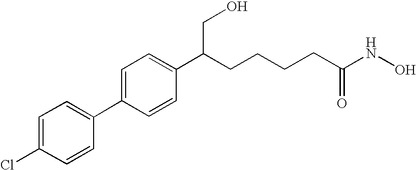
TLC: Rf 0.28 (ethyl acetate:methanol=9:1);
NMR (DMSO-d₆): δ 10.26 (s, 1H), 8.61 (s, 1H), 7.66 (d, J=8.4 Hz, 2H), 7.56 (d, J=8.4 Hz, 2H), 7.48 (d, J=8.4 Hz, 2H), 7.26 (d, J=8.4 Hz, 2H), 4.59 (t, J=5.5 Hz, 1H), 3.49 (t, J=5.5 Hz, 2H), 2.70-2.60 (m, 1H), 1.86 (t, J=7.2 Hz, 2H), 1.81-1.70 (m, 1H), 1.55-1.36 (m, 3H), 1.17-1.03 (m, 2H).

### Example 2(129)

N-hydroxy-4-{2-[4-(4-chlorophenyl)phenyl]-2-hydroxyethoxy}benzamide 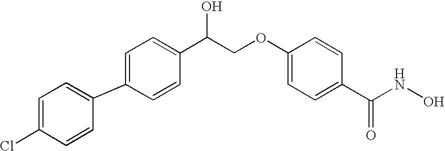
TLC: Rf 0.27 (chloroform:methanol=9:1);
NMR (DMSO-d₆): δ 11.30-10.70 (br, 1H), 9.20-8.60 (br, 1H), 7.80-7.60 (m, 6H), 7.54 (d, J=8.6 Hz, 2H), 7.50 (d, J=8.4 Hz, 2H), 6.99 (d, J=8.8 Hz, 2H), 5.72 (m, 1H), 4.99 (m, 1H), 4.10 (d, J=5.8 Hz, 2H).

### Example 2(130)

(2E)-N-hydroxy-6-[4-(4-chlorophenyl)phenyl]-6-hydroxy-2-methylhex-2-enamide 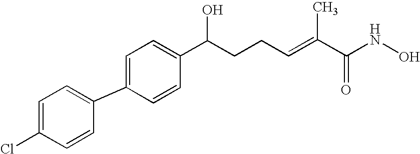
TLC: Rf 0.27 (chloroform:methanol=9:1);
NMR (DMSO-d₆): δ 10.62-10.42 (br, 1H), 7.68 (d, J=8.4 Hz, 2H), 7.61 (d, J=8.0 Hz, 2H), 7.49 (d, J=8.4 Hz, 2H), 7.41 (d, J=8.0 Hz, 2H), 6.23-6.09 (m, 1H), 4.57 (t, J=6.2 Hz, 1H), 2.20-2.03 (m, 2H), 1.80-1.57 (m, 5H).

### Example 2(131)

N-hydroxy-6-[4-(4-chlorophenyl)phenyl]-6-hydroxy-6-(4-methoxyphenyl)hexanamide 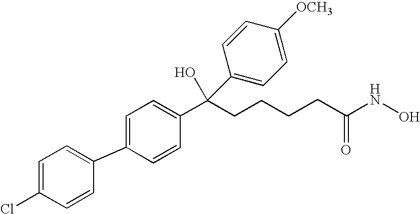
TLC: Rf 0.22 (ethyl acetate);
NMR (DMSO-d₆): δ 10.26 (brs, 1H), 8.63 (brs, 1H), 7.64 (d, J=8.4 Hz, 2H), 7.54 (d, J=8.4 Hz, 2H), 7.47 (d, J=8.4 Hz, 4H), 7.34 (d, J=9.0 Hz, 2H), 6.81 (d, J=9.0 Hz, 2H), 5.40 (s, 1H), 3.69 (s, 1H), 2.19 (t, J=7.5 Hz, 2H), 1.88 (t, J=7.5 Hz, 2H), 1.48 (m, 2H), 1.23-1.13 (m, 2H).

### Example 2(132)

N-hydroxy-6-[4-(4-chlorophenyl)phenyl]-6-hydroxy-9-methoxynon-7-ynamide 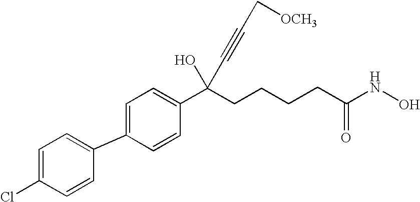
TLC: Rf 0.23 (ethyl acetate);
NMR (DMSO-d₆): δ 10.27 (s, 1H), 8.63 (s, 1H), 7.69 (d, J=8.4 Hz, 2H), 7.64 (d, J=8.4 Hz, 2H), 7.59 (d, J=8.4 Hz, 2H), 7.50 (d, J=8.4 Hz, 2H), 6.05 (s, 1H), 4.18 (s, 2H), 3.32 (s, 3H), 1.90-1.77 (m, 4H), 1.46-1.22 (m, 4H).

### Example 2(133)

(6R)-N-hydroxy-6-hydroxy-6-[4-(3-phenoxyprop-1-ynyl)phenyl]hexanamide 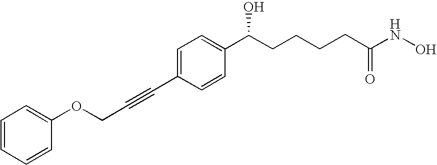
TLC: Rf 0.35 (chloroform:methanol:acetic acid=90:10:1);
NMR (DMSO-d₆): δ 10.27 (brs, 1H), 8.61 (brs, 1H), 7.36 (d, J=8.4 Hz, 2H), 7.28 (d, J=8.4 HZ, 2H), 5.18 (d, J=4.8 Hz, 1H), 5.00 (s, 1H), 4.51-4.42 (m, 1H), 1.84 (t, J=7.2 Hz, 2H), 1.60-1.08 (m, 6H).

### Example 2(134)

### (6R)-N-hydroxy-6-[4-(benzoylamino)phenyl]-6-hydroxyhexanamide

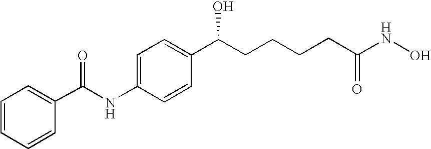 [0709] TLC: Rf 0.30 (chloroform:methanol:acetic acid=6:1:0.1);
NMR (DMSO-d₆): δ 10.30 (s, 1H), 10.15 (s, 1H), 8.62 (s, 1H), 7.91 (dd, J=8.1, 1.5 Hz, 2H), 7.65 (d, J=8.4 Hz, 2H), 7.60-7.42 (m, 3H), 7.23 (d, J=8.4 Hz, 2H), 5.03 (d, J=4.5 Hz, 1H), 4.50-4.35 (m, 1H), 1.87 (t, J=7.2 Hz, 2H), 1.70-1.35 (m, 4H), 1.35-1.00 (m, 2H).

### Example 2(135)

N-hydroxy-6-hydroxy-6-[4-(4-hydroxybut-1-ynyl)phenyl]hexanamide 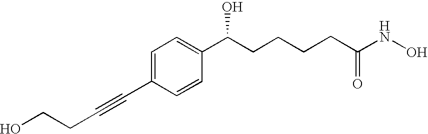
TLC: Rf 0.46 (ethyl acetate:methanol=9:1);
NMR (DMSO-d₆): δ 10.27 (brs, 1H), 8.62 (brs, 1H), 7.30 (d, J=8.4 Hz, 2H), 7.25 (d, J=8.4 Hz, 2H), 5.14 (d, J=4.2 Hz, 1H), 4.86 (t, J=5.4 Hz, 1H), 4.50-4.42 (m, 1H), 3.58-3.52 (m, 2H), 2.54-2.47 (m, 2H), 1.88 (t, J=7.2 Hz, 2H), 1.56-1.08 (m, 6H).

### Example 2(136)

N-hydroxy-6-(4-phenylphenyl)-5,6-dihydroxyhexanamide 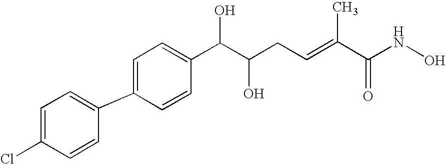
TLC: Rf 0.18 (chloroform:methanol:acetic acid=90:10:1);
NMR (DMSO-d₆): δ 10.25 (s, 1H), 8.61 (s, 1H), 7.64 (d, J=7.2 Hz, 2H), 7.57 (d, J=7.2 Hz, 2H), 7.46-7.30 (m, 5H), 5.17 (d, J=4.2 Hz, 1H), 4.57 (d, J=4.8 Hz, 1H), 4.39-4.36 (m, 1H), 3.57-3.44 (m, 1H), 1.85 (t, J=7.2 Hz, 2H), 1.72-1.59 (m, 1H), 1.49-1.02 (m, 3H).

### Example 2(137)

(6R)-N-hydroxy-6-hydroxy-6-[4-(phenylcarbamoyl)phenyl]hexanamide 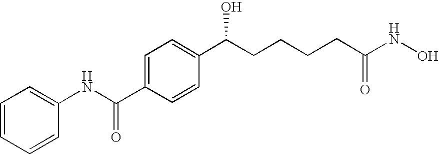
TLC: Rf 0.12 (chloroform:methanol=9:1);
NMR (DMSO-d₆): δ 10.30 (s, 1H), 10.17 (s, 1H), 8.64 (s, 1H), 7.89 (d, J=8.4 Hz, 2H), 7.81-7.73 (m, 2H), 7.44 (d, J=8.4 Hz, 2H), 7.39-7.28 (m, 2H), 7.14-7.02 (m, 1H), 5.26 (d, J=4.4 Hz, 1H), 4.63-4.52 (m, 1H), 1.91 (t, J=7.0 Hz, 2H), 1.68-1.12 (m, 6H).

### Example 2(138)

N-hydroxy-6-[4-(4-chlorophenyl)phenyl]-6-hydroxyiminohexanamide 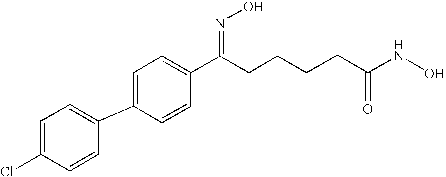
TLC: Rf 0.40 (chloroform:methanol:acetic acid=6:1:0.1);
NMR (DMSO-d₆): δ 11.20 (s, 1H), 10.28 (s,1H), 8.60 (s, 1H), 7.80-7.60 (m, 6H), 7.49 (d, J=8.4 Hz, 2H), 2.80-2.60 (m, 2H), 1.92 (t, J=6.6 Hz, 2H), 1.70-1.25 (m, 4H).

### Example 2(139)

N-hydroxy-6-[4-(4-chlorophenyl)phenyl]heptanamide 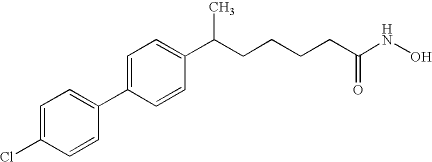
TLC: Rf 0.27 (n-hexane:ethyl acetate=1:2);
NMR (DMSO-d₆): δ 10.27 (brs, 1H), 8.62 (brs, 1H), 7.66 (d, J=8.4 Hz, 2H), 7.57 (d, J=8.4 Hz, 2H), 7.48 (d, J=8.4 Hz, 2H), 7.28 (d, J=8.4 Hz, 2H), 2.73-2.65 (m, 1H), 1.88 (t, J=7.5 Hz, 2H), 1.53 (m, 2H), 1.45 (m, 2H), 1.18 (d, J=6.6 Hz, 3H), 1.18-1.08 (m, 2H).

### Example 2(140)

N-hydroxy-6-[4-(4-chlorophenyl)phenyl]hexanamide 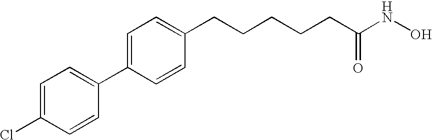
TLC: Rf 0.38 (ethyl acetate);
NMR (DMSO-d₆): δ 10.30 (brs, 1H), 8.64 (brs, 1H), 7.66 (d, J=8.4 Hz, 2H), 7.56 (d, J=8.4 Hz, 2H), 7.48 (d, J=8.4 Hz, 2H), 7.27 (d, J=8.4 Hz, 2H), 2.58 (t, J=7.8 Hz, 2H), 1.92 (t, J=7.3 Hz, 2H), 1.63-1.47 (m, 4H), 1.32-1.23 (m, 2H).

### Example 2(141)

N-hydroxy-3-[(4-phenylbenzyloxycarbonyl)amino]propanamide 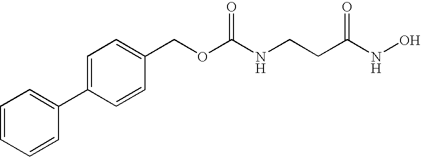
TLC: Rf 0.31 (chloroform:methanol:acetic acid=9:1:0.1);
NMR (DMSO-d₆): δ 10.38 (s, 1H), 8.70 (s, 1H), 7.70-7.50 (m, 4H), 7.50-7.18 (m, 6H), 5.01 (s, 2H), 3.28-3.10 (m, 2H), 2.11 (t, J=7.2 Hz, 2H).

### Example 2(142)

N-hydroxy-2-[(4-phenylbenzyloxycarbonyl)amino]acetamide 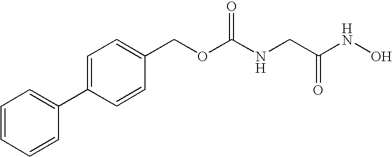
TLC: Rf 0.28 (chloroform:methanol:acetic acid=9:1:0.1);
NMR (DMSO-d₆): δ 10.48 (s, 1H), 8.76 (s, 1H), 7.70-7.50 (m, 4H), 7.50-7.22 (m, 6H), 5.03 (s, 2H), 3.49 (t, J=6.2 Hz, 2H).

### Example 2(143)

(7E)-N-hydroxy-6-methoxy-8-phenloct-7-enamide 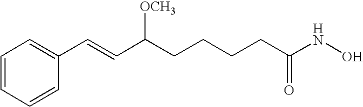
TLC: Rf 0.30 (ethyl acetate);
NMR (DMSO-d₆): δ 10.32 (s, 1H), 8.65 (s, 1H), 7.50-7.20 (m, 5H), 6.56 (d, J=15.8 Hz, 1H), 6.10 (dd, J=15.8 Hz and 7.6 Hz, 1H), 3.69 (m, 1H), 3.20 (s, 3H), 1.94 (t, J=7.2 Hz, 2H), 1.70-1.20 (m, 6H).

### Example 2(144)

N-hydroxy-6-{[4-(benzo[b]furan-2-yl)benzoyl]amino}hexanamide 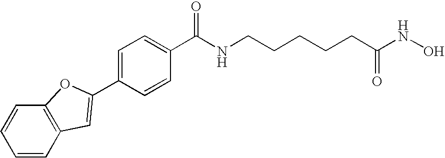
TLC: Rf 0.28 (chloroform:methanol=10:1);
NMR (DMSO-d₆): δ 10.33 (1H, s), 8.80-8.50 (1H, br.s), 8.54 (1H, t, J=5.6 Hz), 8.01 (2H, d, J=8.8 Hz), 7.95 (2H, d, J=8.8H), 7.71-7.62 (2H, m), 7.56 (1H, s), 7.39-7.23 (2H, m), 3.30-3.21 (2H, m), 1.95 (2H, t, J=7.2 Hz), 1.59-1.46 (4H, m), 1.36-1.20 (2H, m).

### Example 2(145)

N-hydroxy-4-[4-(benzo[b]furan-2-yl)benzyloxy]butanamide 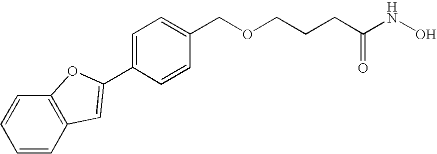
TLC: Rf 0.31 (chloroform:methanol=9:1);
NMR (DMSO-d₆): δ 10.37 (1H, s), 8.68 (1H, s), 7.90 (2H, d, J=8.1 Hz), 7.70-7.58 (2H, m), 7.46 (2H, d, J=8.1 Hz), 7.42 (1H, s), 7.36-7.22 (2H, m), 4.51 (2H, s), 3.46 (2H, t, J=6.3 Hz), 2.06 (2H, t, J=7.4 Hz), 1.87-1.73 (2H, m).

### Example 3 to Example 3(2)

By the same procedures as described in Examples 1 and 2 using corresponding carboxylic acids instead of the compound prepared in Reference Example 1, the compounds of the present invention having the following physical data were obtained.

### Example 3

N-hydroxy-4-{t-butoxycarbonyl[4-(4-chlorophenyl)phenylmethyl]amino}butanamide 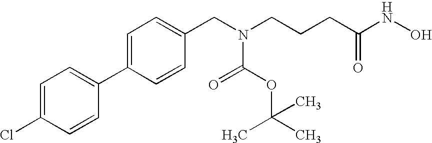
TLC: Rf 0.36 (ethyl acetate);
NMR (CDCl₃): δ 10.22 (br, 1H), 8.80-8.20 (br, 1H), 7.50 (d, J=8.5 Hz, 2H), 7.49 (d, J=8.5 Hz, 2H), 7.39 (d, J=8.5 Hz, 2H), 7.25 (d, J=8.5 Hz, 2H), 4.42 (s, 2H), 3.27 (br, 2H), 2.18 (br, 2H), 1.88-1.79 (m, 2H), 1.46 (s, 9H).

### Example 3(1)

N-hydroxy-5-{t-butoxycarbonyl[4-(4-chlorophenyl)phenylmethyl]amino}pentanamide 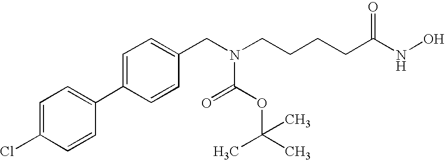
TLC: Rf 0.36 (ethyl acetate);
NMR (CDCl₃): δ 9.55 (br, 1H), 9.00-8.30 (br, 1H), 7.50 (d, J=8.5 Hz, 4H), 7.39 (d, J=8.5 Hz, 2H), 7.26 (d, J=8.5 Hz, 2H), 4.42 (s, 2H), 3.23 (br, 2H), 2.20 (br, 2H), 1.66 -1.50 (m, 4H), 1.45 (s, 9H).

### Example 3(2)

N-hydroxy-6-{t-butoxycarbonyl[4-(4-chlorophenyl)phenylmethyl]amino}hexanamide 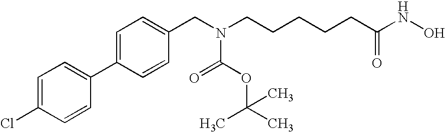
TLC: Rf 0.36 (ethyl acetate);
NMR (CDCl₃): δ 9.66-8.70 (br, 2H), 7.49 (d, J=8.4 Hz, 4H), 7.38 (d, J=8.4 Hz, 2H), 7.26 (d, J=8.4 Hz, 2H), 4.41 (brs, 2H), 3.18 (br, 2H), 2.13 (m, 2H), 1.66-1.44 (m, 4H), 1.44 (brs, 9H), 1.31-1.24 (m, 2H).

### Example 4

N-hydroxy-4-{[4-(4-chlorophenyl)phenylmethyl]amino}butanamide hydrochloride

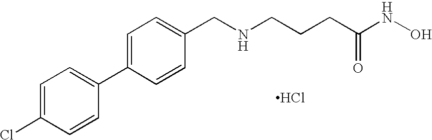

To a solution of the compound prepared in Example 3 (700 mg) in the mixed solution of ethyl acetate, tetrahydrofuran and methylene chloride, 4N hydrochloric acid-ethyl acetate (5 ml) was added at 0° C. and the mixture was stirred for 1 hour at room temperature. The white precipitated solid was filtered, washed with hexane-ethyl acetate to give the compound of the present invention (520 mg) having the following physical data.
TLC: Rf 0.19 (methylene chloride:methanol=4:1);
NMR (CD₃OD): δ 7.71 (d, J=8.7 Hz, 2H), 7.62 (d, J=8.7 Hz, 2H), 7.58 (d, J=8.7 Hz, 2H), 7.45 (d, J=8.7 Hz, 2H), 4.25 (s, 2H), 3.13 (t, J=7.2 Hz, 2H), 2.29 (t, J=7.2 Hz, 2H), 2.00 (quint, J=7.2 Hz, 2H).

### Example 4(1) to Example 4(2)

By the same procedure as described in Example 4 using the compounds prepared in Example 3(1) to Example 3(2) instead of the compound prepared in Example 3, the compounds of the present invention having the following physical data were obtained.

### Example 4(1)

N-hydroxy-5-{[4-(4-chlorophenyl)phenylmethyl]amino}pentanamide hydrochloride 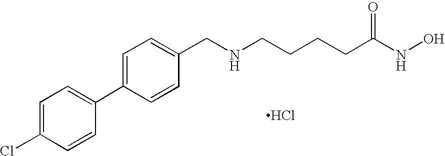
TLC: Rf 0.23 (methylene chloride:methanol=4:1);
NMR (CD₃OD): δ 7.71 (d, J=8.7 Hz, 2H), 7.63 (d, J=8.7 Hz, 2H), 7.59 (d, J=8.7 Hz, 2H), 7.45 (d, J=8.7 Hz, 2H), 4.24 (s, 2H), 3.08 (t, J=7.2 Hz, 2H), 2.19 (t, J=6.7 Hz, 2H), 1.82-1.66 (m, 4H).

### Example 4(2)

N-hydroxy-6-{[4-(4-chlorophenyl)phenylmethyl]amino}hexanamide hydrochloride 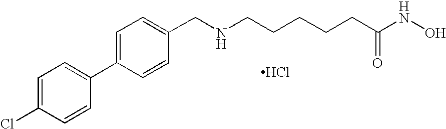
TLC: Rf 0.24 (methylene chloride:methanol=4:1);
NMR (CD₃OD): δ 7.72 (d, J=8.7 Hz, 2H), 7.63 (d, J=8.7 Hz, 2H), 7.58 (d, J=8.7 Hz, 2H), 7.45 (d, J=8.7 Hz, 2H), 4.24 (s, 2H), 3.06 (t, J=7.8 Hz, 2H), 2.15 (t, J=7.2 Hz, 2H), 1.74 (m, 2H), 1.66 (m, 2H), 1.42 (m, 2H).

### Reference Example 2

5-[4-(4-methylphenyl)benzoyl]pentanoic acid methyl ester

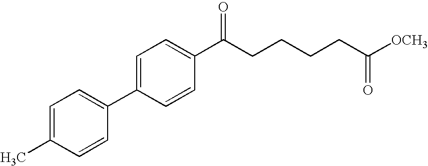

To a suspension of aluminium trichloride (14 g) in a methylene chloride (400 ml), 4-(4-methylphenyl)benzene (10.0 g) was added at 0° C., then a solution of methyl adipoyl chloride (8.2 ml) in methylene chloride (40 ml) was dropped thereto. The mixture was stirred for 5 hours at 0° C., then reaction temperature was raised up to room temperature. The reaction mixture was poured into iced water carefully and extracted with methylene chloride. The organic layer was washer with 1N hydrochloric acid and water sequentially, dried over anhydrous magnesium sulfate and concentrated to give the title compound (17.4 g) having the following physical data.
TLC: Rf 0.63 (n-hexane:ethyl acetate=3:1).

### Reference Example 3

6-hydroxy-6-[4-(4-methylphenyl)phenyl]hexanoic acid methyl ester

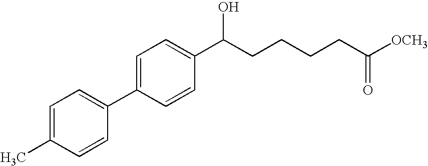

To a solution of the compound prepared in Reference Example 2 (15.4 g) in methylene chloride (125 ml)—methanol (125 ml), sodium borohydride (1.47 g) was added at 0° C. and the mixture was stirred for 2 hours at same temperature. The reaction mixture was added by saturated aqueous solution of ammonium chloride and extracted with chloroform. The organic layer was washed with water and brine sequentially, dried anhydrous sodium sulfate and concentrated under reduced pressure. The residue was purified by column chromatography on silica gel (n-hexane:ethyl acetate=from 85:15 to 75:25) to give the title compound (14.2 g) having the following physical data.
TLC: Rf 0.35 (n-hexane:ethyl acetate=2:1);

### Reference Example 4

6-(t-butyldimethylsilyloxy)-6-[4-(4-methylphenyl)phenyl]hexanoic acid methyl ester

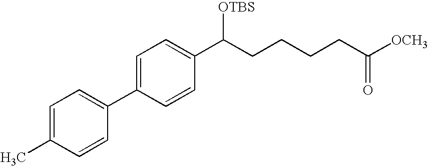

To a solution of the compound prepared in Reference Example 3 (3.59 g) in N,N-dimethylformamide (22 ml), imidazole (2.11 g) and t-butyldimethylsilylchloride (2.60 g) was added sequentially and the mixture was stirred over night at room temperature. To the reaction mixture, saturated aqueous solution of ammonium chloride was added, then the mixture was washed with water and brine sequentially, dried over anhydrous sodium sulfate and concentrated under reduced pressure. The residue was purified by column chromatography on silica gel (n-hexane:ethyl acetate=95:5) to give the title compound (4.92 g) having the following physical data.
TLC: Rf 0.62 (n-hexane:ethyl acetate=8:1);
NMR (CDCl₃): δ 7.51 (d, J=8.4 Hz, 2H), 7.49 (d, J=8.4 Hz, 2H), 7.31 (d, J=8.4 Hz, 2H), 7.23 (d, J=8.4 Hz, 2H), 4.69-4.65 (m, 1H), 3.64 (s, 3H), 2.38 (s, 3H), 2.28 (t, J=7.2 Hz, 2H), 1.81-1.21 (m, 6H), 0.89 (s, 9H), 0.03 (s, 3H), −0.12 (s, 3H).

### Reference Example 5

6-(t-butyldimethylsilyloxy)-6-[4-(4-methylphenyl)phenyl]hexanol

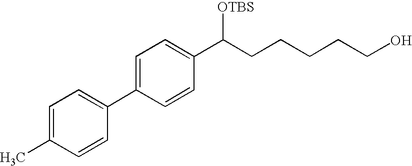

To a solution of lithium aluminium hydride (437 mg) in tetrahydrofuran (50 ml), the compound prepared in Reference Example 4 (4.90 g) in tetrahydrofuran (50 ml) was added dropwise at 0° C. and the mixture was stirred for 3 hours at room temperature. The mixture was cooled to 0° C. again, then aqueous solution of sodium sulfate was added to the reaction mixture. The precipitate was removed to give the title compound (4.88 g) having the following physical data. This crude compound was used in the next reaction without further purification.
TLC: Rf 0.53 (n-hexane:ethyl acetate=2:1);
NMR (CDCl₃): δ 7.52 (d, J=8.4 Hz, 2H), 7.50 (d, J=8.4 Hz, 2H), 7.33 (d, J=8.4 Hz, 2H), 7.23 (d, J=8.4 Hz, 2H), 4.69-4.65 (m, 1H), 3.76-3.72 (m, 1H), 3.61 (t, J=6.6 Hz, 2H), 2.38 (s, 3H), 1.86-1.21 (m, 8H), 0.89 (s, 3H), 0.04 (s, 3H), −0.11 (s, 3H).

### Reference Example 6

4-methylbenzenesulfonic acid 6-(t-butyldimethylsilyloxy)-6-[4-(4-methylphenyl)phenyl]hexyl ester

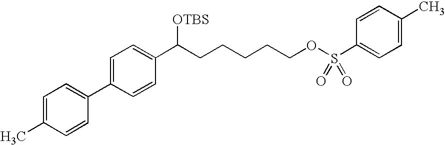

To a solution of the compound prepared in Reference Example 5 (3.08 g) in methylene chloride (39 ml), pyridine (5.31 ml) and p-toluenesulfonyl chloride (1.77 g) was added at 0° C., then the reaction temperature was raised up to room temperature and the mixture was stirred over night. The reaction mixture was concentrated, then the residue was added by the saturated aqueous solution of ammonium chloride and extracted with ethyl acetate. The organic layer was washed with water, saturated aqueous solution of sodium hydrogen carbonate and brine sequentially, dried over anhydrous sodium sulfate and concentrated under reduced pressure. The residue was purified by column chromatography on silica gel (n-hexane:ethyl acetate=95:5) to give the title compound (2.71 g) having the following physical data.
TLC: Rf 0.53 (n-hexane:ethyl acetate=6:1);
NMR (CDCl₃): δ 7.76 (d, J=8.4 Hz, 2H), 7.52-7.48 (m, 4H), 7.33-7.21 (m, 6H), 4.64-4.60 (m, 1H), 3.99 (t, J=7.2 Hz, 2H), 2.43 (s, 3H), 2.38 (s, 3H), 1.71-1.21 (m, 8H), 0.88 (s, 9H), 0.11 (s, 3H), −0.13 (s, 3H).

### Reference Example 7

6-t-butyldimethylsilyloxy-6-[4-(4-methylphenyl)phenyl]hexylphosphonic acid diethyl ester

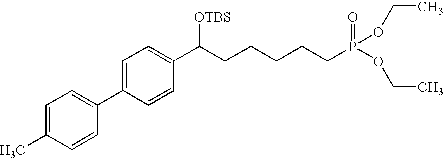

To a suspension of sodium hydride (304 mg) in N,N-dimethylformamide (60 ml), diethylphosphonic acid (1.05 g) was added and the mixture was stirred for 30 minutes at room temperature. The mixture was added by a solution of the compound prepared in Reference Example 6 (2.10 g) in N,N-dimethylformamide (60 ml), then the mixture was stirred for 2 hours at 85° C. The reaction mixture was added by the saturated aqueous solution of ammonium chloride and extracted with ethyl acetate. The organic layer was washed with water and brine sequentially, dried over anhydrous sodium sulfate, and concentrated under reduced pressure. The residue was purified by column chromatography on silica gel (n-hexane:ethyl acetate=1:1) to give the title compound (1.22 g) having the following physical data.
TLC: Rf 0.35 (n-hexane:ethyl acetate=1:1);
NMR (CDCl₃): δ 7.51 (d, J=8.1 Hz, 2H), 7.50 (d, J=8.1 Hz, 2H), 7.31 (d, J=8.1 Hz, 2H), 7.23 (d, J=8.1 Hz, 2H), 4.67-4.63 (m, 1H), 4.18-3.98 (m, 4H), 2.38 (s, 3H), 1.78-1.23 (m, 10H), 1.30 (t, J=7.2 Hz, 6H), 0.89 (s, 9H), 0.03 (s, 3H), 0.12 (s, 3H).

### Example 5

6-hydroxy-6-[4-(4-methylphenyl)phenyl]hexylphosphonic acid diethyl ester

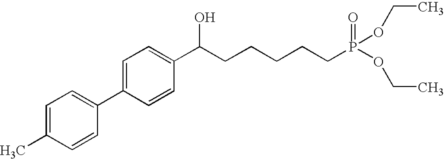

To a solution of the compound prepared in Reference Example 7 (1.27 g) in tetrahydrofuran (24 ml), 1M tetrabutylammonium fluoride (4.9 ml) was added and the mixture was stirred at room temperature. The reaction mixture was added by the saturated aqueous solution of ammonium chloride and extracted with ethyl acetate. The organic layer was washed with 1N hydrochloric acid, saturated aqueous solution of sodium hydrogen carbonate and brine sequentially, dried over anhydrous sodium sulfate and concentrated under reduced pressure. The residue was purified by column chromatography on silica gel (n-hexane:ethyl acetate=1:1, then ethyl acetate only, then ethyl acetate:methanol=9:1) to give the compound in the present invention (1.00 g) having the following physical data.
TLC: Rf 0.12 (n-hexane:ethyl acetate=1:4).

### Example 6

6-hydroxy-6-[4-(4-methylphenyl)phenyl]hexylphosphonic acid

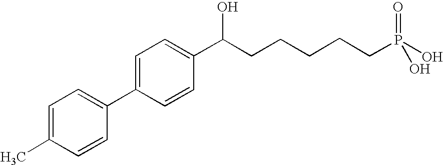

To a solution of the compound prepared in Example 5 (50 mg) in acetonitrile (5 ml), sodium iodide (93 mg) and trimethylsilyl chloride (156 μl) were added sequentially and the mixture was stirred over night at room temperature. The reaction mixture was added by ammonium chloride and extracted with ethyl acetate. The organic layer was washed with saturated aqueous solution of sodium hydrogen carbonate, water and brine sequentially, dried over anhydrous sodium sulfate, concentrated under reduced pressure. The residue was added by tetrahydrofuran (2 ml) and saturated aqueous solution of sodium hydrogen carbonate (2 ml) and refluxed for 2 hours. The reaction mixture was cooled and extracted with ethyl acetate. The organic layer was washed with brine, dried over anhydrous sodium sulfate, concentrated under reduced pressure. The residue was added by diisopropyl ether, filtrated to give the compound in the present invention (29 mg) having the following physical data.
TLC: Rf 0.12 (chloroform:methanol:water=65:25:4);
NMR (DMSO-d₆): δ 7.56 (d, J=8.4 Hz, 2H), 7.53 (d, J=8.4 Hz, 2H), 7.36 (d, J=8.4 Hz, 2H), 7.24 (d, J=8.4 Hz, 2H), 4.54-4.49 (m, 1H), 2.32 (s, 3H), 1.65-1.18 (m, 10H).

### Reference Example 8

5-[4-(4-chlorophenyl)phenyl]-5-hydroxypentanoic acid ethyl ester

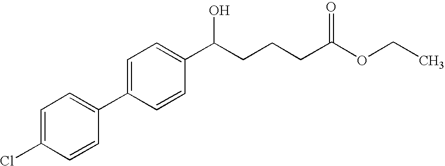

By the same procedures as described in Reference Examples 2 and 3 using 4-chlorobiphenyl instead of 4-(4-methylphenyl)benzene and ethyl glutaryl chloride instead of methyl adipoyl chloride, the compound of the present invention having the following physical data was obtained.
TLC: Rf 0.22 (n-hexane:ethyl acetate=2:1);
NMR (CDCl₃): δ 7.53 (d, J=8.5 Hz, 2H), 7.50 (d, J=8.5 Hz, 2H), 7.41 (d, J=8.5 Hz, 2H), 7.39 (d, J=8.5 Hz, 2H), 4.76-4.71 (m, 1H), 4.12 (q, J=7.2 Hz, 2H), 2.35 (t, J=6.6 Hz, 2H), 2.10 (s, 1H), 1.90-1.60 (m, 4H), 1.24 (t, J=7.2 Hz, 3H).

### Reference Example 9

5-[4-(4-chlorophenyl)phenyl]-5-methoxymethoxypentanoic acid ethyl ester

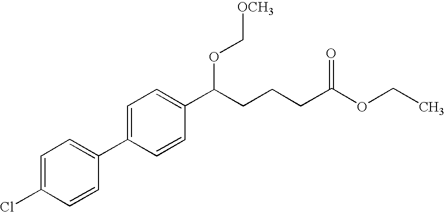

To a solution of the compound prepared in Reference Example 8 (6.0 g) in methylene chloride (100 ml), diisopropylethylamine (21 ml) was added and the mixture was cooled at 0° C., then methoxymethylchloride (6.8 ml) was added dropwise thereto and the mixture was stirred over night at room temperature. The reaction mixture was extracted with methylene chloride. The organic layer was washed with saturated aqueous solution of sodium hydrogen carbonate and water sequentially, dried over anhydrous magnesium sulfate, concentrated to give the title compound (8.5 g) having the following physical data. This crude compound was used in the next reaction without further purification.
TLC: Rf 0.44 (n-hexane:ethyl acetate=2:1);
NMR (CDCl₃): δ 7.52 (d, J=8.7 Hz, 2H), 7.51 (d, J=8.7 Hz, 2H), 7.39 (d, J=8.7 Hz, 2H), 7.36 (d, J=8.7 Hz, 2H), 4.62 (dd, J=7.2, 5.2 Hz, 1H), 4.55 (s, 2H), 4.11 (q, J=7.2 Hz, 2H), 3.38 (s, 3H), 2.33 (t, J=7.2 Hz, 2H), 1.93-1.60 (m, 4H), 1.24 (t, J=7.2 Hz, 3H).

### Reference Example 10

5-[4-(4-chlorophenyl)phenyl]-5-methoxymethoxypentyl-4-methylbenzenesulfonate

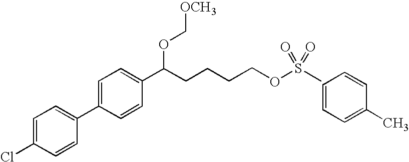

By the same procedures as described in Reference Examples 5 and 6 using the compound prepared in Reference Example 9 instead of the compound prepared in Reference Example 4, the title compound having the following physical data were obtained.
TLC: Rf 0.35 (n-hexane:ethyl acetate=2:1);
NMR (CDCl₃): δ 7.76 (d, J=8.5 Hz, 2H), 7.51 (d, J=8.5 Hz, 2H), 7.51 (d, J=8.5 Hz, 2H), 7.40 (d, J=8.5 Hz, 2H), 7.32 (d, J=8.5 Hz, 4H), 4.55 (dd, J=7.8, 5.4 Hz, 1H), 4.52 (s, 2H), 4.01 (t, J=6.5 Hz, 2H), 3.35 (s, 3H), 2.43 (s, 3H), 1.88-1.32 (m, 6H).

### Example 7

5-acetylthio-1-[4-(4-chlorophenyl)phenyl]-1-methoxymethoxypentane

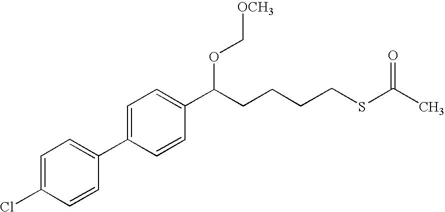

To a solution of the compound prepared in Reference Example 10 (510 mg) in N,N-dimethylformamide (10 ml), potassium thioacetate (140 mg) was added and the mixture was stirred for 2 days at room temperature. The reaction mixture was extracted with ethyl acetate. The organic layer was washed with water and brine sequentially, dried over anhydrous magnesium sulfate, concentrated under reduced pressure. The residue was purified by column chromatography on siliac gel (n-hexane:ethyl acetate=3:1) to give the compound in the present invention (390 mg) having the following physical data.
TLC: Rf 0.38 (n-hexane:ethyl acetate=4:1);
NMR (CDCl₃): δ 7.52 (d, J=8.4 Hz, 2H), 7.51 (d, J=8.4 Hz, 2H), 7.39 (d, J=8.4 Hz, 2H), 7.35 (d, J=8.4 Hz, 2H), 4.59 (dd, J=7.8, 5.2 Hz, 1H), 4.54 (s, 2H), 3.38 (s, 3H), 2.85 (t, J=7.0 Hz, 2H), 2.31 (s, 3H), 1.97-1.35 (m, 4H).

### Example 8

5-acetylthio-1-[4-(4-chlorophenyl)phenyl]pentanol

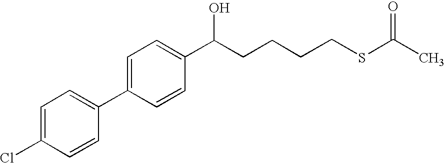

To a solution of the compound prepared in Example 7 (390 mg) in dioxane (10 ml), 1N hydrochloric acid (3 ml) was added and the mixture was stirred 30 minutes at 90° C. The reaction mixture was cooled and extracted with ethyl acetate. The organic layer was washed with water and brine sequentially, dried over anhydrous magnesium sulfate, concentrated under reduced pressure. The residue was purified by column chromatography on silica gel (n-hexane:ethyl acetate=2:1) to give the compound in the present invention (220 mg) having the following physical data.
TLC: Rf 0.30 (n-hexane:ethyl acetate=2:1);
NMR (CDCl₃): δ 7.53 (d, J=8.4 Hz, 2H), 7.51 (d, J=8.4 Hz, 2H), 7.40 (d, J=8.4 Hz, 2H), 7.39 (d, J=8.4 Hz, 2H), 4.72 (t, J=6.5 Hz, 1H), 2.86 (t, J=7.0 Hz, 2H), 2.31 (s, 3H), 1.91-1.35 (m, 6H).

### Example 9

1-[4-(4-chlorophenyl)phenyl]-5-mercaptopentanol

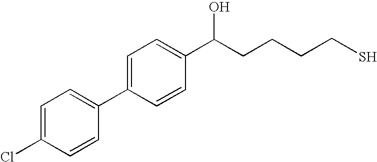

To a solution of the compound prepared in Example 8 (200 mg) in anhydrous methanol (10 ml), 1N aqueous solution of sodium hydroxide (1 ml) was added and the mixture was stirred for 30 minutes as room temperature under atmosphere of argon. The reaction mixture was poured into cold dilute hydrochloric acid. The mixture was extracted with ethyl acetate. The organic layer was washed with water, dried over anhydrous magnesium sulfate, concentrated under reduced pressure. The residue was purified by column chromatography on silica gel (n-hexane:ethyl acetate=2:1) to give the compound in the present invention (150 mg) having the following physical data.
TLC: Rf 0.32 (n-hexane:ethyl acetate=2:1);
NMR (CDCl₃): δ 7.54 (d, J=8.7 Hz, 2H), 7.51 (d, J=8.7 Hz, 2H), 7.41 (d, J=8.7 Hz, 2H), 7.40 (d, J=8.7 Hz, 2H), 4.73 (t, J=6.5 Hz, 1H), 2.53 (q, J=7.5 Hz, 2H), 1.91-1.37 (m, 6H), 1.33 (t, J=7.5 Hz, 1H).

### Example 10

N-amino-6-[4-(4-chlorophenyl)phenyl]-6-hydroxyhexanamide

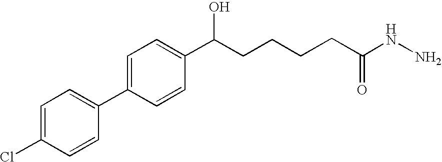

To a solution of 6-hydroxy-6-[4-(4-chlorophenyl)phenyl]hexanoic acid (300 mg) in ethanol (10 ml), hydrazine (5ml) was added and the mixture was stirred over night at room temperature. The precipitate was filtrated, washed with ethanol to give the compound in the present invention (190 mg) having the following physical data.
TLC: Rf 0.38 (methylene chloride:ethyl acetate=6:1);
NMR (DMSO-d₆): δ 8.98 (brs, 1H), 7.68 (d, J=8.5 Hz, 2H), 7.60 (d, J=8.5 Hz, 2H), 7.50 (d, J=8.5 Hz, 2H), 7.39 (d, J=8.5 Hz, 2H), 5.15 (d, J=4.5 Hz, 1H), 4.53 (m, 1H), 4.11 (m, 2H), 1.98 (t, J=7.2 Hz, 2H), 1.65-1.55 (m, 2H), 1.55-1.45 (m, 2H), 1.40-1.19 (m, 2H).

### Example 11

6-[4-(4-chlorophenyl)phenyl]-6-hydroxyhexanamide

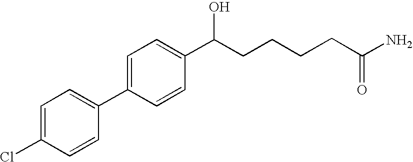

To a solution of 6-hydroxy-6-[4-(4-chlorophenyl)phenyl]hexanoic acid (600 mg) in ethanol (20 ml), aqueous ammonia (5 ml) was added and the mixture was stirred over night at room temperature, then stirred for 5 hours at 70° C. Then gaseous ammonia was blown thereto, saturated and stirred overnight at room temperature. The reaction mixture was extracted with ethyl acetate. The organic layer was washed with 1N hydrochloric acid, water and brine sequentially, dried over anhydrous magnesium sulfate, concentrated under reduced pressure. The residue was purified by column chromatography on silica gel (n-hexane:ethyl acetate=1:2) to give the compound in the present invention (160 mg) having the following physical data.
TLC: Rf 0.20 (ethyl acetate);
NMR (DMSO-d₆): δ 7.68 (d, J=8.5 Hz, 2H), 7.60 (d, J=8.5 Hz, 2H), 7.50 (d, J=8.5 Hz, 2H), 7.40 (d, J=8.5 Hz, 2H), 7.19 (br, 1H),6.65 (br, 1H), 5.15 (d, J=4.5 Hz, 1H), 4.54 (m, 1H), 2.01 (t, J=7.5 Hz, 2H), 1.66-1.56 (m, 2H), 1.54-1.44 (m, 2H), 1.40-1.20 (m, 2H).

### Reference Example 11

6-hydroxy-6-[4-(4-methylphenyl)phenyl]hexanoic acid

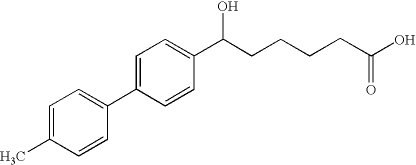

To a solution of the compound prepared in Reference Example 3 (1.25 g) in methanol (300 ml), 5N aqueous solution of sodium hydroxide (6.0 ml) was added and the mixture was stirred for 2 hours at 70° C. The precipitate was removed and filtrate was concentrated under reduced pressure. The residue was extracted with ethyl acetate. The organic layer was washed with 1N hydrochloric acid, water and brine sequentially, dried over anhydrous magnesium sulfate, concentrated to give the title compound (1.11 g) having the following physical data.
TLC: Rf 0.38 (chloroform:methanol:acetic acid=90:10:1);
NMR (DMSO-d₆): δ 11.93 (s, 1H), 7.56 (d, J=8.4 Hz, 2H), 7.53 (d, J=8.4 Hz, 2H), 7.35 (d, J=8.4 Hz, 2H), 7.24 (d, J=8.4 Hz, 2H), 5.12 (brs, 1H), 4.55-4.49 (m, 1H), 2.32 (s, 3H), 2.17 (t, J=7.5 Hz, 2H), 1.64-1.20 (m, 6H).

### Example 12

N-methoxy-N-methyl-6-[4-(4-methylphenyl)phenyl]-6-hydroxyhexanamide

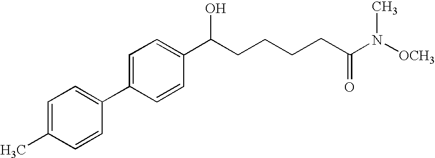

By the same procedure as described in Examples 1 using N-methyl-N-methoxyamine instead of N-(1-methyl-1-methoxyethoxy)amine and the compound prepared in Reference Example 11 instead of the compound prepared in Reference Example 1, the compound of the present invention having the following physical data was obtained.
TLC: Rf 0.41 (chloroform:methanol=95:5);
NMR (CDCl₃): δ 7.55 (d, J=8.4 Hz, 2H), 7.48 (d, J=8.4 Hz, 2H), 7.40 (d, J=8.4 Hz, 2H), 7.24 (d, J=8.4 Hz, 2H), 4.75-4.71 (m, 1H), 3.65 (s, 3H), 3.16 (s, 3H), 2.42 (t, J=7.5 Hz, 2H), 2.39 (s, 3H), 1.91-1.31 (m, 6H).

### Reference Example 12

N-methoxy-N-methyl-6-(t-butyldimethylsilyloxy)-6-[4-(4-methylphenyl)phenyl]hexanamide

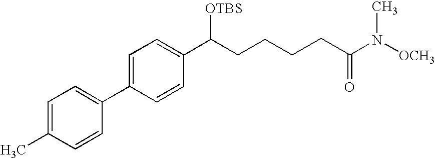

By the same procedure as described in Reference Example 4 using the compound prepared in Example 12 instead of the compound prepared in Reference Example 3, the title compound having the following physical data was obtained.
TLC: Rf 0.49 (n-hexane:ethyl acetate=95:5);
NMR (CDCl₃): δ 7.51 (d, J=8.1 Hz, 2H), 7.50 (d, J=8.1 Hz, 2H), 7.33 (d, J=8.1 Hz, 2H), 7.23 (d, J=8.1 Hz, 2H), 4.70-4.66 (m, 1H), 3.65 (s, 3H), 3.16 (s, 3H), 2.42-2.32 (m, 2H), 2.38 (s, 3H), 1.83-1.25 (m, 6H), 1.89 (s, 9H), 0.33 (s, 3H), −0.11 (s, 3H).

### Reference Example 13

8-(t-butyldimethylsilyloxy)-8-[4-(4-methylphenyl)phenyl]-3-oxooct-1-ene

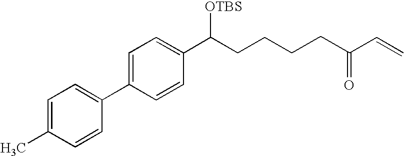

To a solution of the compound prepared in Reference Example 12 (5.46 g) in tetrahydrofuran (60 ml), vinyl magnesium bromide (16.5 ml) was added and the mixture was stirred for 1 hour at −15° C., then stirred for 1 hour at 0° C. The reaction mixture was added by the saturated aqueous solution of ammonium chloride, extracted with ethyl acetate. The organic layer was washed with water and brine sequentially, dried over anhydrous sodium sulfate, concentrated under reduced pressure. The residue was purified by column chromatography on silica gel (n-hexane:ethyl acetate=95:5) to give the title compound having the following physical data.
TLC: Rf 0.53 (n-hexane:ethyl acetate=95:5);
NMR (CDCl₃): δ 7.52 (d, J=8.1 Hz, 2H), 7.50 (d, J=8.1 Hz, 2H), 7.32 (d, J=8.1 Hz, 2H), 7.23 (d, J=8.1 Hz, 2H), 6.33 (dd, J=17.7, 10.2 Hz, 1H), 6.19 (dd, J=17.7, 1.5 Hz, 1H), 5.80 (dd, J=10.2, 1.5 Hz, 1H), 4.69-4.65 (m, 1H), 2.55 (t, J=7.8 Hz,2H), 2.38 (s, 3H), 1.79-1.25 (m, 6H), 0.89 (s, 9H), 0.30 (s, 3H), 0.1.2 (s, 3H).

### Reference Example 14

6-(t-butyldimethylsilyloxy)-6-[4-(4-methylphenyl)phenyl]-1-(oxiran-2-yl)hexan-1-one

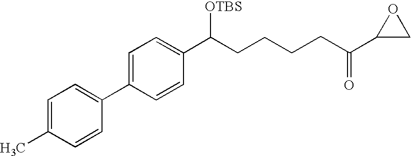

To a solution of the compound prepared in Reference Example 13 (1.32 g) in tetrahydrofuran (30 ml), N-benzyltrimethylammonium hydroxide (Triton B; registered trademark, 0.15 ml) and t-butyl peroxide (3.1 ml) were added at 0° C. and the mixture was stirred for 30 minutes at 0° C. The reaction mixture was added by the saturated aqueous solution of ammonium chloride and water and extracted with ethyl acetate. The organic layer was washed with brine, dried over anhydrous sodium sulfate, concentrated under reduced pressure. The residue was purified by column chromatography on silica gel (n-hexane:ethyl acetate=from 95:5 to 90:10) to give the title compound (1.45 g) having the following physical data.
TLC: Rf 0.32 (n-hexane:ethyl acetate=9:1);
NMR (CDCl₃): δ 7.51 (d, J=8.1 Hz, 2H), 7.49 (d, J=8.1 Hz, 2H), 7.31 (d, J=8.1 Hz, 2H), 7.23 (d, J=8.1 Hz, 2H), 4.68-4.64 (m, 1H), 3.40 (d, J=4.5 Hz, 2.4 Hz, 1H), 2.96 (d, J=6.0, 4.5 Hz, 1H), 2.82 (d, J=6.0 Hz, 2.4 Hz, 1H), 2.38 (s, 3H), 1.80-1.23 (m, 6H), 0.88 (s, 9H), 0.02 (s, 3H), −0.12 (s, 3H).

### Example 13

6-hydroxy-6-[4-(4-methylphenyl)phenyl]-1-(oxiran-2-yl)hexan-1-one

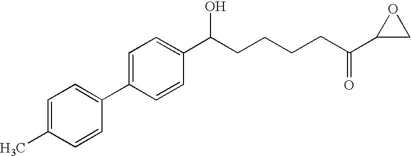

By the same procedure as described in Example 5 using the compound prepared in Reference Example 14 instead of the compound prepared in Reference Example 7, the compound of the present invention having the following physical data was obtained.
TLC: Rf 0.38 (n-hexane:ethyl acetate=1:1);
NMR (DMSO-d₆): δ 7.55 (d, J=8.4 Hz, 2H), 7.53 (d, J=8.4 Hz, 2H), 7.35 (d, J=8.4 Hz, 2H), 7.24 (d, J=8.4 Hz, 2H), 5.11 (d, J=4.5 Hz, 1H), 4.53-4.47 (m, 1H), 3.47-3.44 (m, 1H), 2.95-2.91 (m, 1H), 2.87-2.84 (m, 1H), 2.35 (t, J=6.3 Hz, 2H), 2.31 (s, 3H), 1.63-1.12 (m, 6H).

### Reference Example 15

5-[4-(4-chlorophenyl)benzoyl]pentanoic acid

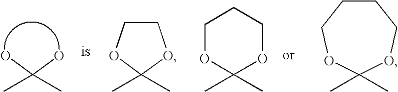

To a solution of 5-[4-(4-chlorophenyl)benzoyl]pentanoic acid ethyl ester (1.25 g) in methanol (300 ml), 5N aqueous solution of sodium hydroxide (6.0 ml) was added and the mixture was stirred for 2 hours at 70° C. The insoluble materials was removed by filtration and filtrate was concentrated under reduced pressure. The residue was extracted with ethyl acetate. The organic layer was washed with 1N hydrochloric acid, water and brine sequentially, dried over anhydrous magnesium sulfate, concentrated to give the title compound (1.11 g) having the following physical data.
TLC: Rf 0.35 (n-hexane:ethyl acetate=1:2);
NMR (CDCl₃): δ 8.02 (d, J=8.4 Hz, 2H), 7.64 (d, J=8.4 Hz, 2H), 7.55 (d, J=8.8 Hz, 2H), 7.43 (d, J=8.8 Hz, 2H), 3.03 (t, J=7.0 Hz, 2H), 2.43 (t, J=7.0 Hz, 2H), 1.90-1.70 (m, 4H).

### Example 14

N-(1-methyl-1-methoxyethoxy)-5-[4-(4-chlorophenyl)benzoyl]pentanamide

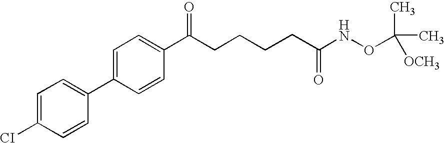

By the same procedure as described in Example 1 using the compound prepared in Reference Example 15 instead of the compound prepared in Reference Example 1, the compound of the present invention having the following physical data was obtained.
TLC: Rf 0.18 (n-hexane:ethyl acetate=1:2);
NMR (CDCl₃): δ 8.02 (d, J=8.7 Hz, 2H), 7.64 (d, J=8.7 Hz, 2H), 7.55 (d, J=8.7 Hz, 2H), 7.44 (d, J=8.7 Hz, 2H), 3.35 (brs, 3H), 3.04 (t, J=6.7 Hz, 2H), 2.50-2.19 (m, 2H), 1.88-1.70 (m, 4H), 1.44 (s, 6H).

### Example 15

N-(1-methoxy-1-methylethoxy)-N-methyl-5-[4-(4-chlorophenyl)benzoyl]pentanamide

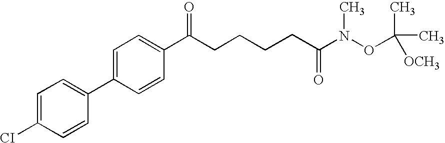

To a solution of the compound prepared in Example 14 (300 mg) in N,N-dimethylformamide (20 ml), sodium hydride (35 mg) was added at 0° C. and the mixture was stirred for 30 minutes at room temperature. The mixture was cooled to 0° C. then methyl iodide (54 μl) was added thereto and the mixture was stirred 1 hour at room temperature. The reaction mixture was extracted with ethyl acetate. The organic layer was washed with water and brine sequentially, dried over anhydrous magnesium sulfate, concentrated under reduced pressure. The residue was purified by column chromatography on silica gel (n-hexane:ethyl acetate=2:1, then n-hexane:ethyl acetate:triethylamine=1:1:0.02) to give the title compound (124 mg) having the following physical data.
TLC: Rf 0.52 (n-hexane:ethyl acetate=1:2);
NMR (CDCl₃): δ 8.02 (d, J=8.7 Hz, 2H), 7.64 (d, J=8.7 Hz, 2H), 7.55 (d, J=8.7 Hz, 2H), 7.43 (d, J=8.7 Hz, 2H), 3.35 (s, 3H), 3.29 (s, 3H), 3.03 (t, J=7.0 Hz, 2H), 2.44 (t, J=7.0 Hz, 2H), 1.85-1.70 (m, 4H), 1.43 (s, 6H).

### Example 16

N-(1-methoxy-1-methylethoxy)-N-methyl-6-[4-(4-chlorophenyl)phenyl]-6-hydroxyhexanamide

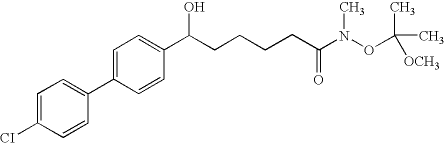

By the same procedure as described in Reference Example 3 using the compound prepared in Example 15 instead of the compound prepared in Reference Example 2, the compound of the present invention having the following physical data was obtained.
TLC: Rf 0.32 (n-hexane:ethyl acetate=1:2);
NMR (CDCl₃): δ 7.53 (d, J=8.5 Hz, 2H), 7.51 (d, J=8.5 Hz, 2H), 7.41 (d, J=8.5 Hz, 2H), 7.39 (d, J=8.5 Hz, 2H), 4.74 (dd, J=7.7, 5.5 Hz, 1H), 3.34 (s, 3H), 3.28 (s, 3H), 2.36 (t, J=7.5 Hz, 2H), 1.86-1.62 (m, 4H), 1.54-1.34 (m, 2H), 1.41 (s, 6H).

### Example 17

N-hydroxy-N-methyl-6-[4-(4-chlorophenyl)phenyl]-6-hydroxyhexanamide

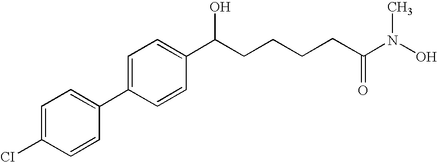

By the same procedure as described in Example 2 using the compound prepared in Example 16 instead of the compound prepared in Example 1, the compound of the present invention having the following physical data was obtained
TLC: Rf 0.34 (ethyl acetate);
NMR (DMSO-d₆): δ 9.71 (brs, 1H), 7.68 (d, J=8.5 Hz, 2H), 7.60 (d, J=8.5 Hz, 2H), 7.49 (d, J=8.5 Hz, 2H), 7.39 (d, J=8.5 Hz, 2H), 5.15 (d, J=5.0 Hz, 1H), 4.54 (q, J=5.0 Hz, 1H), 3.06 (s, 3H), 2.30 (t, J=7.0 Hz, 2H), 1.67-1.55 (m, 2H), 1.55-1.45 (m, 2H), 1.30-1.23 (m, 2H).

### Reference Example 16

6-[4-(4-chlorophenyl)phenyl]-6-hydroxyhexanoic acid

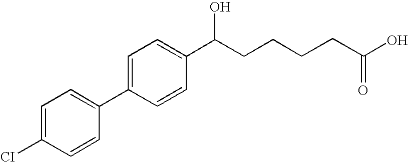

By the same procedure as described in Reference Example 3 using the compound prepared in Reference Example 15 instead of the compound prepared in Reference Example 2, the title compound having the following physical data was obtained.
TLC: Rf 0.52 (chloroform:tetrahydrofuran:acetic acid=10:4:1);
NMR (DMSO-d₆): δ 7.68 (d, J=8 Hz, 2H), 7.61 (d, J=8 Hz, 2H), 7.49 (d, J=8 Hz, 2H), 7.40 (d, J=8 Hz, 2H), 5.16 (bs, 1H), 4.65-4.40 (m, 1H), 2.18 (t, J=7 Hz, 2H), 1.80-1.05 (m, 6H).

### Example 18

N-methoxy-N-methyl-6-[4-(4-chlorophenyl)phenyl]-6-hydroxyhexanamide

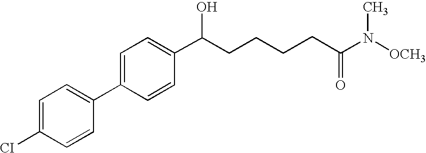

To a solution of the compound prepared in Reference Example 16 (318 mg) in N,N-dimethylformamide (20 ml), 1-hydroxybenzotriazole (229 mg) was added, cooled to 0° C., N-methoxy-N-methylamine hydrochloride (146 mg), triethylamine (553 μl) and 1-ethyl-3-[3-(dimethylamino)propyl]carbodiimide hydrochloride (287 mg) was sequentially added thereto and the mixture was stirred over night at room temperature. The reaction mixture was extracted with ethyl acetate. The organic layer was washed with saturated aqueous solution of sodium hydrogen carbonate, water and brine sequentially, dried over anhydrous magnesium sulfate, concentrated under reduced pressure. The residue was purified by column chromatography on silia gel (n-hexane:ethyl acetate=1:1) to give the compound in the present invention (207 mg) having the following physical data.
TLC: Rf 0.48 (ethyl acetate);
NMR (CDCl₃): δ 7.53 (d, J=8.4 Hz, 2H), 7.51 (d, J=8.4 Hz, 2H), 7.41 (d, J=8.4 Hz, 2H), 7.39 (d, J=8.4 Hz, 2H), 4.74 (dd, J=7.5, 5.5 Hz, 1H), 3.66 (s, 3H), 3.16 (s, 3H), 2.42 (t, J=7.5 Hz, 2H), 2.16 (m, 1H), 1.92-1.36 (m, 6H).

### Formulation Example 1

The following components were admixed in conventional method and punched out to give 100 tablets each containing 50 mg of active ingredient.

| | |
|---|---|
| N-Hydroxy-6-[(4-phenylbenzoyl)amino]hexanamide | 5.0 g |
| Calcium carboxymethylcellulose (disintegrant) | 0.2 g |
| Magnesium stearate (lubricant) | 0.1 g |
| Microcrystalline cellulose | 4.7 g |

### Formulation Example 2

The following components were admixed in conventional method and the solution was sterilized in conventional method, filled in ampoules 5 ml each and freeze-dried in conventional method to give 100 ampoules each containing 20 mg of active ingredient.

| | |
|---|---|
| N-Hydroxy-6-[(4-phenylbenzoyl)amino]hexanamide | 2.0 g |
| Mannitol | 20 g |
| Distilled water | 500 ml |

1. An IL-6, interleukin-6, production inhibitor, comprising a hydroxamic acid derivative of formula (I) 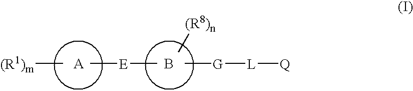
wherein, R¹ is
(a) C1-8 alkyl,
(b) C2-8 alkenyl,
(c) C2-8 alkynyl,
(d) halogen,
(e) nitro,
(f) cyano,
(g) trifluoromethyl,
(h) trifluoromethoxy,
(i) —OR²,
(j) —SR²,
(k) —NR³R⁴,
(l) —COR⁵,
(m) keto,
(n) Cycl,
(o) C1-8 alkyl substituted with —OR², —SR², —NR³R⁴, —COR⁵ or Cycl,
(p) SO₂R¹⁰,
(q) —SOR¹⁰,
(r) —O—(C1-8 alkylene)—OR¹¹,
(s) C1-8 alkyl substituted with cyano, 13 SO₂R¹⁰, —SOR¹⁰ or —O—(C1-8 alkylene)—OR¹¹,
(t) —O—(C1-8 alkylene)—NR¹²R¹³,
(u) —S—(C1-8 alkylene)—NR¹²R¹³,
(v) C1-8 alkyl substituted with —O—(C1-8 alkylene)—NR¹²R¹³— or —S—(C1-8 alkylene)—NR¹²R¹³,
(w) C2-8 alkenyl substituted with —OR², —SR², —NR³R⁴, —COR⁵, Cycl, cyano, —SO₂R¹⁰, —SOR¹⁰, —C—(C1-8 alkylene)—OR¹¹, —O—(C1-8 alkylene)—NR¹² R¹³ or —S—(C1-8 alkylene)—NR¹²R¹³, or
(x) C2-8 alkynyl substituted with —OR², —SR², —NR³R⁴, —COR⁵, Cycl, cyano, —SO₂R¹⁰, —SOR¹⁰, —O—(C1-8 alkylene)—OR¹¹, —O—(C1-8 alkylene)—NR¹²R¹³ or —S—(C1-8 alkylene)—NR¹²R¹³,
wherein R² is hydrogen, C1-8 alkyl, C2-9 acyl or Cycl;
R³ and R⁴ are each independently hydrogen, C1-8 alkyl, C2-9 acyl or Cycl;
R⁵ is hydroxy, C1-C8 alkyl, C1-8 alkoxy, —NR⁶R⁷ or Cycl;
R⁶ and R⁷ are each independently hydrogen, C1-8 alkyl or Cycl;
R¹⁰ is C1-8 alkyl or Cycl;
Cycl is
(a) C3-7 mono-carboxylic ring or
(b) 5 to 7 membered mono-heterocyclic ring containing 1 to 4 nitrogen atom(s), one oxygen atom and/or one sulfur atom;
R¹¹ is hydrogen, C1-8 alkyl, C2-9 acyl or Cycl;
R¹² and R¹³ are each independently hydrogen, C1-8 alkyl, C2-9 acyl or Cycl;
m is 0 or an integer of 1 to 5;
A is
(a) bond,
(b) C3-15 mono-, bi- or tri-carbocyclic ring or
(c) 5 to 18 membered mono-, bi- or tri-heterocyclic ring containing 1 to 4 nitrogen atom(s), 1 to 2 oxygen atom(s) and/or 1 to 2 sulfur atom(s);
E is
(a) bond,
(b) C1-8 alkylene,
(c) C2-8 alkenylene,
(d) C2-8 alkynylene,
(e) —O—,
(f) —SO₂NH—,
(g) —NHSO₂—,
(h) —CONH— or
(k) —NHCO—,
when E is (b) to (d), one saturated carbon atom in the alkylene, alkenylene or alkynylene is optionally displaced by one oxygen atom;
B is
(a) bond,
(b) C5-15 mono-, bi- or tri-carbocyclic ring or
(c) 5 to 18 membered mono-, bi- or tri-heterocyclic ring containing 1 to 4 nitrogen atom(s), 1 to 2 oxygen atom(s) and/or 1 to 2 sulfur atom(s);
R⁸ is
(a) C1-8 alkyl,
(b) C1-8 alkoxy,
(c) halogen,
(d) nitro,
(e) cyano,
(f) trifluoromethyl,
(g) trifluoromethoxy,
(h) hydroxy or
(i) C1-8 alkyl substituted with hydroxy,
when E is bond, R¹ and R⁸ are taken together to be C1-4 alkylene optionally;
n is 0 or an integer of 1 to 5;
G is
(a) bond,
(b) —NR²⁰CO—, wherein R²⁰ is hydrogen or C1-4 alkyl,
(c) —CONR²⁰—, wherein R²⁰ has the same meaning as defined hereinbefore,
(d) —O—,
(e) —S—,
(f) —SO—,
(g) —SO₂—,
(h) —SO₂NR²⁰—, wherein R²⁰ has the same meaning as defined hereinbefore,
(i) —CO—,
(j) —(C1-4 alkylene)—NR²³—, wherein R²³ is hydrogen, C1-8 alkyl or C1-4 alkoxycarbonyl,
(k) —(C1-4 alkylene)—OC(O)NH—, 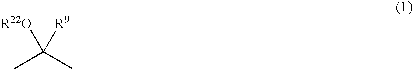
wherein R⁹ is hydrogen, hydroxy, C1-8 alkyl, C2-8 alkenyl, C2-8 alkynyl or C1-8 alkoxy, wherein the C1-8 alkyl, C2-8 alkenyl, C2-8 alkynyl or C1-8 alkoxy is optionally substituted with Cycl or C1-8 alkoxy; and R²² is hydrogen, C1-8 alkyl, C2-8 alkenyl, C2-8 alkynyl, C1-8 alkyl substituted with C1-8 alkoxy, C2-8 alkenyl substituted with C1-8 alkoxy, C2-8 alkynyl substituted with C1-8 alkoxy or C2 8 alkoxyalkyl substituted with Cycl, 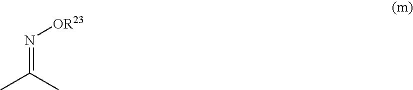
wherein R²³ is hydrogen, C1-8 alkyl, C2-8 alkenyl, C2-8 alkynyl or C2-8 alkoxyalkyl, 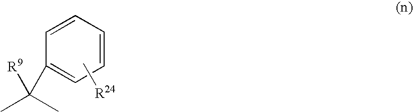
wherein R²⁴ has the same meaning as R¹, R⁹ has the same meaning as defined hereinbefore, 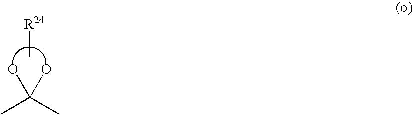
wherein 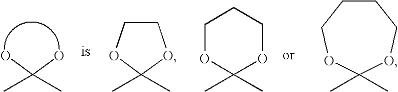
R²⁴ has the same meaning as defined hereinbefore, or
(p) 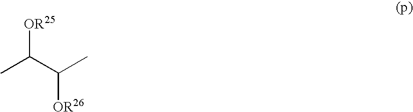
wherein R²⁵ and R²⁶ are each independently hydrogen, C1-8 alkyl, C2-8 alkenyl, C2-8 alkynyl or C2-8 alkoxyalkyl;
L is
(a) C1 8 alkylene,
(b) C2-8 alkenylene,
(c) C2-8 alkynylene,
(d) -(C2-8 alkenylene)-(C2-8 alkynylene)- or
(e) -(C2-8 alkynylene)-(C2-8 alkenylene)-
when L is (a) to (e), 1 to 2 saturated carbon atom(s) in the alkylene, alkenylene or alkynylene is optionally displaced by 1 or 2 —CONH—, —NHCO—, —CO—, —S—, —SO—, —SO₂—, —O—, —SO₂NH—, —NHSO₂—, phenylene, C3-8 cycloalkylene or thienylene, wherein the alkylene, alkenylene or alkynylene is optionally substituted with following substituents:
(a) C1-8 alkoxy;
(b) hydroxy,
(c) C1-4 alkoxy substituted with C1-4 alkoxy,
(d) Cycl, or
(e) Cycl substituted with C1-4 alkyl or C1-4 alkoxy;
Q is Q¹ or Q²,
Q¹ is 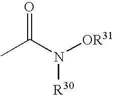
wherein R³⁰ is hydrogen or C1-8 alkyl, R³¹ is hydrogen, C1-8 alkyl or C2-8 alkoxyalkyl,
Q² is 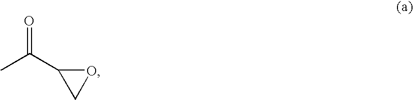
(b) —SR³²
wherein R³² is hydrogen, C1-8 alkyl or —C(O)—C1-8 alkyl, 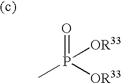
wherein R³³ is hydrogen or C1-4 alkyl, or
(d) —C(O)NR³⁴R³⁵
wherein R³⁴ is hydrogen or C1-8 alkyl, R³⁵ is hydrogen, C1-8 alkyl or NR³⁶R³⁷, wherein R³⁶ and R³⁷ are each independently hydrogen or C1-8 alkyl; and
wherein
(1) when G is 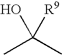
wherein R⁹ has the same meaning as defined hereinbefore, L is non-substituted tetramethylene and E is bond, —CH═CH— or —CH≡CH—, then Q is not Q¹, and
(2) A, E and B are not bond at same time,
an equivalent thereof, a non-toxic salt thereof or a prodrug thereof, as an active ingredient.

2. A pharmaceutical composition for the prevention and/or treatment of various inflammatory diseases, sepsis, multiple myeloma, plasma cell leukemia, osteoporosis, cachexia, psoriasis, nephritis, renal cell carcinoma, Kaposi's sarcoma, rheumatoid arthritis, hypergammaglobulinemia, Castleman's disease, intra-atrial myxoma, diabetes, autoimmune disease, hepatitis, colitis, graft-versus-host disease, infectious diseases, endometriosis and solid cancer, which comprises the L-6 production inhibitor according to claim 1, an equivalent thereof, a non-toxic salt thereof or a prodrug thereof, as an active ingredient.

3. The IL-6 production inhibitor according to claim 1, wherein the hydroxamic acid derivative of formula (I) is a hydroxamic acid derivative of formula (I-1) 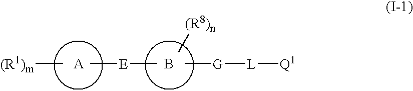
wherein the all symbols have the same meaning as defined in claim 1, a non-toxic salt thereof or a prodrug thereof, as an active ingredient.

4. The IL-6 production inhibitor according to claim 1, wherein the hydroxamic acid derivative of formula (I) is a equivalent of hydroxamic acid derivative of formula (I-2) 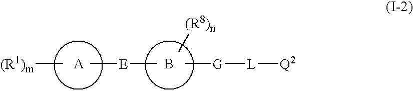
wherein the all symbols have the same meaning as defined in claim 1, to or a non-toxic salt thereof, as an active ingredient.

5. A hydroxamic acid derivative of the formula (I-1) 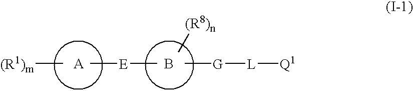
wherein the all symbols have the same meaning as defined in claim 1,
a non-toxic salt thereof or a prodrug thereof.

6. The hydroxamic acid derivative according to claim 5, wherein G is (b) —NR²⁰CO— or (c) —CONR²⁰—,
a non-toxic salt thereof or a prodrug thereof.

7. The hydroxamic acid derivative according to claim 5, wherein G 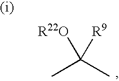
a non-toxic salt thereof or a prodrug thereof.

8. The hydroxamic acid derivative according to claim 5, wherein G is (i) —CO—,
a non-toxic salt thereof or a prodrug thereof.

9. The hydroxamic acid derivative according to claim 5, wherein G is (a) bond, (d) —O—, (e) —S—, (f) —SO—, (g) —SO₂—, (h) —SO₂NR²⁰—, (j) —(C1-4alkylene)—NR²³—, (k) —(C1-4 alkylene)—OC(O)NH—, 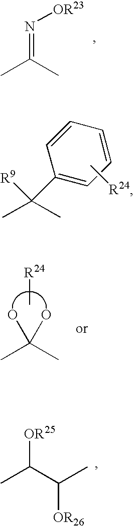
a non-toxic salt thereof or a prodrug thereof.

10. An equivalent of a hydroxamic acid derivative of the formula (I-2) 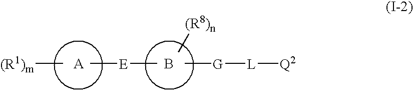
wherein the all symbols have the same meaning as defined in claim 1, or a non-toxic salt thereof.

11. The equivalent of the hydroxamic acid derivative according to claim 10, wherein Q² is 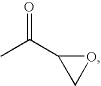
or a non-toxic salt thereof.

12. The equivalent of the hydroxamic acid derivative according to claim 10, wherein Q² is —SR³²,
or a non-toxic salt thereof.

13. The equivalent of the hydroxamic acid derivative according to claim 10, wherein Q² is 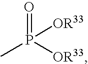
or a non-toxic salt thereof.

14. The equivalent of the hydroxamic acid derivative according to claim 10, wherein Q² is —C(O)NR³⁴R³⁵,
or a non-toxic salt thereof.

15. The hydroxamic acid derivative thereof according to claim 6, which is selected from the group consisting of
1) N-(1-methyl-1-methoxyethoxy)-6-[(4-phenylbenzoyl)amino]hexanamide,
2) N-hydroxy-6-[(4-phenylbenzoyl)amino]hexanamide,
3) N-hydroxy-3-[(4-phenylbenzoyl)amino]propanamide,
4) N-hydroxy-4-[(4-phenylbenzoyl)amino]butanamide,
5) N-hydroxy-5-[(4-phenylbenzoyl)amino]pentanamide,
6) N-hydroxy-7-[(4-phenylbenzoyl)amino]heptanamide,
7) N-hydroxy-3-{[4-(4-chlorophenyl)benzoyl]amino}propanamide,
8) N-hydroxy-5-{[4-(4-chlorophenyl)benzoyl]amino}pentanamide,
9) N-hydroxy-6-{[4-(4-chlorophenyl)benzoyl]amino}hexanamide,
10) N-hydroxy-7-{[4-(4-chlorophenyl)benzoyl]amino}heptanamide,
11) N-hydroxy-3-[(4-cyclohexylbenzoyl)amino]propanamide,
12) N-hydroxy-5-[(4-cyclohexylbenzoyl)amino]pentanamide,
13) N-hydroxy-6-[(4-cyclohexylbenzoyl)amino]hexanamide,
14) N-hydroxy-7-[(4-cyclohexylbenzoyl)amino]heptanamide,
15) N-hydroxy-3-(benzoylamino)propanamide,
16) N-hydroxy-4-(benzoylamino)butanamide,
17) N-hydroxy-5-(benzoylamino)pentanamide,
18) N-hydroxy-6-(benzoylamino)hexanamide,
19) N-hydroxy-7-(benzoylamino)heptanamide,
20) N-hydroxy-6-{[4-(4-cyanophenyl)benzoyl]amino}hexanamide,
21) N-hydroxy-6-{[4-(4-propylphenyl)benzoyl]amino}hexanamide,
22) N-hydroxy-6-[(4-phenoxybenzoyl)amino]hexanamide,
23) N-hydroxy-6-{[4-methoxyphenoxy)benzoyl]amino}hexanamide,
24) N-hydroxy-6-{[4-(3-phenoxyprop-1-ynyl)benzoyl]amino}hexanamide,
25) N-hydroxy-6-{[4-(3-methoxyprop-1-ynyl)benzoyl]amino}hexanamide,
26) N-hydroxy-6-[methyl(4-phenylbenzoyl)amino]hexanamide,
27) N-hydroxy-6-{[5-(4-methoxyphenyl)thiophen-2-ylcarbonyl]amino}hexanamide,
28) N-hydroxy-6-{[4-(3-methoxyphenoxy)benzoyl]amino}hexanamide,
29) (6S) N-hydroxy-7-othoxymethoxy-6-[(4-phenylbenzoyl)amino]heptanamide,
30) (6S)-N-hydroxy-7-hydroxy-6-[(4-phenylbenzoyl)amino]heptanamide,
31) (6S,2E)-N-hydroxy-7-ethoxymethoxy-2-methyl-6-[(4-phenylbenzoyl)amino]hept-2-enamide,
32) (6S)-N-hydroxy-7-ethoxymethoxy-2-methyl-6-[(4-phenylbenzoyl)amino]heptanamide,
33) N-hydroxy-5-[methyl(4-phenylbenzoyl)amino]pentanamide,
34) N-hydroxy-4-[(4-phenylbenzoyl)aminomethyl]benzamide,
35) N-hydroxy-3-[(1R,3R)-3-[(4-phenylbenzoyl)amino]cyclohexyl]propanamide,
36) (2E)-N-hydroxy-6-[(4-phenylbenzoyl)amino]hex-2-enamide,
37) (6R)-N-hydroxy-7-ethoxymethoxy-6-[(4-phenylbenzoyl)amino]heptanamide,
38) N-hydroxy-3-({2-[(4-phenylbenzoyl)amino]acetyl}amino)propanamide,
39) N-hydroxy-2-({3-[(4-phenylbenzoyl)amino]propanoyl}amino)acetamide,
40) N-hydroxy-5-[(4-phenylbenzoyl)aminomethyl]thiophene-2-carboxamide,
41) N-hydroxy-6-[(4-hydroxymethylbenzoyl)amino]hexanamide,
42) N-hydroxy-6-[(4-phenylcyclohexylcarbonyl)amino]hexanamide,
43) N-hydroxy-6-{[4-(4-methoxyphenyl)benzoyl]amino}hexanamide,
44) N-hydroxy-6-[(4-phenyl-3-cyclohexenylcarbonyl)amino]hexanamide,
45) N-hydroxy-6-[(6-dimethylaminopyridin-3-yl)amino]hexanamide,
46) N-hydroxy-6-{[4-(4-chlorophenyl)-3-hydroxymethylbenzoyl]amino}hexanamide,
47) N-hydroxy-6[(5-phenylpyrimidin-2-ylcarbonyl)amino]hexanamide,
48) N-hydroxy-6-[(4-cyclohexylphenyl)carbamoyl]hexanamide,
49) N-hydroxy-6-[(3-phenylphenyl)carbamoyl]hexanamide,
50) N-hydroxy-6-[(2-hydroxy-5-phenylphenyl)carbamoyl]hexanamide and
51) N-hydroxy-6-{[4(benzo[b]furan-2-yl)benzoyl]amino}hexanamide,
the non-toxic salt thereof or the prodrug thereof.

16. The hydroxamic acid derivative according to claim 7, which is selected from the group consisting of
1) N-hydroxy-6-methoxy-6-(4-phenoxyphenyl)hexanamide,
2) N-hydroxy-6-methoxy-6-[4-(morpholin-4-yl)phenyl]hexanamide,
3) N-hydroxy-6-methoxymethoxy-6-[4-(4-chlorophenyl)phenyl]-2-methylhexanamide,
4) (3E)-N-hydroxy-6-methoxymethoxy-6-[4-(4-chlorophenyl)phenyl]hex-3-enamide,
5) N-hydroxy-6-benzyloxymethoxy-6-[4-(4-chlorophenyl)phenyl]hexanamide,
6) (6R)-N-hydroxy-6-[4-(4-ethylphenyl)phenyl]-6-methoxyhexanamide,
7) N-hydroxy-6-[4-(4-chlorophenyl)phenyl]-6-methoxymethoxyhexanamide,
8) N-hydroxy-6-[4-(4-chlorophenyl)phenyl]-6-methoxyhexanamide,
9) N-hydroxy-4-[4-(4-chlorophenyl)phenyl]-4-methoxymethoxybutanamide,
10) N-hydroxy-5-[4-(4-chlorophenyl)phenyl]-5-methoxymethoxypentanamide,
11) N-hydroxy-6-hydroxy-6-(4-methylphenyl)hexanamide,
12) N-hydroxy-6-hydroxy-6-phenylhexanamide,
13) N-hydroxy-6-[4-(4-chlorophenyl)phenyl]-2,2-dimethyl-6-hydroxyhexanamide,
14) N-hydroxy-4-[4-(4-chlorophenyl)phenyl]-4-hydroxybutanamide,
15) N-hydroxy-2-{3-hydroxy-3-[4-(4-chlorophenyl)phenyl]propylthio}acetamide,
16) N-hydroxy-2-{3-hydroxy-3-[4-(4-chlorophenyl)phenyl]propylthio}propanamide,
17) N-hydroxy-7-hydroxy-7-[4-(4-chlorophenyl)phenyl]heptanamide,
18) N-hydroxy-8-[4-(4-chlorophenyl)phenyl]-8-hydroxy]octanamide,
19) N-hydroxy-2-{3-[4-(4-chlorophenyl)phenyl]-3-hydroxypropylthio}-2-methylpropanamide,
20) N-hydroxy-5-[4-(4-chlorophenyl)phenyl]-5-hydroxypentanamide,
21) N-hydroxy-6-hydroxy-6-(4-iodophenyl)hexanamide,
22) N-hydroxy-6-hydroxy-6-(naphthalen-2-yl)hexanamide,
23) (7E)-N-hydroxy-6-hydroxy-8-phenyloct-7-enamide,
24) (7E,9E)-N-hydroxy-6-hydroxy-10-phenyldec-7,9-dienamide,
25) N-hydroxy-6-(benzo[b]thiophen-2-yl)-6-hydroxyhexanamide,
26) N-hydroxy-6-(benzo[b]thiophen-3-yl)-6-hydroxyhexanamide,
27) N-hydroxy-6-hydroxy-6-(4-phenoxyphenyl)hexanamide,
28) N-hydroxy-6-[4-(4-chlorophenyl)phenyl]-6-hydroxyhex-2-ynamide,
29) N-hydroxy-6-hydroxy-6-{4-[2-(pyridin-4-yl)ethyl]phenyl}hexanamide,
30) N-hydroxy-6-hydroxy-6-(4-phenethylphenyl)hexanamide,
31) N-hydroxy-6-[4-(4-chlorophenyl)phenyl]-6-hydroxy-2-methylhexanamide,
32) N-hydroxy-6-[4-(4-chlorophenyl)phenyl]-6-hydroxy-2-(pyridin-4-ylmethyl)hexanamide,
33) N-hydroxy-8-(1,3-dioxan-2-yl)-6-hydroxy-6-(4-phenylphenyl)octanamide,
34) N-hydroxy-4-[1-hydroxy-1-(4-phenylphenyl)methyl]benzamide,
35) N-hydroxy-3-{2-[4-(4-chlorophenyl)phenyl]-2-hydroxyethylthio}propanamide,
36) N-hydroxy-3-{2-[4-(4-chlorophenyl)phenyl]-2-hydroxyethoxy}benzamide,
37) (2E)-N-hydroxy-6-[4-(4-chlorophenyl)phenyl]-6-hydroxyhex-2-enamide,
38) N-hydroxy-2-{2-[4-(4-chlorophenyl)phenyl]-2-hydroxyethylthio}acetamide,
39) N-hydroxy-4-{2-[4-(4-chlorophenyl)phenyl]-2-hydroxyethoxy}benzamide,
40) (2E)-N-hydroxy-6-[4-(4-chlorophenyl)phenyl]-6-hydroxy-2-methylhex-2-enamide,
41) N-hydroxy-6-[4-(4-chlorophenyl)phenyl]-6-hydroxy-9-methoxynon-7-ynamide,
42) (6R)-N-hydroxy-6-hydroxy-6-[4-(3-phenoxyprop-1ynyl)phenyl]hexanamide,
43) (6R)-N-hydroxy-6-[4-(benzoylamino)phenyl]-6-hydroxyhexanamide,
44) N-hydroxy-6-hydroxy-6-[4-(4-hydroxybut-1-ynyl)phenyl]hexanamide,
45) (6R)-N-hydroxy-6-hydroxy-6-[4-(phenylcarbamoyl)phenyl]hexanamide,
46) (7E)-N-hydroxy-6-methoxy-8-phenyloct-7-enamide,
47) N-(1-methoxy-1-methylethoxy)-N-methyl-6-[4-(4-chlorophenyl)phenyl]-6-hydroxyhexanamide,
48) N-hydroxy-N-methyl-6-[4-(4-chlorophenyl)phenyl]-6-hydroxyhexanamide and
49) N-methoxy-N-methyl-6-[4-(4-chlorophenyl)phenyl]-6-hydroxyhexanamide,
a non-toxic salt thereof or a prodrug thereof.

17. The hydroxamic acid derivative according to claim 8, which is selected from the group consisting of
1) N-hydroxy-6-[4-(4-chlorophenyl)benzoyl]hexanamide,
2) N-hydroxy-4-[4-(4-chlorophenyl)benzoyl]butanamide,
3) N-hydroxy-7-[4-(4-chlorophenyl)benzoyl]heptanamide,
4) N-hydroxy-5-(naphthalen-2-ylcarbonyl)pentamide,
5) (7E)-N-hydroxy-6-oxo-8-phenyloct-7-enamide,
6) (7E,9E)-N-hydroxy-6-oxo-10-phenyldec-7,9-dienamide,
7) N-hydroxy-5-(benzo[b]thiophen-2-ylcarbonyl)pentanamide,
8) N-hydroxy-5-(benzo[b]thiophen-3-ylcarbonyl)pentanamide,
9) N-hydroxy-5-(4-phenoxybenzoyl)pentanamide,
10) N-hydroxy-4-[4-(4-chlorophenyl)benzoylmethylthio]butanamide,
11) N-hydroxy-3-[4-(4-chlorophenyl)benzoylmethoxy]benzamide,
12) N-hydroxy-3-[4-(4-chlorophenyl)benzoylmethylthio]propanamide,
13) N-hydroxy-4-[4-(4-chlorophenyl)benzoylmethoxy]benzamide,
14) N-(1-methyl-1-methoxyethoxy)-5-[4-(4-chlorophenyl)benzoyl]pentanamide or
15) N-(1-methoxy-1-methylethoxy)-N-methyl-5-[4-(4-chlorophenyl)benzoyl]penanamide,
a non-toxic salt thereof or a prodrug thereof.

18. The hydroxamic acid derivative according to claim 9, which is selected from the group consisting of
1) N-hydroxy-3-[(4-phenylbenzylcarbonyl)amino]propanamide,
2) N-hydroxy-4-[(4-phenylbenzyl)carbamoyl]butanamide,
3) N-hydroxy-4-[4-(4-chlorophenyl)benzyloxy]butanamide,
4) N-hydroxy-5-[4-(4-chlorophenyl)-2-hydroxymethylphenoxy]pentanamide,
5) N-hydroxy-5-[4-(4-chlorophenyl)-2-hydroxyphenoxy]pentanamide,
6) N-hydroxy-5-[4-(4-cyanophenyl)phenoxy]pentanamide,
7) N-hydroxy-6-[4-(4-cyanophenyl)phenoxy]hexanamide,
8) N-hydroxy-5-[4-(4-chlorophenyl)phenoxy]pentanamide,
9) N-hydroxy-7-[4-(4-cyanophenyl)phenoxy]heptanamide,
10) N-hydroxy-6-[4-(4-chlorophenyl)phenoxy]hexanamide,
11) N-hydroxy-7-[4-(4-chlorophenyl)phenoxy]heptanamide,
12) N-hydroxy-5-[4-(4-chlorophenyl)phenylthio]pentanamide,
13) N-hydroxy-7-[4-(4-chlorophenyl)phenylthio]heptanamide,
14) N-hydroxy-4-[4-(4-chlorophenyl)benzylthio]butanamide,
15) N-hydroxy-4-{[4-(4-chlorophenyl)phenylsulfoyl]amino}butanamide,
16) N-hydroxy-6-{[4-(4-chlorophenyl)phenylsulfonyl]amino}hexanamide,
17) N-hydroxy-5-[4-(4-chlorophenyl)phenylsulfinyl]pentanamide,
18) N-hydroxy-7-[4-(4-chlorophenyl)phenylsulfinyl]heptanamide,
19) N-hydroxy-5-[4-(4-chlorophenyl)phenylsulfonyl]pentanamide,
20) N-hydroxy-7-[4-(4-chlorophenyl)phenylsulfonyl]heptanamide,
21) N-hydroxy-4-{1-[4-(4-chlorophenyl)phenyl]-2-(methoxymethoxy)ethoxy}butanamide,
22) N-hydroxy-6-(4-methoxyphenyl)-6-4-phenylphenyl)hexanamide,
23) N-hydroxy-5-{2-[4-(4-chlorophenyl)phenyl]-1,3-dioxolan-2-yl}pentanamide,
24) N-hydroxy-4-{2-[4-(4-chlorophenyl)phenyl]-1,3-dioxolan-2-ylmethoxy}benzamide,
25) N-hydroxy-5-{2-[4-(4-chlorophenyl)phenyl]-4-methoxymethyl-1,3-dioxolan-2-yl}pentanamide,
26) N-hydroxy-5-{2-[4-(4-chlorophenyl]-4-(4-hydroxybutyl)-1,3-dioxolan-2-yl}pentanamide,
27) (2E)-N-hydroxy-5-{3-[(phenylsulfonyl)amino]phenyl}pent-2-en-4-ynamide,
28) N-hydroxy-4-{1-[4-(4-chlorophenyl)phenyl]-2-hydroxyethoxy}butanamide,
29) N-hydroxy-6-[4-(4-chlorophenyl)phenyl]-7-hydroxyheptanamide,
30) N-hydroxy-6-[4-(4-chlorophenyl)phenyl]-6-hydroxy-6-(4-methoxyphenyl)hexanamide,
31) N-hydroxy-6-(4-phenylphenyl)-5,6-dihydroxyhexanamide,
32) N-hydroxy-6-[4-(4-chlorophenyl)phenyl]-6-hydroxyiminohexanamide,
33) N-hydroxy-6-[4-(4-chlorophenyl)phenyl]heptanamide,
34) N-hydroxy-6-[4-(4-chlorophenyl)phenyl]hexanamide,
35) N-hydroxy-3-[(4-phenylbenzyloxycarbonyl)amino]propanamide,
36) N-hydroxy-2-[(4-phenylbenzyloxycarbonyl)amino]acetamide,
37) N-hydroxy-4-[4-(benzo[b]furan-2-yl)benzyloxyl]butanamide,
38) N-hydroxy-4-{t-butoxycarbonyl[4-(4-chlorophenyl)phenylmethyl]amino}butanamide,
39) N-hydroxy-5-{t-butoxycarbonyl[4-(4-chlorophenyl)phenylmethyl]amino}pentanamide,
40) N-hydroxy-6-{t-butoxycarbonyl[4-(4-chlorophenyl)phenylmethyl]amino}hexanamide,
41) N-hydroxy-4-{[4-(4-chlorophenyl)phenylmethyl]amino}butanamide,
42) N-hydroxy-5-{[4-(4-chlorophenyl)phenylmethyl]amino}pentanamide and
43) N-hydroxy-6-{[4-(4-chlorophenyl)phenylmethyl]amino}hexanamide,
a non-toxic salt thereof or a prodrug thereof.

19. The equivalent of the hydroxamic acid derivative according to claim 10, which is selected from the group consisting of
1) 6-hydroxy-6-[4-(4-methylphenyl)phenyl]hexylphosphonic acid diethyl ester,
2) 6-hydroxy-6-[4-(4-methylphenyl)phenyl]hexylphosphonic acid
3) 5-acetylthio-1-[4-(4-chlorophenyl)phenyl]-1-methoxymethoxypentane,
4) 5-acetylthio-1-[4-(4-chlorophenyl)phenyl]pentanol,
5) 1-[4-(4-chlorophenyl)phenyl]-5-mercaptoentanol,
6) N-amino-6-[4-(4-chlorophenyl)phenyl]-6-hydroxyhexanamide,
7) 6-[4-(4-chlorophenyl)phenyl]-6-hydroxyhexanamide,
8) N-methoxy-N-methyl-6-[4-(4-methylphenyl)phenyl]-6-hydroxyhexanamide and
9) 6-hydroxy-6-[4-(4-methylphenyl)phenyl]-1-(oxiran-2-yl)hexan-1-one, or
a non-toxic salt thereof.

20. A method for inhibiting production of an IL-6, interleukin-6which comprises administering the IL-6 production inhibitor according to claim 1, an equivalent thereof, a non toxic salt thereof or a prodrug thereof.

21. A method for the prevention and/or treatment of various inflammatory diseases, sepsis, multiple myeloma, plasma cell leukemia, osteoporosis, cachexia, psoriasis, nephritis, renal cell carcinoma, Kaposi's sarcoma, rheumatoid arthritis, hypergammaglobulinemia, Castleman's disease, intra-atrial myxoma, diabetes, autoimmune disease, hepatitis, colitis, graft-versus-host disease, infectious diseases, endometriosis and solid cancer, which comprises administering the IL-6 production inhibitor according to claim 1, an equivalent thereof, a non-toxic salt thereof or a prodrug thereof.

22. The method according to claim 21, wherein hypergammaglobulinemia is gammophathy.

23. The method according to claim 21, wherein solid cancer is the one selected from the group consisting of brain tumor, head and neck tumor, thyroid cancer, esophageal cancer, gastric cancer, colorectal cancer, liver cancer, gallbladder cancer, bileduct cancer, pancreatic cancer, lung cancer, breast cancer, cervical cancer, endometrial carcinoma, ovarian cancer, carcinoma of the prostate, orchioncus, bladder carcinoma, renal pelvic tumor, ureteral tumor, adrenal tumor, neuroma, glioma, osteoncus, rhabdomyosarcoma, osteosarcoma, soft tissue tumors, oxyphilic granuloma, malignant melanoma, cutaneous carcinoma, glioblastoma or Wilms tumor.

24. The method according to claim 23, wherein colorectal cancer is colon cancer or rectal cancer.

25. A method for inhibiting IL-6 production, which comprises administering the IL-6 production inhibitor according to claim 3, a non-toxic salt thereof or a prodrug thereof.

26. A method for inhibiting IL-6 production, which comprises administering the IL-6 production inhibitor according to claim 4, or a non-toxic salt thereof.

27. A method for the prevention and/or treatment of various inflammatory diseases, sepsis, multiple myeloma, plasma cell leukemia, osteoporosis, cachexia, psoriasis, nephritis, renal cell carcinoma, Kaposi's sarcoma, rheumatoid arthritis, hypergammaglobulinemia, Castleman's disease, intra-atrial myxoma, diabetes, autoimmune disease, hepatitis, colitis, graft-versus-host disease, infectious diseases, endometriosis and solid cancer, which comprises administering the hydroxamic acid derivative of formula (I-1) according to claim 5, a non-toxic salt thereof or a prodrug thereof.

28. The method according to claim 27, wherein hypergammaglobulinemia is gammophathy.

29. The method according to claim 27, wherein solid cancer is the one selected from the group consisting of brain tumor, head and neck tumor, thyroid cancer, esophageal cancer, gastric cancer, colorectal cancer, liver cancer, gallbladder cancer, bileduct cancer, pancreatic cancer, lung cancer, breast cancer, cervical cancer, endometrial carcinoma, ovarian cancer, carcinoma of the prostate, orchioncus, bladder carcinoma, renal pelvic tumor, ureteral tumor, adrenal tumor, neuroma, glioma, osteoncus, rhabdomyosarcoma, osteosarcoma, soft tissue tumors, oxyphilic granuloma, malignant melanoma, cutaneous carcinoma, glioblastoma or Wilms tumor.

30. The method according to claim 29, wherein colorectal cancer is colon cancer or rectal cancer.

31. A method for the prevention and/or treatment of various inflammatory diseases, sepsis, multiple myeloma, plasma cell leukemia, osteoporosis, cachexia, psoriasis, nephritis, renal cell carcinoma, Kaposi's sarcoma, rheumatoid arthritis, hypergammaglobulinemia, Castleman's disease, intra-atrial myxoma, diabetes, autoimmune disease, hepatitis, colitis, graft-versus-host disease, infectious diseases, endometriosis and solid cancer, which comprises administering the equivalent of the hydroxamic acid derivative of formula (I-2) according to claim 10, or a non-toxic salt thereof.

32. The method according to claim 31, wherein hypergammaglobulinemia is gammophathy.

33. The method according to claim 31, wherein solid cancer is the one selected from the group consisting of brain tumor, head and neck tumor, thyroid cancer, esophageal cancer, gastric cancer, colorectal cancer, liver cancer, gallbladder cancer, bileduct cancer, pancreatic cancer, lung cancer, breast cancer, cervical cancer, endometrial carcinoma, ovarian cancer, carcinoma of the prostate, orchioncus, bladder carcinoma, renal pelvic tumor, ureteral tumor, adrenal tumor, neuroma, glioma, osteoncus, rhabdomyosarcoma, osteosarcoma, soft tissue tumors, oxyphilic granuloma, malignant melanoma, cutaneous carcinoma, glioblastoma or Wilms tumor.

34. The method according to claim 33, wherein colorectal cancer is colon cancer or rectal cancer.

35. The pharmaceutical composition according to claim 2, wherein hypergammaglobulinemia is gammophathy.

36. The pharmaceutical composition according to claim 2, wherein solid cancer is the one selected from the group consisting of brain tumor, head and neck tumor, thyroid cancer, esophageal cancer, gastric cancer, colorectal cancer, liver cancer, gallbladder cancer, bileduct cancer, pancreatic cancer, lung cancer, breast cancer, cervical cancer, endometrial carcinoma, ovarian cancer, carcinoma of the prostate, orchioncus, bladder carcinoma, renal pelvic tumor, ureteral tumor, adrenal tumor, neuroma, glioma, osteoncus, rhabdomyosarcoma, osteosarcoma, soft tissue tumors, oxyphilic granuloma, malignant melanoma, cutaneous carcinoma, glioblastoma or Wilms tumor.

37. The pharmaceutical composition according to claim 36, wherein colorectal cancer is colon cancer or rectal cancer.
